(19) United States
(12) Patent Application Publication
(10) Pub. No.: US 2007/0185149 A1
(43) Pub. Date: Aug. 9, 2007

(54) Novel amido-substituted hydroxy-6-phenylphenanthridines and their use as pde4 inhibtors

(75) Inventor: Ulrich Kautz, Allensbach (DE)

Correspondence Address: NATH & ASSOCIATES PLLC, 112 South West Street, Alexandria, VA, 22314, US

(73) Assignee: ALTANA PHARMA AG, 78467 KONSTANZ (DE)

(21) Appl. No.: 10/591,480

(22) PCT Filed: Mar. 9, 2005
(86) PCT No.: PCT/EP05/51054

§ 371 Date: Sep. 27, 2006

(30) Foreign Application Priority Data
Mar. 10, 2004 (EP) 04100990.3
Dec. 17, 2004 (EP) 04106677.0

(51) Int. Cl. A61K 31/44; C07D 221/06

(52) U.S. Cl. 514/298; 546/101

(57) ABSTRACT

Compounds of the formula I 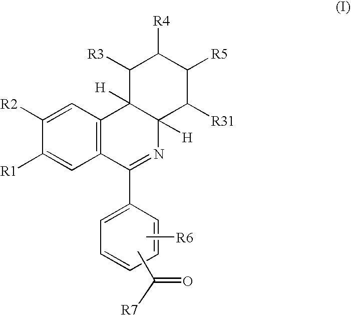 in which the substituents have the definitions provided in the specification, are novel, effective PDE4 inhibitors.

### FIELD OF APPLICATION OF THE INVENTION

The invention relates to novel amido-substituted hydroxy-6-phenylphenanthridine derivatives, which are used in the pharmaceutical industry for the production of pharmaceutical compositions.

### KNOWN TECHNICAL BACKGROUND

The International Patent applications WO99/57118 and WO02/05616 describe 6-phenylphenanthridines as PDE4 inhibitors.

In the International Patent application W099/05112 substituted 6-alkylphenanthridines are described as bronchial therapeutics.

In the European Patent application EP 0490823 dihydroisoquinoline derivatives are described which are useful in the treatment of asthma.

The international application WO 97128131 discloses phenanthridines as bronchial therapeutic agents,

The international application WO 99105113 discloses 6-phenylphenanthridines as bronchial therapeutics.

The international application WO 00/42020 discloses phenylphenanthridines with PDE4 inhibiting properties.

The international application WO 0205616 discloses phenylphenanthridines with PDE4 inhibiting properties

The International Patent applications WO2004/019944 and WO2004/019945 disclose hydroxy-substituted 6-phenylphenanthridines as PDE4 inhibitors.

### DESCRIPTION OF THE INVENTION

It has now been found that the novel amido-substituted 2- or 3-hydroxy-6-phenylphenanthridines described in greater detail below differ from the previously known compounds by unanticipated and sophisticated structural alterations and have surprising and particularly advantageous properties.

The invention thus relates to compounds of formula I, 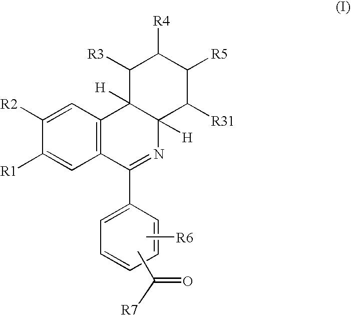
in which
R1 is hydroxyl, 1-4C-alkoxy, 3-7C-cycloalkoxy, 3-7C-cycloalkylmethoxy, 2,2-difluoroethoxy, or completely or predominantly fluorine-substituted 1-4C-alkoxy,
R2 is hydroxyl, 1-4C-alkoxy, 3-7C-cycloalkoxy, 3-7C-cycloalkylmethoxy, 2,2-difluoroethoxy, or completely or predominantly fluorine-substituted 1-4C-alkoxy,
   or in which
R1 and R2 together are a 1-2C-alkylenedioxy group,
R3 is hydrogen or 1-4C-alkyl,
R31 is hydrogen or 1-4C-alkyl,
   either, in a first embodiment (embodiment a) according to the present invention,
R4 is —O—R41, in which
R41 is hydrogen, 1-4C-alkyl, 1-4C-alkoxy-1-4C-alkyl, hydroxy-2-4C-alkyl, 1-7C-alkylcarbonyl, or completely or predominantly fluorine-substituted 1-4C-alkyl, and
R5 is hydrogen or 1-4C-alkyl,
   or, in a second embodiment (embodiment b) according to the present invention,
R4 is hydrogen or 1-4C-alkyl, and
R5 is —O—R51, in which
R51 is hydrogen, 1-4C-alkyl, 1-4C-alkoxy-1-4C-alkyl, hydroxy-2-4C-alkyl, 1-7C-alkylcarbonyl, or completely or predominantly fluorine-substituted 1-4C-alkyl,
R6 is hydrogen, halogen, 1-4C-alkyl or 1-4C-alkoxy,
   either,
   in a first aspect (aspect 1) according to the present invention,
R7 is —N(R8)R9, in which
R8 is hydrogen, 1-4C-alkyl or 1-4C-alkoxy-2-4C-alkyl,
R9 is hydrogen, 1-4C-alkyl, mono- or di-1-4C-alkoxy-2-4C-alkyl, hydroxy-2-4C-alkyl, mono- or di-1-4C-alkoxycarbonyl-1-4C-alkyl, Har1, pyridinyl-1-4C-alkyl, 3-7C-cycloalkyl, or 2-4C-alkyl substituted by —NR(93)R94, in which
Har1 is optionally substituted by R91 and/or R92, and is a 5 to 10-membered monocylic or fused bicyclic unsaturated heteroaryl radical comprising 1 to 4 heteroatoms selected independently from the group consisting of oxygen, nitrogen and sulfur, in which
R91 is 1-4C-alkyl or 1-4C-alkoxy,
R92 is 1-4C-alkyl or 1AC-alkoxy,
R93 is hydrogen or 1-4C-alkyl,
R94 is hydrogen or 1-4C-alkyl,
   or R93 and R94 together and with inclusion of the nitrogen atom, to which they are attached, form a heterocyclic ring Het1, in which
Het1 is optionally substituted by R931, and is a 3- to 7-membered saturated monocyclic heterocyclic ring radical comprising the nitrogen atom, to which R93 and R94 are bonded, and optionally one further heteroatom selected from the group consisting of oxygen, nitrogen and sulfur, in which
R931 is 1-4C-alkyl,
   or R8 and R9 together and with inclusion of the nitrogen atom, to which they are attached, form a heterocyclic ring Het2, in which
Het2 is optionally substituted by R10, and is a 3- to 7-membered saturated monocyclic heterocyclic ring radical comprising the nitrogen atom, to which R8 and R9 are bonded, and optionally one further heteroatom selected from the group consisting of oxygen, nitrogen and sulfur, in which
R10 is 1-4C-alkyl, —C(O)R11, pyridyl, 2-4C-alkyl substituted by —NR(14)R15, or 1-4C-alkyl substituted by —C(O)N(R16)R17, in which
R11 is 1-4C-alkyl substituted by —NR(12)R13, in which
R12 is hydrogen or 1-4C-alkyl,
R13 is hydrogen or 1-4C-alkyl,
   or R12 and R13 together and with inclusion of the nitrogen atom, to which they are attached, form a heterocyclic ring Het3, in which
Het3 is optionally substituted by R121, and is a 3- to 7-membered saturated monocyclic heterocyclic ring radical comprising the nitrogen atom, to which R12 and R13 are bonded, and optionally one further heteroatom selected from the group consisting of oxygen, nitrogen and sulfur, in which
R121 is 1-4C-alkyl,
R14 is hydrogen or 1-4C-alkyl,
R15 is hydrogen or 1-4C-alkyl,
   or R14 and R15 together and with inclusion of the nitrogen atom, to which they are attached, form a heterocyclic ring Het4, in which
Het4 is optionally substituted by R141, and is a 3- to 7-membered saturated monocyclic heterocyclic ring radical comprising the nitrogen atom, to which R14 and R15 are bonded, and optionally one further heteroatom selected from the group consisting of oxygen, nitrogen and sulfur, in which
R141 is 1-4C-alkyl,
R16 is hydrogen, 1-4C-alkyl or pyridyl,
R17 is hydrogen or 1-4C-alkyl,
   or R16 and R17 together and with inclusion of the nitrogen atom, to which they are attached, form a heterocyclic ring Het5, in which
Het5 is optionally substituted by R161, and is a 3- to 7-membered saturated monocyclic heterocyclic ring radical comprising the nitrogen atom, to which R16 and R17 are bonded, and optionally one further heteroatom selected from the group consisting of oxygen, nitrogen and sulfur, in which
R141 is 1-4C-alkyl,
   or,
   in a second aspect (aspect 2) according to the present invention,
R7 is —NH—N(R18)R19, in which
R18 is hydrogen,
R19 is —C(O)R20, or R21-substituted phenyl, in which
R20 is Har2, Het6, or Aryl-1-4C-alkyl, in which
Har2 is optionally substituted by R201 and/or R202, and is a 5- to 10-membered monocylic or fused bicyclic unsaturated heteroaryl radical comprising 1 to 4 heteroatoms selected independently from the group consisting of oxygen, nitrogen and sulfur, in which
R201 is 1-4C-alkyl or 1-C-alkoxy,
R202 is 1-4C-alkyl or 1-4C-alkoxy,
Het6 is optionally substituted by R203 and/or R204, and is a monocylic 3- to 7-membered saturated heterocyclic ring radical comprising one to three heteroatoms, each of which is selected from the group consisting of nitrogen, oxygen and sulfur, in which
R203 is 1-4C-alkyl,
R204 is 1-4C-alkyl,
Aryl is R205- and/or R206-substituted phenyl,
R205 is 1-4C-alkoxy
R206 is 1-4C-alkoxy
R21 is aminosulphonyl,
   or R18 and R19 together and with inclusion of the nitrogen atom, to which they are attached, form a heterocyclic ring Het7, in which
Het7 is optionally substituted by R181, and is a 3- to 7-membered saturated monocyclic heterocyclic ring radical comprising the nitrogen atom, to which R18 and R19 are bonded, and optionally one further heteroatom selected from the group consisting of oxygen, nitrogen and sulfur, in which
R181 is 1-4C-alkyl,
   and the salts, the N-oxides and the salts of the N-oxides of these compounds.

1-4C-Alkyl represents a straight-chain or branched alkyl radical having 1 to 4 carbon atoms. Examples which may be mentioned are the butyl, isobutyl, sec-butyl, tert-butyl, propyl, isopropyl and preferably the ethyl and methyl radicals.

2-4C-Alkyl represents a straight-chain or branched alkyl radical having 2 to 4 carbon atoms. Examples which may be mentioned are the butyl, isobutyl, sec-butyl, tert-butyl, propyl, isopropyl and preferably the ethyl radical.

1-7C-Alkyl represents a straight-chain or branched alkyl radical having 1 to 7 carbon atoms. Examples which may be mentioned are the heptyl, isoheptyl (5-methylhexyl), hexyl, isohexyl (4-methylpentyl), neohexyl (3,3-dimethylbutyl), pentyl, isopentyl (3-methylbutyl), neopentyl (2,2-dimethylpropyl), butyl, isobutyl, sec-butyl, tert-butyl, propyl, isopropyl, ethyl or methyl radicals.

3-7C-Cycloalkyl represents cyclopropyl, cyclobutyl, cyclopentyl, cyclohexyl and cycloheptyl, of which cyclopropyl, cyclobutyl and cyclopentyl are preferred.

1-4C-Alkoxy represents radicals which, in addition to the oxygen atom, contain a straight-chain or branched alkyl radical having 1 to 4 carbon atoms. Examples which may be mentioned are the butoxy, isobutoxy, sec-butoxy, tert-butoxy, propoxy, isopropoxy and preferably the ethoxy and methoxy radicals.

3-7C-Cycloalkoxy represents cyclopropyloxy, cyclobutyloxy, cyclopentyloxy, cyclohexyloxy and cycloheptyloxy, of which cyclopropyloxy, cyclobutyloxy and cyclopentyloxy are preferred.

3-7C-Cycloalkyl methoxy represents cyclopropyl methoxy, cyclobutylmethoxy, cyclopentylmethoxy, cyclohexylmethoxy and cycloheptylmethoxy, of which cyclopropylmethoxy, cyclobutylmethoxy and cyclopentylmethoxy are preferred.

As completely or predominantly fluorine-substituted 1-4C-alkoxy, for example, the 2,2,3,3,3-pentafluoropropoxy, the perfluoroethoxy, the 1,2,2-trifluoroethoxy, in particular the 1,1,2,2-tetrafluoroethoxy, the 2,2,2-trifluoroethoxy, the trifluoromethoxy and preferably the difluoromethoxy radicals may be mentioned.

“Predominantly” in this connection means that more than half of the hydrogen atoms of the 1-4C-alkoxy radicals are replaced by fluorine atoms.

As completely or predominantly fluorine-substituted 1-4C-alkyl, for example, the 2,2,3,3,3-pentafluoropropyl, the perfluoroethyl, the 1,2,2-trifluoroethyl, in particular the 1,1,2,2-tetrafluoroethyl, the 2,2,2-trifluoroethyl, the trifluoromethyl and particularly the difluoromethyl radicals may be mentioned. “Predominantly” in this connection means that more than half of the hydrogen atoms of the 1-4C-alkyl radicals are replaced by fluorine atoms.

1-2C-Alkylenedioxy represents, for example, the methylenedioxy [—O—CH—O₂—] and the ethylenedioxy [—O—CH₂CH₂—O—] radicals.

1-4C-Alkoxy-1-4C-alkyl represents one of the abovementioned 1-4C-alkyl radicals, which is substituted by one of the abovementioned 1-4C-alkoxy radicals. Examples which may be mentioned are the methoxymethyl, the methoxyethyl and the isopropoxyethyl radicals, particularly the 2-methoxyethyl and the 2-isopropoxyethyl radicals.

1-4C-Alkoxy-2-4C-alkyl represents one of the abovementioned 2-4C-alkyl radicals, which is substituted by one of the abovemenfioned 1-4C-alkoxy radicals. Examples which may be mentioned are the methoxyethyl and the isopropoxyethyl radicals, particularly the 2-methoxyethyl and the 2-isopropoxyethyl radicals.

1-7C-Alkylcarbonyl represents a radical which, in addition to the carbonyl group, contains one of the abovementioned 1-7C-alkyl radicals. Examples which may be mentioned are the acetyl, propionyl, butanoyl and hexanoyl radicals.

Hydroxy-2-4C-alkyl represents 2-4C-alkyl radicals, which are substituted by a hydroxyl group. Examples which may be mentioned are the 2-hydroxyethyl and the 3-hydroxypropyl radicals.

1-4C-Alkoxycarbonyl represents a radical which, in addition to the carbonyl group, contains one of the abovementioned 1-4C-alkoxy radicals. Examples which may be mentioned are the methoxycarbonyl, the ethoxycarbonyl and the isopropoxycarbonyl radicals.

Halogen within the meaning of the invention is bromine, chlorine or fluorine.

Pyridinyl-1-4C-alkyl represents one of the abovementioned 1-4C-alkyl radicals, which is substituted by a pyridyl radical. Examples which may be mentioned are the pyridylmethyl, the 2-pyridylethyl and the 3-pyridylpropyl radicals.

Pyridinyl or pyridyl includes pyridin-2-yl, pyridin-3-yl and pyridin-4-yl.

Aryl-1-4C-alkyl represents one of the abovementioned 1-4C-alkyl radicals, which is substituted by an aryl radical. Examples which may be mentioned are the arylmethyl, the 2-arylethyl and the 3-arylpropyl radicals.

Aryl stands for R205- and/or R206-substituted phenyl.

Mono- or di-1-4C-alkoxy-2-4C-alkyl represents 2-4C-alkyl radicals, which are substituted by one or two of the abovementioned 1-4C-alkoxy radicals. Examples which may be mentioned are the methoxyethyl, ethoxyethyl and the isopropoxyethyl radicals, particularly the 2-methoxyethyl, 2-ethoxyethyl and the 2-isopropoxyethyl radicals, as well as the dimethoxy-ethyl and the diethoxy-ethyl radicals, particularly the 2,2-dimethoxy-ethyl and the 2,2-diethoxy-ethyl radicals.

Mono- or di-1-4C-alkoxycarbonyl-1-4C-alkyl represents one of the abovementioned 1-4C-alkyl radicals, which is substituted by one or two of the abovementioned 1-4C-alkoxycarbonyl radicals. Examples which may be mentioned are the methoxycarbonylmethyl, the 2-methoxycarbonylethyl and the 1,2-(dimethoxycarbonyl)-ethyl radicals.

Each of the radicals Het1, Het2, Het3, Het4, Het5 and Het7 is optionally substituted as indicated above, and represents independently a 3- to 7-membered fully saturated monocyclic heterocyclic ring radical comprising one nitrogen atom as indicated above and optionally one further heteroatom selected from the group consisting of oxygen, nitrogen and sulfur.

Het1, Het2, Het3, Het4, Het5 and Het7 may include independently, without being restricted thereto, aziridinyl, azetidinyl, pyrrolidinyl, piperidinyl, homopiperidinyl, morpholinyl, thiomorpholinyl, oxazolidinyl, isoxazolidinyl, thiazolidinyl, isothiazolidinyl, pyrazolidinyl, imidazolidinyl, piperazinyl or homopiperazinyl.

As further examples for Het1, Het2, Het3, Het4, Het5 or Het7 according to this invention may be mentioned, without being restricted thereto, derivatives of the abovementioned exemplary radicals which are substituted by a substituent as indicated above, notably, for example, those radicals, which are substituted on a ring nitrogen atom by a substituent as indicated above, such as, as example for Het2, 4N-(R10)-piperazinyl or 4N-(R10)-homopiperazinyl, or, as example for Het7, 4N-(R181)-piperazinyl or 4-N-(R181)-homopiperazinyl.

Illustratively, a suitable example for Het1, Het2, Het3, Het4, Het5 and Het7 radicals include, for example, without being restricted thereto, morpholin-4-yl. Further suitable examples include for Het2, without being restricted thereto, 4N-(R10)-piperazin-1-yl, and for Het7, without being restricted thereto, 4-N-(R181)-piperazin-1-yl.

Het6 is optionally substituted by R203 and/or R204 and stands for a monocylic 3- to 7-membered fully saturated heterocyclic ring radical comprising one to three heteroatoms, each of which is selected from the group consisting of nitrogen, oxygen and sulfur.

In particular, Het6 is optionally substituted by R203 and/or R204 and refers within the meaning of this invention, in a special facet (facet 1) according to the present invention, to a monocyclic 3- to 7-membered fully saturated heterocyclic ring radical comprising one nitrogen atom and optionally one further heteroatom selected from the group consisting of oxygen, nitrogen and sulfur.

More precisely, within the context of this invention, Het6 can be bonded to the carbonyl moiety of —C(O)R20, in one facet (facet 1a) of this invention, via a ring carbon atom or, in another facet (facet 1a′), via a ring nitrogen atom.

Yet more precisely, Het6 is optionally substituted by R203 and/or R204 on a ring nitrogen or ring carbon atom.

Het6 may include, without being restricted thereto, aziridinyl, azetidinyl, pyrrolidinyl, piperidinyl, homopiperidinyl, morpholinyl, thiomorpholinyl, oxazolidinyl, isoxazolidinyl, thiazolidinyl, isothiazolidinyl, pyrazolidinyl, imidazolidinyl, piperazinyl or homopiperazinyl.

In detailed example, Het6 may include according to facet 1a, without being restricted thereto, piperazin-2-yl, piperidin-3-yl, morpholin-3-yl or piperidin4-yl.

Furthermore in detailed example, Het6 may include according to facet 1a′, without being restricted thereto, aziridin-1-yl, azetidin-1-yl, pyrrolidin-1-yl, piperidin-1-yl, homopiperidin-1-yl, pyrazolidin-1-yl, piperazin-1-yl, homopiperazin-1-yl, morpholin-4-yl or thiomorpholin-4-yl.

As further examples for Het6 according to this invention may be mentioned, without being restricted thereto, R203- and/or R204-substituted derivatives of the abovementioned exemplary Het6 radicals, such as, for example according to facet 1a, 1-N-(R203)-4-N-(R204)-piperazin-2-yl, or according to facet 1a′, 4-N-(R203)-piperazin-1-yl.

Illustratively, as exemplary suitable Het6 radicals may be mentioned, for example, without being restricted thereto, morpholin4-yl or 1-N-(R203)4-N-(R204)-piperazin-2-yl.

Har1 is optionally substituted by R91 and/or R92, and is a 5- to 10-membered monocylic or fused bicyclic unsaturated (heteroaromatic) heteroaryl radical comprising 1 to 4 heteroatoms selected independently from the group consisting of oxygen, nitrogen and sulfur.

It is to be understood that the radical Har1 is bonded to the parent molecular group via a ring carbon atom.

Har1 may include, without being restricted thereto, furanyl, thiophenyl, pyrrolyl, oxazolyl, isoxazolyl, thiazolyl, isothiazolyl, imidazolyl, pyrazolyl, triazolyl (precisely: 1,2,4-triazolyl or 1,2,3-triazolyl), thiadiazolyl (precisely: 1,3,4-thiadiazolyl, 1,2,5-thiadiazolyl, 1,2,3-thiadiazolyl or 1,2,4-thiadiazolyl), oxadiazolyl (precisely: 1,3,4-oxadiazolyl, 1,2,5-oxadiazolyl, 1,2,3-oxadiazolyl or 1,2,4-oxadiazolyl) or tetrazolyl; or, pyridinyl, pyrimidinyl, pyrazinyl or pyridazinyl; or the fused or benzofused derivatives of the abovementioned exemplary radicals, such as, for example those mentioned more detailed in the embodimental detail below; as well as the R91- and/or R92-subsfituted derivatives of theses radicals.

In an embodimental detail (detail 1) according to this invention, Har1 is optionally substituted by R91 and/or R92, and is a 9- or 10-membered fused bicyclic unsaturated (heteroaromatic) heteroaryl radical comprising 1 to 4 heteroatoms independently selected from the group consisting of oxygen, nitrogen and sulfur.

Har1 may include according to this detail 1, without being restricted thereto, benzothiophenyl, benzofuranyl, indolyl, benzoxazolyl, benzothiazolyl, indazolyl, benzimidazolyl, benzisoxazolyl, benzisothiazolyl, benzofurazanyl, benzotriazolyl, benzothiadiazolyl, quinolinyl, isoquinolinyl, quinazolinyl, quinoxalinyl, phthalazinyl or cinnolinyl; or indolizinyl, purinyl, naphthyridinyl, imidazopyridinyl or pteridinyl; as well as the R91- and/or R92-substituted derivatives thereof.

Illustratively, as exemplary suitable Har1 radicals according to detail 1 may be mentioned, for example, without being restricted thereto, quinolinyl, naphthyridinyl or imidazopyridinyl, as well as the R91- and/or R92-substituted derivafives thereof.

As more specific exemplary suitable Har1 radicals according to detail 1 may be mentioned, for example, without being restricted thereto, quinolin-3-yl, 2,3-dimethyl-imidazo[1,2-a]pyridin-7-yl or [1,7]naphthyridin-8-yl.

In a further embodimental detail (detail 2) according to this invention, Har1 is optionally substituted by R91 and/or R92, and is a 6-membered monocyclic unsaturated (heteroaromatic) heteroaryl radical comprising one or two nitrogen atoms.

Har1 may include according to this detail 2, without being restricted thereto, pyridinyl, pyrimidinyl, pyrazinyl or pyridazinyl; as well as the R91- and/or R92-substituted derivatives thereof.

Illustratively, as exemplary suitable Har1 radicals according to detail 2 may be mentioned, for example, without being restricted thereto, pyridinyl, as well as the R91- and/or R92-substituted derivatives thereof.

As more specific exemplary suitable Har1 radicals according to detail 2 may be mentioned, for example, without being restricted thereto, dimethoxypyridinyl, such as, for example, 2,6-dimethoxypyridin-4-yl or, in particular, 2,6-dimethoxypyridin-3-yl.

Har2 is optionally substituted by R201 and/or R202, and is a 5 to 10-membered monocylic or fused bicyclic unsaturated (heteroaromatic) heteroaryl radical comprising 1 to 4 heteroatoms selected independently from the group consisting of oxygen, nitrogen and sulfur.

Preferably, the radical Har2 is bonded to the parent molecular group via a ring carbon atom.

Har2 may include, without being restricted thereto, furanyl, thiophenyl, pyrrolyl, oxazolyl, isoxazolyl, thiazolyl, isothiazolyl, imidazolyl, pyrazolyl, triazolyl (precisely: 1,2,4-triazolyl or 1,2,3-triazolyl), thiadiazolyl (precisely: 1,3,4-thiadiazolyl, 1,2,5-thiadiazolyl, 1,2,3-thiadiazolyl or 1,2,4-thiadiazolyl), oxadiazolyl (precisely: 1,3,4-oxadiazolyl, 1,2,5-oxadiazolyl, 1,2,3-oxadiazolyl or 1,2,4-oxadiazolyl) or tetrazolyl; or, pyridinyl, pyrimidinyl, pyrazinyl or pyridazinyl; or the fused or benzofused or pyridofused derivatives of the abovementioned exemplary radicals; as well as the R201- and/or R202-substituted derivatives of theses radicals.

In an embodimental detail according to this invention, Har2 is optionally substituted by R201 and/or R202, and is a 6-membered monocyclic unsaturated (heteroaromatic) heteroaryl radical comprising one or two nitrogen atoms.

Har2 may include according to this detail, without being restricted thereto, pyridinyl, pyrimidinyl, pyrazinyl or pyridazinyl; as well as the R201- and/or R202-substituted derivatives thereof.

Illustratively, as exemplary suitable Har2 radical may be mentioned, for example, without being restricted thereto, pyridinyl.

As more specific exemplary suitable Har2 radicals may be mentioned, for example, without being restricted thereto, pyridin-3-yl or pyridin-4-yl.

The heterocyclic groups mentioned herein refer, unless otherwise mentioned, to all of the possible isomeric forms thereof.

The heterocyclic groups mentioned herein refer, unless otherwise noted, in particular to all of the possible positional isomers thereof.

Thus, for example, the term pyridyl or pyridinyl includes pyridin-2-yl, pyridin-3-yl and pyridin-4-yl.

The heterocyclic groups mentioned herein refer, unless otherwise noted, also to all of the possible tautomers thereof, in pure form as well as any mixtures thereof.

Constituents which are optionally substituted as stated herein, may be substituted, unless otherwise noted, at any possible position.

The heterocyclic groups, alone or as part of other groups, mentioned herein may be substituted by their given substituents, unless otherwise noted, at any possible position, such as e.g. at any substitutable ring carbon or ring nitrogen atom.

Unless otherwise noted, rings containing quaternizable imino-type ring nitrogen atoms (—N═) may be preferably not quaternized on these imino-type ring nitrogen atoms by the mentioned substituents or parent molecular groups.

Unless otherwise noted, any heteroatom of a heterocyclic ring with unsatisfied valences mentioned herein is assumed to have the hydrogen atom(s) to satisfy the valences.

When any variable occurs more than one time in any constituent, each definition is independent.

As it is known for the person skilled in the art, compounds comprising nitrogen atoms can form N-oxides.

Particularly, imine nitrogen, especially heterocyclic or heteroaromatic imine nitrogen, or pyridine-type nitrogen (═N—) atoms, can be N-oxidized to form the N-oxides comprising the group ═N⁺(O⁻)—. Thus, the compounds according to the present invention comprising the imine nitrogen atom in position 5 of the phenylphenanthridine backbone and, optionally (depending on the meaning of R7), one or more further nitrogen atoms suitable to exist in the N-oxide state (═N⁺(O⁻)—) may be capable to form (depending on the number of nitrogen atoms suitable to form stabile N-oxides) mono-N-oxides, bis-N-oxides or multi-N-oxides, or mixtures thereof.

The term N-oxide(s) as used in this invention therefore encompasses all possible, and in particular all stabile, N-oxide forms, such as mono-N-oxides, bis-N-oxides or multi-N-oxides, or mixtures thereof in any mixing ratio.

Possible salts for compounds of the formula I—depending on substitution—are all acid addition salts or all salts with bases. Particular mention may be made of the pharmacologically tolerable salts of the inorganic and organic acids and bases customarily used in pharmacy. Those suitable are, on the one hand, water-insoluble and, particularly, water-soluble acid addition salts with acids such as, for example, hydrochloric acid, hydrobromic acid, phosphoric acid, nitric acid, sulfuric acid, acetic acid, citric acid, Dgluconic acid, benzoic acid, 2-(4-hydroxybenzoyl)benzoic acid, butyric acid, sulfosalicylic acid, maleic acid, lauric acid, malic acid, fumaric acid, succinic acid, oxalic acid, tartaric acid, embonic acid, stearic acid, toluenesulfonic acid, methanesulfonic acid or 3-hydroxy-2-naphthoic acid, it being possible to employ the acids in salt preparation—depending on whether a mono- or polybasic acid is concerned and depending on which salt is desired—in an equimolar quantitative ratio or one differing therefrom.

On the other hand, salts with bases are also suitable. Examples of salts with bases which may be mentioned are alkali metal (lithium, sodium, potassium) or calcium, aluminum, magnesium, titanium, ammonium, meglumine or guanidinium salts, where here too the bases are employed in salt preparation in an equimolar quantitative ratio or one differing therefrom.

Pharmacologically intolerable salts which can initially be obtained, for example, as process products in the preparation of the compounds according to the invention on an industrial scale are converted into pharmacologically tolerable salts by processes known to the person skilled in the art.

It is known to the person skilled in the art that the compounds according to the invention and their salts, when they are isolated, for example, in crystalline form, can contain various amounts of solvents. The invention therefore also comprises all solvates and in particular all hydrates of the compounds of the formula I, and also all solvates and in particular all hydrates of the salts of the compounds of the formula I.

The substituents R6 and —C(O)R7 of compounds of formula I can be attached in the ortho, meta or para position with respect to the binding position in which the 6-phenyl ring is bonded to the phenanthridine ring system, whereby preference is given to the attachement of —C(O)R7 in the meta or in the para position. In another embodiment preference is given to the attachement of —C(O)R7 in the meta or in the para position, and R6 is hydrogen. In yet another embodiment preference is given to the attachement of —C(O)R7 in the meta position, and R6 is hydrogen. In still yet another embodiment preference is given to the attachement of —C(O)R7 in the para position, and R6 is hydrogen.

Compounds of formula I to be more worthy to be mentioned are those in which
R1 is 1-2C-alkoxy, 3-5C-cycloalkoxy, 3-5C-cycloalkylmethoxy, 2,2-difluoroethoxy, or completely or predominantly fluorine-substituted 1-2C-al koxy,
R2 is 1-2C-alkoxy, 3-5C-cycloalkoxy, 3-5C-cycloalkylmethoxy, 2,2-difluoroethoxy, or completely or predominantly fluorine-substituted 1-2C-al koxy,
R3 is hydrogen,
R31 is hydrogen,
   either, in a first embodiment (embodiment a) according to the present invention,
R4 is —O—R41, in which
R41 is hydrogen or 1-4C-alkylcarbonyl, and
R5 is hydrogen,
   or, in a second embodiment (embodiment b) according to the present invention,
R4 is hydrogen, and
R5 is —O—R51, in which
R51 is hydrogen or 1-4C-alkylcarbonyl,
R6 is hydrogen,
   either,
   in a first aspect (aspect 1) according to the present invention,
R7 is —N(R8)R9, in which
R8 is hydrogen, 1-4C-alkyl or 1-4C-alkoxy-2-4C-alkyl,
R9 is hydrogen, 1-C-alkyl, mono- or di-1-4C-alkoxy-2-4C-alkyl, hydroxy-2-4C-alkyl, mono- or di-1-4C-alkoxycarbonyl-1-4C-alkyl, Har1, pyridinyl-1-4C-alkyl, 3-7C-cycloalkyl, or 2-4C-alkyl substituted by —NR(93)R94, in which
Har1 is optionally substituted by R91 and/or R92, and is a 5 to 10-membered monocylic or fused bicyclic unsaturated heteroaryl radical comprising 1 to 4 hete roatoms selected independently from the group consisting of oxygen, nitrogen and sulfur, in which
R91 is 1-4C-alkyl or 1-4C-alkoxy,
R92 is 1-4C-alkyl or 1-4C-alkoxy,
R93 is hydrogen or 1-4C-alkyl,
R94 is hydrogen or 1-4C-alkyl,
   or R93 and R94 together and with inclusion of the nitrogen atom, to which they are attached, form a heterocyclic ring Het1, in which
Het1 is optionally substituted by R931, and is a 3- to 7-membered saturated monocyclic heterocyclic ring radical comprising the nitrogen atom, to which R93 and R94 are bonded, and optionally one further heteroatom selected from the group consisting of oxygen, nitrogen and sulfur, in which
R931 is 1-4C-alkyl,
   or R8 and R9 together and with inclusion of the nitrogen atom, to which they are attached, form a heterocyclic ring Het2, in which
Het2 is optionally substituted by R10, and is a 3- to 7-membered saturated monocyclic heterocyclic ring radical comprising the nitrogen atom, to which R8 and R9 are bonded, and optionally one further heteroatom selected from the group consisting of oxygen, nitrogen and sulfur, in which
R10 is 1-4C-alkyl, —C(O)R11, pyridyl, 2-4C-alkyl substituted by —NR(14)R15, or 1-4C-alkyl substituted by —C(O)N(R16)R17, in which
R11 is 1-4C-alkyl substituted by —NR(12)R13, in which
R12 is hydrogen or 1-4C-alkyl,
R13 is hydrogen or 1-4C-alkyl,
   or R12 and R13 together and with inclusion of the nitrogen atom, to which they are attached, form a heterocyclic ring Het3, in which
Het3 is optionally substituted by R121, and is a 3- to 7-membered saturated monocyclic heterocyclic ring radical comprising the nitrogen atom, to which R12 and R13 are bonded, and optionally one further heteroatom selected from the group consisting of oxygen, nitrogen and sulfur, in which
R121 is 1-4C-alkyl,
R14 is hydrogen or 1-4C-alkyl,
R15 is hydrogen or 1-4C-alkyl,
   or R14 and R15 together and with inclusion of the nitrogen atom, to which they are attached, form a heterocyclic ring Het4, in which
Het4 is optionally substituted by R141, and is a 3- to 7-membered saturated monocyclic heterocyclic ring radical comprising the nitrogen atom, to which R14 and R15 are bonded, and optionally one further heteroatom selected from the group consisting of oxygen, nitrogen and sulfur, in which
R141 is 1-4C-alkyl,
R16 is hydrogen, 1-4C-alkyl or pyridyl,
R17 is hydrogen or 1-4C-alkyl,
   or R16 and R17 together and with inclusion of the nitrogen atom, to which they are attached, form a heterocyclic ring Het5, in which
Het5 is optionally substituted by R161, and is a 3- to 7-membered saturated monocyclic heterocyclic ring radical comprising the nitrogen atom, to which R16 and R17 are bonded, and optionally one further heteroatom selected from the group consisting of oxygen, nitrogen and sulfur, in which
R141 is 1-4C-alkyl,
   or,
   in a second aspect (aspect 2) according to the present invention,
R7 is —NH—N(R18)R19, in which
R18 is hydrogen,
R19 is —C(O)R20, or R21-substituted phenyl, in which
R20 is Har2, Het6, or Aryl-1-4C-alkyl, in which
Har2 is optionally substituted by R201 and/or R202, and is a 5- to 10-membered monocylic or fused bicyclic unsaturated heteroaryl radical comprising 1 to 4 heteroatoms selected independently from the group consisting of oxygen, nitrogen and sulfur, in which
R201 is 1-4C-alkyl or 1-4C-alkoxy,
R202 is 1-4C-alkyl or 1-4C-alkoxy,
Het6 is optionally substituted by R203 and/or R204, and is a monocylic 3- to 7-membered saturated heterocyclic ring radical comprising one to three heteroatoms, each of which is selected from the group consisting of nitrogen, oxygen and sulfur, in which
R203 is 1-4C-alkyl,
R204 is 1-4C-alkyl,
Aryl is R205- and/or R206-substituted phenyl,
R205 is 1-4C-alkoxy
R206 is 1-4C-alkoxy
R21 is aminosulphonyl,
   or R18 and R19 together and with inclusion of the nitrogen atom, to which they are attached, form a heterocyclic ring Het7, in which
Het7 is optionally substituted by R181, and is a 3- to 7-membered saturated monocyclic heterocyclic ring radical comprising the nitrogen atom, to which R18 and R19 are bonded, and optionally one further heteroatom selected from the group consisting of oxygen, nitrogen and sulfur, in which
R181 is 1-4C-alkyl,
   and the salts, the N-oxides and the salts of the N-oxides of these compounds.

Compounds of formula I in particular worthy to be mentioned are those in which
R1 is 1-2C-alkoxy, 2,2-difluoroethoxy, or completely or predominantly fluorine-substituted 1-2C-alkoxy,
R2 is 1-2C-alkoxy, 2,2-difluoroethoxy, or completely or predominantly fluorine-substituted 1-2C-alkoxy,
R3 is hydrogen,
R31 is hydrogen,
R4 is —O—R41, in which
R41 is hydrogen or 1-4C-alkylcarbonyl,
R5 is hydrogen,
R6 is hydrogen,
   either,
   in a first aspect (aspect 1) according to the present invention,
R7 is —N(R8)R9, in which
R8 is hydrogen, 1-4C-alkyl or 1-4C-alkoxy-2-4C-alkyl,
R9 is hydrogen, 1-4C-alkyl, mono- or di-1-4C-alkoxy-2-4C-alkyl, hydroxy-2-4C-alkyl, mono- or di-1-4C-alkoxycarbonyl-1-4C-alkyl, Har1, pyridinyl-1-4C-alkyl, 3-7C-cycloalkyl, or 2-4C-alkyl substituted by —NR(93)R94, in which
   either
Har1 is optionally substituted by R91 and/or R92, and is a 9- or 10-membered fused bicyclic unsaturated heteroaryl radical comprising 1 to 4 heteroatoms selected independently from the group consisting of oxygen, nitrogen and sulfur, in which
R91 is 1-4C-alkyl,
R92 is 1-4C-alkyl,
   or
Har1 is optionally substituted by R91 and/or R92, and is a 6-membered monocyclic unsaturated heteroaryl radical comprising one or two nitrogen atoms, in which
R91 is 1-4C-alkoxy,
R92 is 1-4C-alkoxy,
R93 is hydrogen or 1-4C-alkyl,
R94 is hydrogen or 1-4C-alkyl,
   or R93 and R94 together and with inclusion of the nitrogen atom, to which they are attached, form a heterocyclic ring Het1, in which
Het1 is optionally substituted by R931, and is a 3- to 7-membered saturated monocyclic heterocyclic ring radical comprising the nitrogen atom, to which R93 and R94 are bonded, and optionally one further heteroatom selected from the group consisting of oxygen, nitrogen and sulfur, in which
R931 is 1-4C-alkyl,
   or R8 and R9 together and with inclusion of the nitrogen atom, to which they are attached, form a heterocyclic ring Het2, in which
Het2 is optionally substituted by R10, and is a 3- to 7-membered saturated monocyclic heterocyclic ring radical comprising the nitrogen atom, to which R8 and R9 are bonded, and optionally one further heteroatom selected from the group consisting of oxygen, nitrogen and sulfur, in which
R10 is 1-4C-alkyl, —C(O)R11, pyridyl, 2-4C-alkyl substituted by —NR(14)R15, or 1-4C-alkyl substituted by —C(O)N(R16)R17, in which
R11 is 1-4C-alkyl substituted by —NR(12)R13, in which
R12 is hydrogen or 1-4C-alkyl,
R13 is hydrogen or 1-4C-alkyl,
   or R12 and R13 together and with inclusion of the nitrogen atom, to which they are attached, form a heterocyclic ring Het3, in which
Het3 is optionally substituted by R121, and is a 3- to 7-membered saturated monocyclic heterocyclic ring radical comprising the nitrogen atom, to which R12 and R13 are bonded, and optionally one further heteroatom selected from the group consisting of oxygen, nitrogen and sulfur, in which
R121 is 1-4C-alkyl,
R14 is hydrogen or 1-4C-alkyl,
R15 is hydrogen or 1-4C-alkyl,
   or R14 and R15 together and with inclusion of the nitrogen atom, to which they are attached, form a heterocyclic ring Het4, in which
Het4 is optionally substituted by R141, and is a 3- to 7-membered saturated monocyclic heterocyclic ring radical comprising the nitrogen atom, to which R14 and R15 are bonded, and optionally one further heteroatom selected from the group consisting of oxygen, nitrogen and sulfur, in which
R141 is 1-4C-alkyl,
R16 is hydrogen, 1-4C-alkyl or pyridyl,
R17 is hydrogen or 1-4C-alkyl,
   or R16 and R17 together and with inclusion of the nitrogen atom, to which they are attached, form a heterocyclic ring Het5, in which
Het5 is optionally substituted by R161, and is a 3- to 7-membered saturated monocyclic heterocyclic ring radical comprising the nitrogen atom, to which R16 and R17 are bonded, and optionally one further heteroatom selected from the group consisting of oxygen, nitrogen and sulfur, in which
R141 is 1-4C-alkyl,
   or,
   in a second aspect (aspect 2) according to the present invention,
R7 is —NH—N(R18)R19, in which
R18 is hydrogen,
R19 is —C(O)R20, or R21-substituted phenyl, in which
R20 is Har2, Het6, or Aryl-1-4C-alkyl, in which
Har2 is a 6-membered monocylic unsaturated heteroaryl radical comprising one or two nitrogen atoms,
Het6 is optionally substituted by R203 and/or R204, and is a monocylic 3- to 7-membered saturated heterocyclic ring radical comprising one to three heteroatoms, each of which is selected from the group consisting of nitrogen, oxygen and sulfur, in which
R203 is 1-4C-alkyl,
R204 is 1-4C-alkyl,
Aryl is R205- and/or R206-substituted phenyl,
R205 is 1-4C-alkoxy
R206 is 1-4C-alkoxy
R21 is aminosulphonyl,
   or R18 and R19 together and with inclusion of the nitrogen atom, to which they are attached, form a heterocyclic ring Het7, in which
Het7 is optionally substituted by R181, and is a 3- to 7-membered saturated monocyclic heterocyclic ring radical comprising the nitrogen atom, to which R18 and R19 are bonded, and optionally one further heteroatom selected from the group consisting of oxygen, nitrogen and sulfur, in which
R181 is 1-4C-alkyl,
   and the salts, the N-oxides and the salts of the N-oxides of these compounds.

Compounds of formula I in more particular worthy to be mentioned are those in which
R1 is 1-2C-alkoxy, 2,2-difluoroethoxy, or completely or predominantly fluorine-substituted 1-2C-alkoxy,
R2 is 1-2C-alkoxy, 2,2-difluoroethoxy, or completely or predominantly fluorine-substituted 1-2C-alkoxy,
R3 is hydrogen,
R31 is hydrogen,
R4 is —O—R41, in which
R41 is hydrogen,
R5 is hydrogen,
R6 is hydrogen,
   either,
   in a first aspect (aspect 1) according to the present invention,
R7 is —N(R8)R9, in which
R8 is hydrogen, 1-C-alkyl or 1-4C-alkoxy-2-4C-alkyl,
R9 is 1-4C-alkyl, mono- or di-1-4C-alkoxy-2-4C-alkyl, hydroxy-2-4C-alkyl, mono- or di-1-2C-alkoxycarbonyl-1-4C-alkyl, Har1, pyridinyl-1-4C-alkyl, 3-5C-cycloalkyl, or 2-4C-alkyl substituted by —NR(93)R94, in which
Har1 is 2,6-dimethoxypyridinyl, quinolinyl, 2,3-dimethyl-imidazo[1,2-a]pyridinyl or [1,7]naphthyridinyl,
R93 and R94 together and with inclusion of the nitrogen atom, to which they are attached, form a heterocyclic ring Het1, in which
Het1 is morpholinyl,
   or R8 and R9 together and with inclusion of the nitrogen atom, to which they are attached, form a haterocyclic ring Het2, in which
Het2 is pyrrolidinyl, morpholinyl or 4N—(R10)-piperazinyl, in which
R10 is —C(O)R11, pyridyl, 2-4C-alkyl substituted by —NR(14)R15, or 1-4C-alkyl substituted by —C(O)N(R16)R17, in which
R11 is 1-4C-alkyl substituted by —NR(12)R13, in which
R12 is 1-4C-alkyl,
R13 is 1-4C-alkyl,
   or R12 and R13 together and with inclusion of the nitrogen atom, to which they are attached, form a heterocyclic ring Het3, in which
Het3 is morpholinyl,
R14 is 1-4C-alkyl,
R15 is 1-4C-alkyl,
   or R14 and R15 together and with inclusion of the nitrogen atom, to which they are attached, form a heterocyclic ring Het4, in which
Het4 is morpholinyl,
R16 is 1-4C-alkyl or pyridyl,
R17 is hydrogen or 1-4C-alkyl,
   or R16 and R17 together and with inclusion of the nitrogen atom, to which they are attached, form a heterocyclic ring Het5, in which
Het5 is pyrrolidinyl or morpholinyl,
   or,
   in a second aspect (aspect 2) according to the present invention,
R7 is —NH—N(R18)R19, in which
R18 is hydrogen,
R19 is —C(O)R20, or R21-substituted phenyl, in which
R20 is pyridinyl, morpholinyl, 1N—(R203)4N—(R204)-piperazinyl, or Aryl-1-2C-alkyl, in which
R203 is 1-4C-alkyl,
R204 is 1-4C-alkyl,
Aryl is 3,4-dimethoxyphenyl,
R21 is aminosulphonyl,
   or R18 and R19 together and with inclusion of the nitrogen atom, to which they are attached, form a heterocyclic ring Het7, in which
Het7 is morpholinyl or 4N—(R181)-piperazinyl, in which
R181 is 1-4C-alkyl,
   and the salts, the N-oxides and the salts of the N-oxides of these compounds.

In another embodiment, compounds of formula I in more particular worthy to be mentioned are those in which
R1 is 1-2C-alkoxy, 2,2-difluoroethoxy, or completely or predominantly fluorine-substituted 1-2C-alkoxy,
R2 is 1-2C-alkoxy, 2,2-difluoroethoxy, or completely or predominantly fluorine-substituted 1-2C-alkoxy,
R3 is hydrogen,
R31 is hydrogen,
R4 is —O—R41, in which
R41 is hydrogen,
R5 is hydrogen,
R6 is hydrogen,
   either,
   in a first aspect (aspect 1) according to the present invention,
R7 is —N(R8)R9, in which
R8 is hydrogen, 1-4C-alkyl or 1-4C-alkoxy-2-4C-alkyl,
R9 is mono- or di-1-4C-alkoxy-2-4C-alkyl, hydroxy-2-4C-alkyl, mono- or di-1-2C-alkoxycarbonyl-1-4C-alkyl, Har1, pyridinyl-1-4C-alkyl, or 2-4C-alkyl substituted by —NR(93)R94, in which
Har1 is 2,6-dimethoxypyridinyl, quinolinyl, 2,3-dimethyl-imidazo[1,2-a]pyridinyl or [1,7]naphthyridinyl,
R93 and R94 together and with inclusion of the nitrogen atom, to which they are attached, form a heterocyclic ring Het1, in which
Het1 is morpholinyl,
   or R8 and R9 together and with inclusion of the nitrogen atom, to which they are attached, form a heterocyclic ring Het2, in which
Het2 is 4N—(R10)-piperazinyl, in which
R10 is —C(O)R11, pyridyl, 2-4C-alkyl substituted by —NR(14)R15, or 1-4C-alkyl substituted by —C(O)N(R16)R17, in which
R11 is 1-4C-alkyl substituted by —NR(12)R13, in which
R12 is 1-4C-alkyl,
R13 is 1-4C-alkyl,
   or R12 and R13 together and with inclusion of the nitrogen atom, to which they are attached, form a heterocyclic ring Het3, in which
Het3 is morpholinyl,
R14 is 1-4C-alkyl,
R15 is 1-4C-alkyl,
   or R14 and R15 together and with inclusion of the nitrogen atom, to which they are attached, form a heterocyclic ring Het4, in which
Het4 is morpholinyl,
R16 is 1-4C-alkyl or pyridyl,
R17 is hydrogen or 1-4C-alkyl,
   or R16 and R17 together and with inclusion of the nitrogen atom, to which they are attached, form a heterocyclic ring Het5, in which
Het5 is pyrrolidinyl or morpholinyl,
   or,
   in a second aspect (aspect 2) according to the present invention,
R7 is —NH—N(R18)R19, in which
R18 is hydrogen,
R19 is —C(O)R20, or R21-substituted phenyl, in which
R20 is pyridinyl, morpholinyl, 1N—(R203)-4N—(R204)-piperazinyl, or Aryl-1-2C-alkyl, in which
R203 is 1-4C-alkyl,
R204 is 1-4C-alkyl,
Aryl is 3,4-dimethoxyphenyl,
R21 is aminosulphonyl,
   or R18 and R19 together and with inclusion of the nitrogen atom, to which they are attached, form a heterocyclic ring Het7, in which
Het7 is morpholinyl or 4N—(R181)-piperazinyl, in which
R181 is 1-4C-alkyl,
   the enantiomers, as well as the salts, the N-oxides and the salts of the N-oxides of these compounds and enantiomers.

In yet another embodiment, compounds of formula I in more particular worthy to be mentioned are those in which
R1 is 1-2C-alkoxy, 2,2-difluoroethoxy, or completely or predominantly fluorine-substituted 1-2C-alkoxy,
R2 is 1-2C-alkoxy, 2,2-difluoroethoxy, or completely or predominantly fluorine-substituted 1-2C-alkoxy,
R3 is hydrogen,
R31 is hydrogen,
R4 is —O—R41, in which
R41 is hydrogen,
R5 is hydrogen,
R6 is hydrogen,
R7 is —N(R8)R9, in which
R8 is hydrogen or 1-4C-alkyl,
R9 is 1-4C-alkyl or 3-5C-cycloalkyl,
   the enantiomers, as well as the salts, the N-oxides and the salts of the N-oxides of these compounds and enantiomers.

In still yet another embodiment, compounds of formula I in more particular worthy to be mentioned are those in which
R1 is 1-2C-alkoxy, 2,2-difluoroethoxy, or completely or predominantly fluorine-substituted 1-2C-alkoxy,
R2 is 1-2C-alkoxy, 2,2-difluoroethoxy, or completely or predominantly fluorine-substituted 1-2C-alkoxy,
R3 is hydrogen,
R31 is hydrogen,
R4 is —O—R41, in which
R41 is hydrogen,
R5 is hydrogen,
R6 is hydrogen,
R7 is —N(R8)R9, in which
R8 is hydrogen or 1-4C-alkyl,
R9 is Har1, in which
Har1 is substituted by R91 and R92, and is pyridinyl, in which
R91 is 1-4C-alkoxy,
R92 is 1-4C-alkoxy,
   the enantiomers, as well as the salts, the Noxides and the salts of the Noxides of these compounds and enantiomers.

Compounds of formula I in further more particular worthy to be mentioned are those in which
R1 is 1-2C-alkoxy, 2,2-difluoroethoxy, or completely or predominantly fluorine-substituted 1-2C-alkoxy,
R2 is 1-2C-alkoxy, 2,2-difluoroethoxy, or completely or predominantly fluorine-substituted 1-2C-alkoxy,
R3 is hydrogen,
R31 is hydrogen,
R4 is —O—R41, in which
R41 is hydrogen,
R5 is hydrogen,
R6 is hydrogen,
   either,
   in a first aspect (aspect 1) according to the present invention,
R7 is —N(R8)R9, in which
R8 is hydrogen, methyl, ethyl or 2-methoxyethyl,
R9 is methyl, 2-methoxyethyl, methoxycarbonylmethyl, 1,2-di-(methoxycarbonyl)-ethyl, Har1, 2-pyridinyl-ethyl, cyclopropyl, or 2-3C-alkyl substituted by —NR(93)R94, in which
Har1 is 2,6-dimethoxypyridinyl, quinolinyl, 2,3-dimethyl-imidazo[1,2-a]pyridinyl or [1,7]naphthyridinyl,
R93 and R94 together and with inclusion of the nitrogen atom, to which they are attached, form a heterocyclic ring Het1, in which
Het1 is morpholinyl,
   or R8 and R9 together and with inclusion of the nitrogen atom, to which they are attached, form a heterocyclic ring Het2, in which
Het2 is pyrrolidinyl, morpholinyl or 4N—(R10)-piperazinyl, in which
R10 is pyridyl, ethyl substituted by —NR(14)R15, or methyl substituted by —C(O)N(R16)R17, in which
R14 is methyl,
R15 is methyl,
   or R14 and R15 together and with inclusion of the nitrogen atom, to which they are attached, form a heterocyclic ring Het4, in which
Het4 is morpholinyl,
R16 is methyl or pyridyl,
R17 is hydrogen or methyl,
   or R16 and R17 together and with inclusion of the nitrogen atom, to which they are attached, form a heterocyclic ring Het5, in which
Het5 is pyrrolidinyl or morpholinyl,
   or,
   in a second aspect (aspect 2) according to the present invention,
R7 is —NH—N(R18) R19, in which
R18 is hydrogen,
R19 is —C(O)R20, or R21-substituted phenyl, in which
R20 is pyridinyl, or morpholin-4-yl,
R21 is aminosulphonyl,
   or R18 and R19 together and with inclusion of the nitrogen atom, to which they are attached, form a heterocyclic ring Het7, in which
Het7 is morpholinyl or 4N—(R181)-piperazinyl, in which
R181 is methyl,
   and the salts, the N-oxides and the salts of the N-oxides of these compounds.

Compounds of formula I in still further more particular worthy to be mentioned are those in which one of R1 and R2 is methoxy, and the other is methoxy, ethoxy, 2,2-difluoroethoxy, or difluoromethoxy,
R3 is hydrogen,
R31 is hydrogen,
R4 is —O—R41, in which
R41 is hydrogen,
R5 is hydrogen,
R6 is hydrogen,
   either,
   in a first aspect (aspect 1) according to the present invention,
R7 is —N(R8)R9, in which
R8 is hydrogen, methyl, ethyl or 2-methoxyethyl,
R9 is methyl, 2-methoxyethyl, methoxycarbonylmethyl, 1,2-di-(methoxycarbonyl)-ethyl, Har1, 2-pyridinyl-ethyl, cyclopropyl, or 2-3C-alkyl substituted by —NR(93)R94, in which Har1 is 2,6-dimethoxypyridinyl, quinolinyl, 2,3-dimethyl-imidazo[1,2-a]pyridinyl or [1,7]naphthyridinyl,
R93 and R94 together and with inclusion of the nitrogen atom, to which they are attached, form a heterocyclic ring Het1, in which
Het1 is morpholinyl,
   or R8 and R9 together and with inclusion of the nitrogen atom, to which they are attached, form a heterocyclic ring Het2, in which
Het2 is pyrrolidinyl, morpholinyl or 4N—(R10)-piperazinyl, in which
R10 is pyridyl, ethyl substituted by —NR(14)R15, or methyl substituted by —C(O)N(R16)R17, in which
R14 is methyl,
R15 is methyl,
   or R14 and R15 together and with inclusion of the nitrogen atom, to which they are attached, form a heterocyclic ring Het4, in which
Het4 is morpholinyl,
R16 is methyl or pyridyl,
R17 is hydrogen or methyl,
   or R16 and R17 together and with inclusion of the nitrogen atom, to which they are attached, form a heterocyclic ring Het5, in which
Het5 is pyrrolidinyl or morpholinyl,
   or,
   in a second aspect (aspect 2) according to the present invention,
R7 is —NH—N(R18)R19, in which
R18 is hydrogen,
R19 is —C(O)R20, or R21-substituted phenyl, in which
R20 is pyridinyl, or morpholin-4-yl,
R21 is aminosulphonyl,
   or R18 and R19 together and with inclusion of the nitrogen atom, to which they are attached, form a heterocyclic ring Het7, in which
Het7 is morpholinyl or 4N—(R181)-piperazinyl, in which
R181 is methyl,
whereby the radical —C(O)R7 is attached in the meta or para position with respect to the binding position in which the phenyl moiety is bonded to the parent molecular group,
   and the salts, the N-oxides and the salts of the N-oxides of these compounds.

In another embodiment, compounds of formula I in further more particular worthy to be mentioned are those in which
R1 is methoxy,
R2 is methoxy, ethoxy, 2,2-difluoroethoxy, or difluoromethoxy,
R3 is hydrogen,
R31 is hydrogen,
R4 is —O—R41, in which
R41 is hydrogen,
R5 is hydrogen,
R6 is hydrogen,
   either,
   in a first aspect (aspect 1) according to the present invention,
R7 is —N(R8)R9, in which
R8 is hydrogen, methyl, ethyl or 2-methoxyethyl,
R9 is 2-methoxyethyl, methoxycarbonylmethyl, 1,2-di-(methoxycarbonyl)-ethyl, Har1, 2-pyridinyl-ethyl, or 2-3C-alkyl substituted by —NR(93)R94, in which
Har1 is 2,6-dimethoxypyridinyl, quinolinyl, 2,3-dimethyl-imidazo[1,2-a]pyridinyl or [1,7]naphthyridinyl,
R93 and R94 together and with inclusion of the nitrogen atom, to which they are attached, form a heterocyclic ring Het1, in which
Het1 is morpholinyl,
   or R8 and R9 together and with inclusion of the nitrogen atom, to which they are attached, form a heterocyclic ring Het2, in which
Het2 is 4N—(R10)-piperazinyl, in which
R10 is pyridyl, ethyl substituted by —NR(14)R15, or methyl substituted by —C(O)N(R16)R17, in which
R14 is methyl,
R15 is methyl,
   or R14 and R15 together and with inclusion of the nitrogen atom, to which they are attached, form a heterocyclic ring Het4, in which
Het4 is morpholinyl,
R16 is methyl or pyridyl,
R17 is hydrogen or methyl,
   or R16 and R17 together and with inclusion of the nitrogen atom, to which they are attached, form a heterocyclic ring Het5, in which
Het5 is pyrrolidinyl or morpholinyl,
   or,
   in a second aspect (aspect 2) according to the present invention,
R7 is —NH—N(R18)R19, in which
R18 is hydrogen,
R19 is —C(O)R20, or R21-substituted phenyl, in which
R20 is pyridinyl, or morpholin-4-yl,
R21 is aminosulphonyl,
   or R18 and R19 together and with inclusion of the nitrogen atom, to which they are attached, form a heterocyclic ring Het7, in which
Het7 is morpholinyl or 4N—(R181)-piperazinyl, in which
R181 is methyl,
whereby the radical —C(O)R7 is attached in the meta or para position with respect to the binding position in which the phenyl moiety is bonded to the parent molecular group,
   the enantiomers, as well as the salts, the N-oxides and the salts of the N-oxides of these compounds and enantiomers.

In yet another embodiment, compounds of formula I in still further more particular worthy to be mentioned are those in which
R1 is methoxy,
R2 is methoxy, ethoxy, 2,2-difluoroethoxy, or difluoromethoxy,
R3 is hydrogen,
R31 is hydrogen,
R4 is —O—R41, in which
R41 is hydrogen,
R5 is hydrogen,
R6 is hydrogen,
R7 is —N(R8)R9, in which
R8 is hydrogen, methyl, ethyl, or isopropyl,
R9 is methyl, ethyl, isopropyl, cyclopropyl or cyclobutyl,
whereby the radical —C(O)R7 is attached in the meta or para position with respect to the binding position in which the phenyl moiety is bonded to the parent molecular group,
   the enantiomers, as well as the salts, the N-oxides and the salts of the N-oxides of these compounds and enantiomers.

In still yet another embodiment, compounds of formula I in still further more particular worthy to be mentioned are those in which
R1 is methoxy,
R2 is methoxy, ethoxy, 2,2-difluoroethoxy, or difluoromethoxy,
R3 is hydrogen,
R31 is hydrogen,
R4 is —O—R41, in which
R41 is hydrogen,
R5 is hydrogen,
R6 is hydrogen,
R7 is —N(R8)R9, in which
R8 is hydrogen or 1-4C-alkyl,
R9 is Har1, in which
Har1 is substituted by R91 and R92, and is pyridinyl, in which
R91 is methoxy or ethoxy,
R92 is methoxy or ethoxy,
whereby the radical —C(O)R7 is attached in the meta or para position with respect to the binding position in which the phenyl moiety is bonded to the parent molecular group,
   the enantiomers, as well as the salts, the Noxides and the salts of the Noxides of these compounds and enantiomers.

Compounds of formula I to be emphasized are those in which
R1 is methoxy,
R2 is methoxy, ethoxy, 2,2-difluoroethoxy, or difluoromethoxy,
R3 is hydrogen,
R31 is hydrogen,
R4 is —O—R41, in which
R41 is hydrogen,
R5 is hydrogen,
R6 is hydrogen,
R7 is —N(R8)R9, in which
   either
R8 is methyl, ethyl or isopropyl, and
R9 is methyl, ethyl or isopropyl,
   or
R8 is hydrogen, and
R9 is cyclopropyl or cyclobutyl,
whereby the radical —C(O)R7 is attached in the meta or para position with respect to the binding position in which the phenyl moiety is bonded to the parent molecular group,
   the enantiomers, as well as the salts, the N-oxides and the salts of the N-oxides of these compounds and enantiomers.

Compounds of formula I to be more emphasized are those in which
R1 is methoxy,
R2 is ethoxy, 2,2-difluoroethoxy, or difluoromethoxy,
R3 is hydrogen,
R31 is hydrogen,
R4 is —O—R41, in which
R41 is hydrogen,
R5 is hydrogen,
R6 is hydrogen,
R7 is —N(R8)R9, in which
R8 is isopropyl,
R9 is isopropyl,
whereby the radical —C(O)R7 is attached in the meta or para position with respect to the binding position in which the phenyl moiety is bonded to the parent molecular group,
   the enantiomers, as well as the salts, the N-oxides and the salts of the N-oxides of these compounds and enantiomers.

Yet compounds of formula I to be more emphasized are those in which
R1 is methoxy,
R2 is ethoxy, 2,2-difluoroethoxy, or difluoromethoxy,
R3 is hydrogen,
R31 is hydrogen,
R4 is —O—R41, in which
R41 is hydrogen,
R5 is hydrogen,
R6 is hydrogen,
R7 is —N(R8)R9, in which
R8 is hydrogen,
R9 is cyclopropyl or cyclobutyl,
whereby the radical —C(O)R7 is attached in the meta or para position with respect to the binding position in which the phenyl moiety is bonded to the parent molecular group,
   the enantiomers, as well as the salts, the N-oxides and the salts of the N-oxides of these compounds and enantiomers.

A special interest in the compounds according to this invention relates to those compounds which are included—within the meaning of this invention—by one or, when possible, by more of the following embodiments:

A special embodiment of the compounds of the present invention include those compounds of formula I in which R1 and R2 are independently 1-2C-alkoxy, 2,2-difluoroethoxy, or completely or predominantly fluorine-substituted 1-2C-alkoxy.

Another special embodiment of the compounds of the present invention include those compounds of formula I in which R1 and R2 are independently 1-20-alkoxy, 2,2-difluoroethoxy, or completely or predominantly fluorine-substituted 1-2C-alkoxy, and R3 and R31 are both hydrogen.

Another special embodiment of the compounds of the present invention include those compounds of formula I in which one of R1 and R2 is methoxy, and the other is methoxy, ethoxy, difluoromethoxy or 2,2-difluoroethoxy, and R3 and R31 are both hydrogen.

Another special embodiment of the compounds of the present invention include those compounds of formula I in which R1 and R2 are independently 1-2C-alkoxy, 2,2-difluoroethoxy, or completely or predominantly fluorine-substituted 1-2C-alkoxy, and R3, R31 and R6 are all hydrogen.

Another special embodiment of the compounds of the present invention include those compounds of formula I in which one of R1 and R2 is methoxy, and the other is methoxy, ethoxy, difluoromethoxy or 2,2-difluoroethoxy, and R3, R31 and R6 are both hydrogen.

Another special embodiment of the compounds of the present invention include those compounds of formula I in which R1 is ethoxy or, particularly, methoxy, and R2 is methoxy, or, particularly, ethoxy, difluoromethoxy or 2,2-difluoroethoxy, and R3 and R31 are both hydrogen.

Another special embodiment of the compounds of the present invention include those compounds of formula I in which R1 is methoxy, and R2 is methoxy, ethoxy, difluoromethoxy or 2,2-difluoroethoxy, and R3 and R31 are both hydrogen.

Another special embodiment of the compounds of the present invention include those compounds of formula I in which R1 is methoxy, and R2 is ethoxy, difluoromethoxy or 2,2-difluoroethoxy, and R3 and R31 are both hydrogen.

Another special embodiment of the compounds of the present invention include those compounds of formula I in which one of R1 and R2 is 2,2-difluoroethoxy, and the other is different from 2,2-difluoroethoxy, and R3 and R31 are both hydrogen.

Another special embodiment of the compounds of the present invention include those compounds of formula I in which R1 is ethoxy or, particularly, methoxy, and R2 is 2,2-difluoroethoxy, and R3 and R31 are both hydrogen.

Another special embodiment of the compounds of the present invention include those compounds of formula I in which R1 is methoxy, and R2 is 2,2-difluoroethoxy, and R3 and R31 are both hydrogen.

Another special embodiment of the compounds of the present invention include those compounds of formula I in which R1 is methoxy, and R2 is ethoxy, and R3 and R31 are both hydrogen.

Another special embodiment of the compounds of the present invention include those compounds of formula I in which R1 is methoxy, and R2 is difluoromethoxy, and R3 and R31 are both hydrogen.

Another special embodiment of the compounds of the present invention include those compounds of formula I, in which R5 or, particularly, R4 is the radical (1-4C-alkylcarbonyl)—O— such as e.g. acetoxy, or hydroxyl, and all the other substituents are as defined in any compound which is said to be mentioned above.

Another special embodiment of the compounds of the present invention include those compounds of formula I in which R5 or, particularly, R4 is hydroxyl.

Another special embodiment of the compounds of the present invention include those compounds of formula I according to aspect 1.

Another special embodiment of the compounds of the present invention include those compounds of formula I according to aspect 2.

Another special embodiment of the compounds of the present invention include those compounds of formula I in which R6 is hydrogen.

Another special embodiment of the compounds of the present invention include those compounds of formula I according to aspect 1 in which R9 is pyridinyl substituted by R91 and R92.

Another special embodiment of the compounds of the present invention include those compounds of formula I according to aspect 1 in which R8 is hydrogen or 1-4C-alkyl, and R9 is 1-4C-alkyl, cyclopropyl or cyclobutyl.

Another special embodiment of the compounds of the present invention include those compounds of formula I according to aspect 1, in which R8 is isopropyl and R9 is isopropyl.

Another special embodiment of the compounds of the present invention include those compounds of formula I according to aspect 1 in which R8 is hydrogen and R9 is cyclopropyl or cyclobutyl.

Another special embodiment of the compounds of the present invention include those compounds of formula I according to aspect 1, in which R8 is isopropyl and R9 is isopropyl.

A preferred embodiment according to the present invention is embodiment a.

A further preferred embodiment of the compounds of the present invention include compounds according to embodiment a, in which R5 and R41 are both hydrogen, and in which R1 and R2 are independently 1-2C-alkoxy, 2,2-difluoroethoxy, or completely or predominantly fluorine-substituted 1-2C-alkoxy, and R3, R31 and R6 are all hydrogen.

A yet further preferred embodiment of the compounds of the present invention include compounds according to embodiment a, in which R5 is hydrogen, and in which R1 is methoxy, and R2 is ethoxy, difluoromethoxy or 2,2-difluoroethoxy, and R3 and R31 are both hydrogen.

A still yet further preferred embodiment of the compounds of the present invention include compounds according to embodiment a, in which R6, R5 and R41 are all hydrogen, and in which R1 is methoxy, and R2 is ethoxy, difluoromethoxy or 2,2-difluoroethoxy, and R3 and R31 are both hydrogen.

Suitable compounds according to the present invention more worthy to be mentioned include those compounds of formula I, in which R5 or, particularly, R4 is hydroxyl.

Exemplary compounds according to the present invention may include, without being restricted thereto, compounds selected from the group consisting of
4-((2RS,4aRS,10bRS)-2-Hydroxy-8,9-dimethoxy-1,2,3,4,4a,10b-hexahydro-phenanthridin-6-yl)-N-(2-morpholin4-yl-ethyl)-benzamide
4-((2RS,4aRS,10bRS)-2-Hydroxy-8,9-dimethoxy-1,2,3,4,4a,10b-hexahydro-phenanthridin-6-yl)-N-(3-morpholin-4-yl-propyl)-benzamide
4-((2RS,4aRS,10bRS)-2-Hydroxy-8,9-dimethoxy-1,2,3,4,4a,10b-hexahydro-phenanthridin-6-yl)-N-(4-methyl-piperazin-1-yl)-benzamide
4-((2RS,4aRS,10bRS)-2-Hydroxy-8,9-dimethoxy-1,2,3,4,4a,10b-hexahydro-phenanthridin-6-yl)-N-morpholin-4-ylbenzamide
({1-[4-((2RS,4aRS,10bRS)-2-Hydroxy-8,9-dimethoxy-1,2,3,4,4a,10b-hexahydro-phenanthridin-6-yl)-phenyl]-methanoyl}-methyl-amino)-acetic acid methyl ester
4-((2RS,4aRS,10bRS)-2-Hydroxy-8,9-dimethoxy-1,2,3,4,4a,10b-hexahydro-phenanthridin-6-yl)-N-quinolin-3-yl-benzamide
4-((2RS,4aRS,10bRS)-2-Hydroxy-8,9-dimethoxy-1,2,3,4,4a,10b-hexahydro-phenanthridin-6-yl)-N-(2-pyridin-2-yl-ethyl)-benzamide
1-[4-((2RS,4aRS,10bRS)-2-Hydroxy-8,9-dimethoxy-1,2,3,4,4a,10b-hexahydro-phenanthridin-6-yl)-phenyl]-1-(4-pyridin-2-yl-piperazin-1-yl)-methanone
1-[4-((2RS,4aRS,10bRS)-2-Hydroxy-8,9-dimethoxy-1,2,3,4,4a,10b-hexahydro-phenanthridin-6-yl)-phenyl]-1-[4-(2-morpholin-4-yl-ethyl)-piperazin-1-yl]-methanone
N-Ethyl-4-((2RS,4aRS,10bRS)-2-hydroxy-8,9-dimethoxy-1,2,3,4,4a,10b-hexahydro-phenanthridin-6-yl)-N-(2-methoxy-ethyl)-benzamide
N-Cyclopropyl-4-((2RS,4aRS,10bRS)-2-hydroxy8,9-dimethoxy-1,2,3,4,4a,10b-hexahydro-phenanthridin-6-yl)-benzamide
2-(4-{1-[4-((2RS,4aRS,10bRS)-2-Hydroxy-8,9-dimethoxy-1,2,3,4,4a,10b-hexahydro-phenanthridin-6-yl)-phenyl]-methanoyl}-piperazin-1-yl)-1-pyrrolidin-1-yl-ethanone
2-(4-{1-[4-((2RS,4aRS,10bRS)-2-Hydroxy-8,9-dimethoxy-1,2,3,4,4a,10b-hexahydro-phenanthridin-6-yl)-phenyl]-methanoyl}-piperazin-1-yl)-N-pyridin-3-yl-acetamide
4-((2RS,4aRS,10bRS)-2-Hydroxy-8,9-dimethoxy-1,2,3,4,4a,10b-hexahydro-phenanthridin-6-yl)-N,N-dimethyl-benzamide
2-(4-{1-[4-((2RS,4aRS,10bRS)-2-Hydroxy-8,9-dimethoxy-1,2,3,4,4a,10b-hexahydro-phenanthridin-6-yl)-phenyl]-methanoyl}-piperazin-1-yl)-N-pyridin-2-yl-acetamide
2-(4-{1-[4-((2R,4aR,10bR)-2-Hydroxy-8,9-dimethoxy-1,2,3,4,4a,10b-hexahydro-phenanthridin-6-yl)-phenyl]-methanoyl}-piperazin-1-yl)-N,N-dimethyl-acetamide
2-(4-{1-[4-((2RS,4aRS,10bRS)-2-Hydroxy-8,9-dimethoxy-1,2,3,4,4a,10b-hexahydro-phenanthridin-6-yl)-phenyl]-methanoyl}-piperazin-1-yl)-1-morpholin-4-yl-ethanone
1-[4-((2RS,4aRS,10bRS)-2-Hydroxy-8,9-dimethoxy-1,2,3,4,4a,10b-hexahydro-phenanthridin-6-yl)-phenyl]-1-(4-pyridin-4-yl-piperazin-1-yl)-methanone
1-[4-((2RS,4aRS,10bRS)-2-Hydroxy-8,9-dimethoxy-1,2,3,4,4a,10b-hexahydro-phenanthridin-6-yl)-phenyl]-1-morpholin-4-yl-methanone
4-((2RS,4aRS,10bRS)-2-Hydroxy-8,9-dimethoxy-1,2,3,4,4a,10b-hexahydro-phenanthridin-6-yl)-N-(2-pyridin-4-yl-ethyl)-benzamide
4-((2RS,4aRS,10bRS)-2-Hydroxy-8,9-dimethoxy-1,2,3,4,4a,10b-hexahydro-phenanthridin-6-yl)-N-(2-pyridin-3-yl-ethyl)-benzamide
4-((2RS,4aRS,10bRS)-2-Hydroxy-8,9-dimethoxy-1,2,3,4,4a,10b-hexahydro-phenanthridin-6-yl)-benzoic acid N′-(1-morpholin-4-yl-methanoyl)-hydrazide
N-(2,6-Dimethoxy-pyridin-3-yl)-4-((2RS,4aRS,10bRS)-2-hydroxy-8,9-dimethoxy-1,2,3,4,4a,10b-hexahydro-phenanthridin-6-yl)-benzamide
4-[(2RS,4aRS,10bRS)-9-(1,1-Difluoro-methoxy)-2-hydroxy-8-methoxy-1,2,3,4,4a,10b-hexahydro-phenanthridin-6-yl]-N,N-dimethyl-benzamide
N-Cyclopropyl-4-[(2RS,4aRS,10bRS)-9-(1,1-difluoro-methoxy)-2-hydroxy-8-methoxy-1,2,3,4,4a,10b-hexahydro-phenanthridin-6-yl-benzamide
4-[(2RS,4aRS,10bRS)-9-(1,1-Difluoro-methoxy)-2-hydroxy-8-methoxy-1,2,3,4,4a,10b-hexahydro-phenanthridin-6-yl]-N,N-bis-(2-methoxy-ethyl)-benzamide
4-[(2RS,4aRS,10bRS)-9-(1,1-Difluoro-methoxy)-2-hydroxy-8-methoxy-1,2,3,4,4a,10b-hexahydro-phenanthridin-6-yl]-N-(2-morpholin-4-yl-ethyl)-benzamide
4-[(2RS,4aRS,10bRS)-9-(1,1-Difluoro-methoxy)-2-hydroxy-8-methoxy-1,2,3,4,4a,10b-hexahydro-phenanthridin-6-yl]-N-(3-morpholin4-yl-propyl)-benzamide
1-{4-[(2RS,4aRS,10bRS)-9-(1,1-Difluoro-methoxy)-2-hydroxy-8-methoxy-1,2,3,4,4a,10b-hexahydro-phenanthridin-6-yl]-phenyl}-1-[4-(2-morpholin-4-yl-ethyl)-piperazin-1-yl]-methanone
1-{4-[(2RS,4aRS,10bRS)-9-(1,1-Difluoro-methoxy)-2-hydroxy-8-methoxy-1,2,3,4,4a,10b-hexahydro-phenanthridin-6-yl]-phenyl}-1-(4-pyridin-4-yl-piperazin-1-yl)-methanone
2-[4-(1-{4-[(2RS,4aRS,10bRS)-9-(1,1-Difluoro-methoxy)-2-hydroxy-8-methoxy-1,2,3,4,4a,10b-hexahydro-phenanthridin-6-yl]-phenyl}-methanoyl)-piperazin-1-yl]-N-pyridin-2-yl-acetamide
2-[4-(1-{4-[(2RS,4aRS,10bRS)-9-(1,1-Difluoro-methoxy)-2-hydroxy-4-methoxy-1,2,3,4,4a,10b-hexahydro-phenanthridin-6-yl]-phenyl}-methanoyl)-piperazin-1-yl]-1-morpholin-4-yl]ethanone
1-{4-[(2RS,4aRS,10bRS)-9-(1,1-Difluoro-methoxy)-2-hydroxy-8-methoxy-1,2,3,4,4a,10b-hexahydro-phenanthridin-6-yl]-phenyl}-1-pyrrolidin-1-yl-methanone
2-[4-(1-{4-[(2RS,4aRS,10bRS)-9-(1,1-Difluoro-methoxy)-2-hydroxy4-methoxy-1,2,3,4,4a,10b-hexahydro-phenanthridin-6-yl]-phenyl}-methanoyl)-piperazin-1-yl]-N,N-dimethyl-acetamide
1-{4-[(2RS,4aRS,10bRS)-9-(1,1-Difluoro-methoxy)-2-hydroxy-8-methoxy-1,2,3,4,4a,10b-hexahydro-phenanthridin-6-yl]-phenyl}-1-[4-(2-dimethylamino-ethyl)-piperazin-1-yl]-methanone
N-(2,6-Dimethoxy-pyridin-3-yl)-4-((2R,4aR,10bR)-2-hydroxy-8,9-dimethoxy-1,2,3,4,4a,10b-hexahydro-phenanthridin-6-yl)-benzamide
N-(2,6-Dimethoxy-pyridin-3-yl)-4-((2S,4aS,10bS)-2-hydroxy-8,9-dimethoxy-1,2,3,4,4a,10b-hexahydro-phenanthridin-6-yl)-benzamide
N-Cyclopropyl-4-[(2R,4aR,10bR)-9-(1,1-difluoro-methoxy)-2-hydroxy-8-methoxy-1,2,3,4,4a,10b-hexahydro-phenanthridin-6-yl]-benzamide
N-Cyclopropyl-4-[(2S,4aS,10bS)-9-(1,1-difluoro-methoxy)-2-hydroxy-8-methoxy-1,2,3,4,4a,10b-hexahydro-phenanthridin-6-yl]-benzamide
N-Cyclopropyl-4-((2R,4aR,10bR)-9-ethoxy-2-hydroxy-8-methoxy-1,2,3,4,4a,10b-hexahydro-phenanthridin-6-yl)-benzamide
N-Cyclobutyl-4-((2R,4aR,10bR)-9-ethoxy-2-hydroxy-8-methoxy-1,2,3,4,4a,10b-hexahydro-phenanthridin-6-yl)-benzamide
4-((2R,4aR,10bR)-9-Ethoxy-2-hydroxy-8-methoxy-1,2,3,4,4a,10b-hexahydro-phenanthridin-6-yl)-N,N-diisopropyl-benzamide
N-Cyclopropyl-3-((2R,4aR,10bR)-9-ethoxy-2-hydroxy-8-methoxy-1,2,3,4,4a,10b-hexahydro-phenanthridin-6-yl)-benzamide
N-Cyclobutyl-3-((2R,4aR,10bR)-9-ethoxy-2-hydroxy-8-methoxy-1,2,3,4,4a,10b-hexahydro-phenanthridin-6-yl)-benzamide
3-((2R,4aR,10bR)-9-Ethoxy-2-hydroxy-8-methoxy-1,2,3,4,4a,10b-hexahydro-phenanthridin-6-yl)-N,N-diisopropyl-benzamide and
N-Cyclopropyl-4-((3S,4aR,10bR)-9-ethoxy-3-hydroxy-8-methoxy-1,2,3,4,4a,10b-hexahydro-phenanthridin-6-yl)-benzamide,
   the enantiomers, as well as the salts, the N-oxides and the salts of the N-oxides of these compounds and enantiomers.

Preferably, any or all of those compounds of formula I according to embodiment a, in which R3, R31, R41 and R5 are all hydrogen, which are described by way of example as final compounds in the following examples and, particularly, the enantiomers thereof, particularly those having the formula Ia*****, as well as the salts of these compounds and enantiomers, are to be mentioned as a particular interesting aspect of the present invention.

The compounds of formula I are chiral compounds having chiral centers at least in positions 4a and 10b and depending on the meanings of R3, R31, R4 and R5 additional chiral centers in positions 1, 2, 3 and 4.

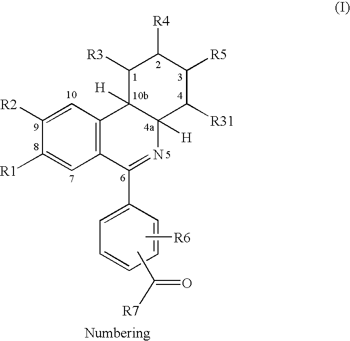

The invention includes all conceivable stereoisomers in pure form as well as in any mixing ratio. Preference is given to compounds of formula I in which the hydrogen atoms in positions 4a and 10b are in the cis position relative to one another. The pure cis enantiomers and their mixtures in any mixing ratio and including the racemates are more preferred in this context.

Particularly preferred in this context are those compounds of formula 1, which have with respect to the positions 4a and 10b the configuration shown in formula (I*):

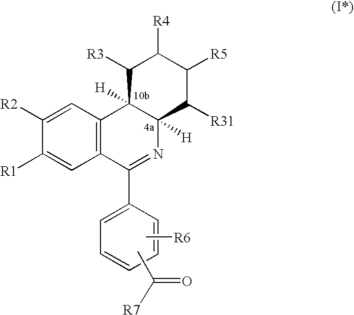

If, for example, in compounds of formula I* R3, R31 and R5 have the meaning hydrogen and R4 has the meaning —OR41, then the configuration—according to the rules of Cahn, Ingold and Prelog—is R in the 4a position and R in the 10b position.

Further preferred compounds of the formula I according to embodiment a are those which have, with respect to the positions 2, 4a and 10b, the same configuration as shown in the formulae Ia** and Ia*** and Ia****:

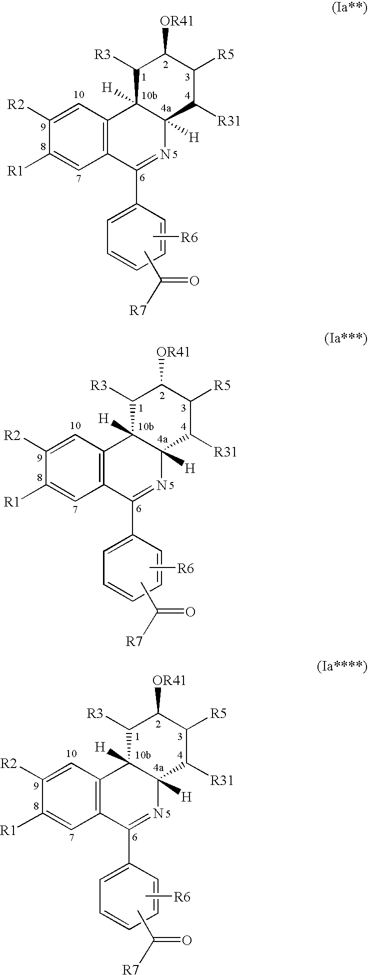

If, for example in compounds of the formula Ia** R3, R31 and R5 have the meaning hydrogen, then the configuration—according the rules of Cahn, Ingold and Prelog—is S in the position 2, R in the position 4a and R in the position 10b.

If, for example in compounds of the formula Ia*** R3, R31 and R5 have the meaning hydrogen, then the configuration—according the rules of Cahn, Ingold and Prelog—is R in the position 2, S in the position 4a and S in the position 10b.

If, for example in compounds of the formula Ia**** R3, R31 and R5 have the meaning hydrogen, then the configuration—according the rules of Cahn, Ingold and Prelog—is S in the position 2, S in the position 4a and S in the position 10b.

In more particular preferred compounds of the formula I according embodiment a are those which have, with respect to the positions 2, 4a and 10b, the same configuration as shown in the formula Ia*****:

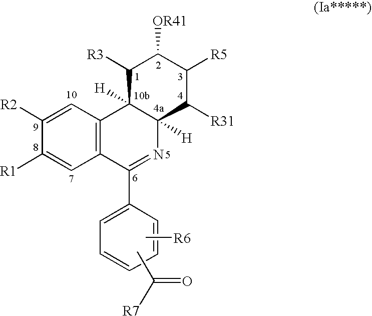

If, for example in compounds of the formula Ia***** R3, R31 and R5 have the meaning hydrogen, then the configuration—according the rules of Cahn, Ingold and Prelog—is R in the position 2, R in the position 4a and R in the position 10b.

Preferred compounds of the formula I according to embodiment b are those which have, with respect to the positions 3, 4a and 10b, the same configuration as shown in the formulae Ib** and Ib*** and Ib****:

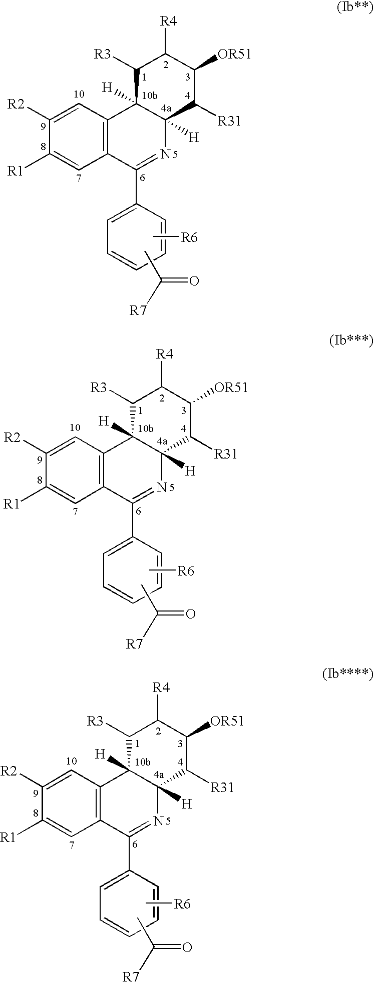

If, for example in compounds of the formula Ib** R3, R31 and R5 have the meaning hydrogen, then the configuration—according the rules of Cahn, Ingold and Prelog—is R in the position 3, R in the position 4a and R in the position 10b.

If, for example in compounds of the formula Ib*** R3, R31 and R5 have the meaning hydrogen, then the configuration—according the rules of Cahn, Ingold and Prelog—is S in the position 3, S in the position 4a and S in the position 10b.

If, for example in compounds of the formula Ib**** R3, R31 and R5 have the meaning hydrogen, then the configuration—according the rules of Cahn, Ingold and Prelog—is R in the position 3, S in the position 4a and S in the position 10b.

More preferred compounds of the formula I according to embodiment b are those which have, with respect to the positions 3, 4a and 10b, the same configuration as shown in the formula Ib*****:

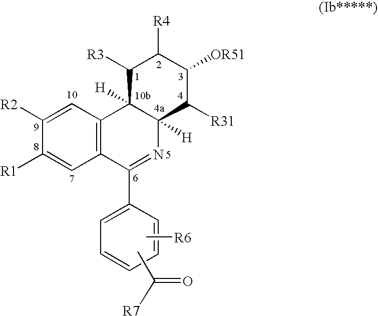

If, for example in compounds of the formula Ib***** R3, R31 and R5 have the meaning hydrogen, then the configuration—according the rules of Cahn, Ingold and Prelog—is S in the position 3, R in the position 4a and R in the position 10b.

Within the meaning of the embodiments a and b according to this invention, compounds of formula Ia***** are in particular to be emphasized.

The enantiomers can be separated in a manner known per se (for example by preparation and separation of appropriate diastereoisomeric compounds). Thus, e.g. an enantiomer separation can be carried out at the stage of the starting compounds having a free amino group such as starting compounds of formulae VIIa or Xb as defined below.

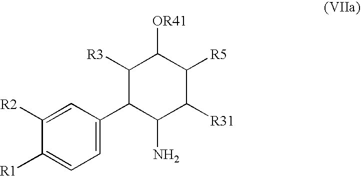

Separation of the enantiomers can be carried out, for example, by means of salt formation of the racemic compounds of the formulae VIIa or Xb with optically active acids, preferably carboxylic acids, subsequent resolution of the salts and release of the desired compound from the salt. Examples of optically active carboxylic acids which may be mentioned in this connection(n are the enantiomeric forms of mandelic acid, tartaric acid, O,O′-dibenzoyltartaric acid, camphoric acid, quinic acid, glutamic acid, pyroglutamic acid, malic acid, camphorsulfonic acid, 3-bromocamphorsulfonic acid, α-methoxyphenylacetic acid, α-methoxy-α-trifluoromethylphenylacetic acid and 2-phenyl propionic acid. Alternatively, enantiomerically pure starting compounds can be prepared via asymmetric syntheses. Enantiomerically pure starting compounds as well as enantiomerically pure compounds of the formula I can be also obtained by chromatographic separation on chiral separating columns; by derivatization with chiral auxiliary reagents, subsequent diastereomer separation and removal of the chiral auxiliary group; or by (fractional) crystallization from a suitable solvent.

The compounds according to the invention can be prepared, for example, as shown in the reaction schemes below and according to the following specified reaction steps, or, particularly, in a manner as described by way of example in the following examples, or analogously or similarly thereto according to preparation procedures or synthesis strategies known to the person skilled in the art.

Compounds of formula I, in which R1, R2, R3, R31, R4, R5, R6 and R7 have the meanings mentioned above, can be obtained as outlined in reaction scheme 1 and as described as follows starting with compounds of formula IV, in which C(O)OR stands for a suitable ester group such as an alkyl ester (preferably a methyl ester group).

On the one hand, compounds of formula I may be obtained from the compounds of formula IV by direct reaction with compounds of formula R7-H, in which R7 has the meanings given above.

On the other hand the compounds of formula IV can be first saponified to give the benzoic acid derivatives of formula III which can then be amidified with compounds of formula R7-H in a manner customary per se to the skilled person.

Compounds of formula III, in which R4 or R5 is hydroxyl, (obtainable, for example, from corresponding compounds of formula IV, in which R4 or R5 is acyloxy, by the abovementioned saponification step affording, beside the free benzoic acid group, the respective desacylated free hydroxyl group) should be protected by a suitable temporary protective group or, preferably, via acylation, such as e.g. via acetylation, reaction known per se to the skilled person or as described in the following examples, using e.g. the acid chlorides, before further reaction.

Benzoic acid derivatives of formula III can then be activated prior to the amide bond forming reaction with compounds of formula R7-H, for example by forming an acid halide or acid anhydride, (compounds of formula 2, in which Y is a suitable leaving group), or by using coupling agents known to the person skilled in the art, such as, for example, N,N′-dicyclohexylcarbodiimide, N′-(3-dimethylaminopropyl)-N-ethylcarbodiimide hydrochloride (EDCl) or 2-(1H-benzotriazole-1-yl)-1,1,3,3-tetramethyluronium hexafluorophosphate (HBTU).

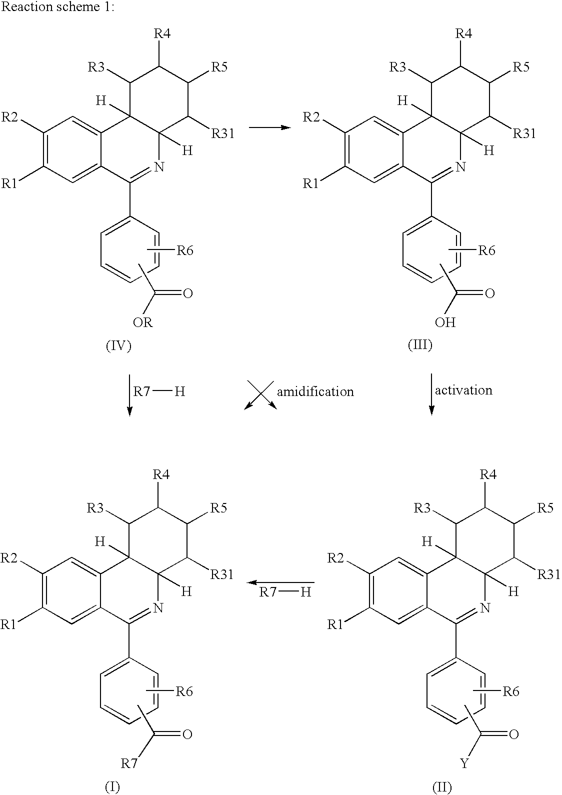

Optionally, compounds of the formula I can be also converted into further compounds of the formula I by methods known to one of ordinary skill in the art. More specifically, for example, from compounds of the formula I in which
a) R41 or R51 is hydrogen, the corresponding ester compounds can be obtained by esterification reactions;
b) R41 or R51 is hydrogen, the corresponding ether compounds can be obtained by etherification reactions;
c) R41 or R51 is an acyl group, such as e.g. acetyl, the corresponding hydroxyl compounds can be obtained by deesterification (e.g. saponification) reactions;

The methods mentioned under a), b) and c) are expediently carried out analogously to the methods known to the person skilled in the art or as described by way of example in the following examples.

Optionally, compounds of the formula I can be converted into their salts, or, optionally, salts of the compounds of the formula I can be converted into the free compounds.

In addition, the compounds of the formula I can be converted, optionally, into their N-oxides, for example with the aid of hydrogen peroxide in methanol or with the aid of m-chloroperoxybenzoic acid in dichloromethane. The person skilled in the art is familiar on the basis of his/her expert knowledge with the reaction conditions which are specifically necessary for carrying out the N-oxidation.

Compounds of formula IV according to embodiment a or b (i.e. compounds of formulae IVa or IVb, respectively) can be obtained as described as follows.

In the first reaction step of the synthesis route shown in scheme 2, compounds of the formula VIIIa, in which R1, R2, R3, R31, R41 and R5 have the meanings mentioned above in embodiment a whereby R41 is other than hydrogen, are prepared from the corresponding compounds of the formula IXa by introduction of the group R41. The introduction reaction is carried out in a manner habitual per se for an etherification or esterification reaction or as described by way of example in the following examples.

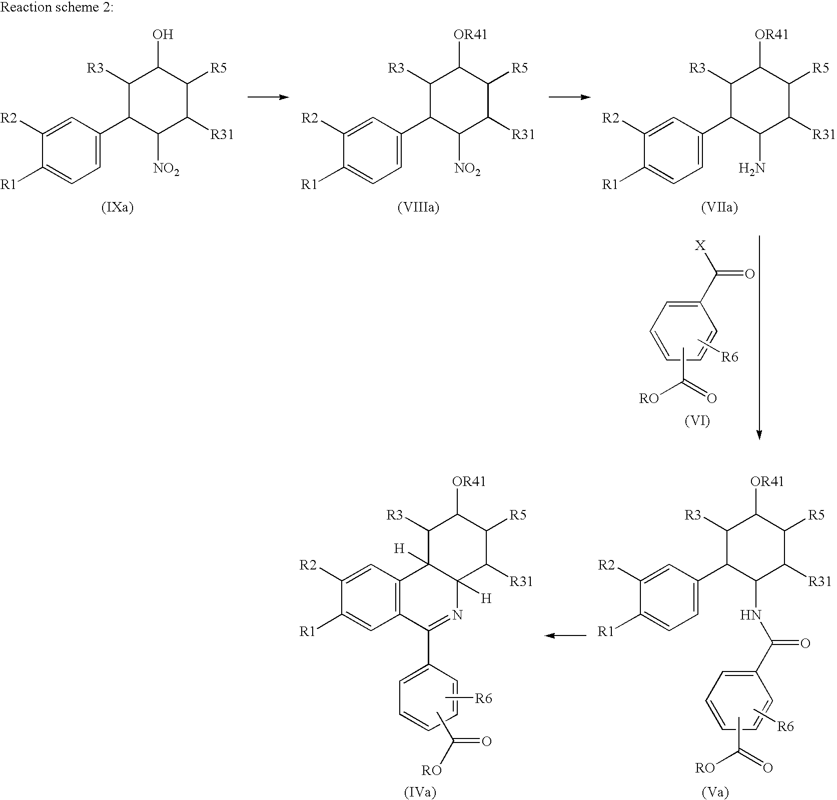

In the next reaction step of the synthesis route shown, the nitro group of compounds of the formula VIIIa, in which R1, R2, R3, R31, R41 and R5 have the meanings mentioned above in embodiment a whereby R41 is other than hydrogen, is reduced to the amino group of the corresponding compounds of the formula VIIa. Said reduction is carried out in a manner known to the person skilled in the art, for example as described in J. Org. Chem. 1962, 27, 4426 or as described in the following examples. In more detail, the reduction can be carried out, for example, by catalytic hydrogenation, e.g. in the presence of Raney nickel or a noble metal catalyst such as palladium on active carbon, in a suitable solvent such as methanol or ethanol at room temperature and under normal or elevated pressure. Optionally, a catalytic amount of an acid, such as, for example, hydrochloric acid, can be added to the solvent. Preferably, however, the reduction is carried out using a hydrogen-producing mixture, for example, metals such as zinc, zinc-copper couple or iron with organic acids such as acetic acid or mineral acids such as hydrochloric acid. More preferably, the reduction is carried out using a zinc-copper couple in the presence of an organic or an inorganic acid. Such a zinc-copper couple is accessible in a way known to the person of ordinary skill in the art.

Compounds of the formula Va, in which R1, R2, R3, R31, R41, R5 and R6 have the meanings indicated above in embodiment a whereby R41 is other than hydrogen and C(O)OR stands for a suitable ester group, preferably the methyl ester group, are accessible from the corresponding compounds of the formula VIIa, by reaction with corresponding compounds of the formula VI, in which X represents a suitable leaving group, preferably a chlorine atom.

Alternatively, compounds of the formula Va can also be prepared from the corresponding compounds of the formula VIIa and corresponding compounds of the formula VI, in which X is hydroxyl, by reaction with amide bond linking reagents known to the person skilled in the art. Exemplary amide bond linking reagents known to the person skilled in the art which may be mentioned are, for example, the carbodiimides (e.g. dicyclohexylcarbodiimide or, preferably, 1-ethyl-3-(3-dimethylaminopropyl)carbodiimide hydrochloride), azodicarboxylic acid derivatives (e.g. diethyl azodicarboxylate), uronium salts [e.g. O(benzotriazol-1-yl)-N,N,N′,N′-tetramethyluronium tetrafluo roborate or O-(benzotriazol-1-yl)-N,N,N′,N′-tetramthyluronium-hexafluorophosphate] and N,N′-carbonyldiimidazole. In the scope of this invention preferred amide bond linking reagents are uronium salts and, particularly, carbadiimides, preferably, 1-ethyl-3-(3-dimethylaminopropyl) carbodiimide hydrochloride.

Compounds of the formula VI are either known or can be prepared in a known manner.

Compounds of the formula IVa, in which R1, R2, R3, R31, R41, R5 and R6 and have the meanings as given in embodiment a whereby R41 is other than hydrogen and C(O)OR stands for a suitable ester group, preferably the methyl ester group, can be obtained by cyclocondensation of corresponding compounds of the formula Va.

Said cyclocondensation reaction is carried out in a manner known per se to the person skilled in the art or as described by way of example in the following examples, according to Bischler-Napieralski (e.g. as described in J. Chem. Soc., 1956, 4280-4282) in the presence of a suitable condensing agent, such as, for example, polyphosphoric acid, phosphorus pentachloride, phosphorus pentoxide or phosphorus oxychloride, in a suitable inert solvent, e.g. in a chlorinated hydrocarbon such as chloroform, or in a cyclic hydrocarbon such as toluene or xylene, or another inert solvent such as isopropyl acetate or acetonitrile, or without further solvent using an excess of condensing agent, at reduced temperature, or at room temperature, or at elevated temperature or at the boiling temperature of the solvent or condensing agent used. If necessary, said cyclocondensation reaction can be carried out in the presence of one or more suitable Lewis Acids such as, for example, suitable metal halogenides (e.g. chlorides) or sulphonates (e.g. triflates), including rare earth metal salts, such as e.g. anhydrous aluminum trichloride, aluminum tribromide, zinc chloride, boron trifluoride ethereate, titanium tetrachloride or, in particular, tin tetrachloride, and the like.

Below reaction scheme 3 shows the synthesis of compounds of the formula IXa, in which R1, R2, R3, R31 and R5 have the meanings indicated above in embodiment a, from corresponding compounds of the formula Xa via reduction reaction of the carbonyl group. Suitable reducing agents for the above mentioned reduction reaction may include, for example, metal hydride compounds such as, for example, diisopropylaluminium hydride, borane, sodium borohydride, sodium triacetoxyborohydride, sodium cyanoborohydride, zinc borohydride, potassium tri-sec-butylborohydride, sodium tri-sec-butylborohydride, lithium tri-sec-butylborohydride, β-isopinocampheyl-9-borabicyclo[3.3.1]nonane and the like. The preferred examples of said reducing agents are sodium cyanoborohydride, β-isopinocampheyl-9-borabicyclo[3.3.1]nonane and potassium tri-sec-butylborohydride. The most preferred examples of the abovementioned reducing agents are β-isopinocampheyl-9-borabicyclo[3.3.1]nonane and potassium tri-sec-butylborohydride, which both allow to prepare compounds of the formula IXa stereoselectively. “Stereoselectively” in this connection means that those compounds of the formula IXa, in which the hydrogen atoms in positions 1 and 3 are located at the opposite side of the plane defined by the cyclohexane ring, are obtained preferentially.

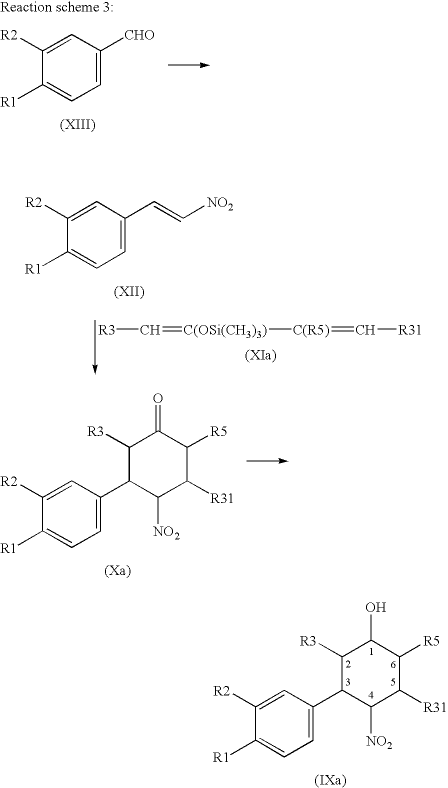

The compounds of the formula Xa, in which R1, R2, R3, R31 and R5 have the meanings mentioned in embodiment a, are either known or can be obtained by the reaction of compounds of the formula XII, in which R1 and R2 have the meanings mentioned above, with compounds of the formula XIa, in which R3, R31 and R5 have the meanings mentioned above in embodiment a. The cycloaddition reaction is carried out in a manner known to the person skilled in the art according to Diels-Alder, e.g. as described in J. Amer. Chem. Soc. 1957, 79, 6559 or in J. Org. Chem. 1952, 17, 581 or as described in the following examples.

Compounds of the formulae IXa or VIIIa, in which the phenyl ring and the nitro group are trans to one another, can be converted in a manner known to the person skilled in the art into the corresponding cis compounds, e.g. as described in J. Amer. Chem. Soc. 1957, 79, 6559 or as described in the following examples.

The compounds of the formulae XIa and XII are either known or can be prepared in a known manner. The compounds of the formula XII can be prepared, for example, in a manner known to the person skilled in the art from corresponding compounds of the formula XII as described, for example, in J. Chem. Soc. 1951, 2524 or in J. Org. Chem. 1944, 9, 170 or as described in the following examples.

The compounds of the formula XIII, in which R1 and R2 have the meanings indicated above, are either known or can be prepared in a manner known to the person skilled in the art, as described, for example, in Ber. Dtsch. Chem. Ges. 1925, 58, 203.

Compounds of formula IVb according to embodiment b can be prepared as described and shown in reaction scheme 4 below.

In the first reaction step in reaction scheme 4 below, the nitro group of compounds of the formula XIb, in which R1, R2, R3, R31 and R4 have the meanings indicated in embodiment b above, is reduced to obtain corresponding compounds of the formula Xb. Said reduction reaction is carried out in a manner known to the person skilled in the art, for example as described in J. Org. Chem. 1962, 27, 4426 or as described in the following examples. More specifically, the reduction can be carried out, for example, by contacting compounds of the formula XIb with a hydrogen-producing mixture such as, preferably, metallic zinc in a mildly acidic medium such as acetic acid in a lower alcohol such as methanol or ethanol at room temperature or at elevated temperature or, preferably, at the boiling temperature of the solvent mixture. Alternatively, the reduction can be carried out by selective reduction of the nitro group in a manner known to the person skilled in the art, for example by hydrogen transfer reaction in the presence of a metal catalyst, for example palladium or preferably Raney nickel, in a suitable solvent, preferably a lower alcohol, using, for example ammonium formiate or preferably hydrazine hydrate as hydrogen donor.

Compounds of the formula Xb obtained can be reacted, for example, as described by way of example in the following examples with compounds of the formula VI, in which R6 has the meanings given above, C(O)OR stands for a suitable ester group, preferably the methyl ester group, and X represents a suitable leaving group, preferably a chlorine atom, to give corresponding compounds of the formula IXb.

Alternatively, compounds of the formula IXb, in which R1, R2, R3, R31, R4 and R6 have the meanings given above in embodiment b and C(O)OR stands for said suitable ester group, can also be prepared, for example, from corresponding compounds of the formula Xb and corresponding compounds of the formula VI, in which X is hydroxyl, by reaction with amide bond linking reagents known to the person skilled in the art. Exemplary amide bond linking reagents known to the person skilled in the art which may be mentioned are, for example, the carbodiimides (e.g. dicyclohexylcarbodiimide or, preferably, 1-ethyl-3-(3-dimethylaminopropyl)carbodiimide hydrochloride), azodicarboxylic acid derivatives (e.g. diethyl azodicarboxylate), uronium salts [e.g. O-(benzotriazol-1-yl)-N,N,N′,N′-tetramethyluronium tetrafluoroborate or O-(benzotriazol-1-yl)-N,N,N′,N′-tetramthyl-uronium-hexafluorophosphate] and N,N′-carbonyidiimidazole. In the scope of this invention preferred amide bond linking reagents are uronium salts and, particularly, carbodiimides, preferably, 1-ethyl-3-(3-dimethylaminopropyl)carbodiimide hydrochloride.

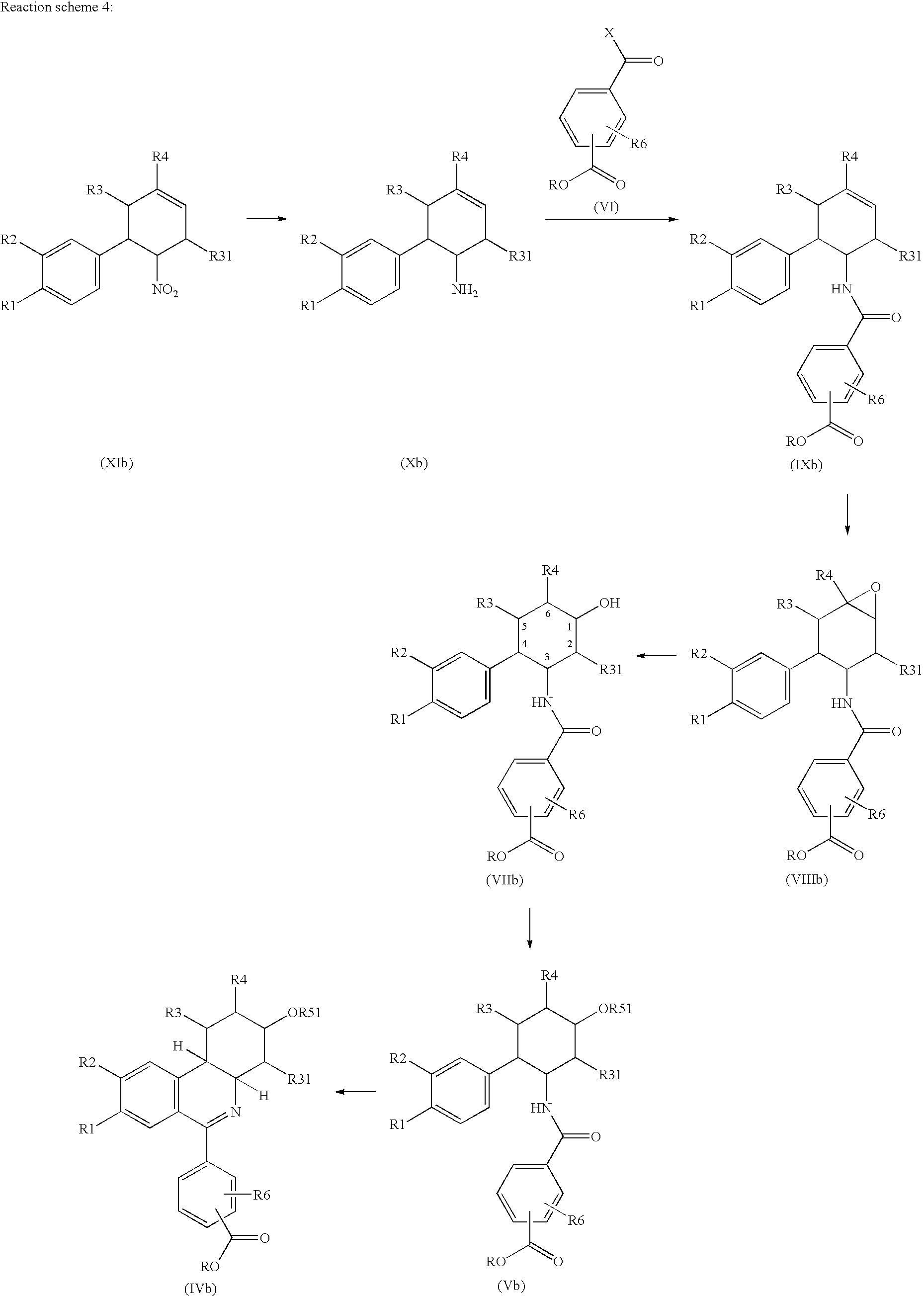

In the next step compounds of the formula IXb can be converted into corresponding compounds of the formula VIIIb by epoxidation reaction, which can be carried out as described in the following examples or in a manner known to one of ordinary skill in the art employing, for example, suitable epoxidation methods or suitable epoxidation reagents such as, for example, peracids (e.g. m-chloroperbenzoic acid) or organic or inorganic peroxides (e. g. dimethyidioxirane, hydrogene peroxide or persulfates).

Compounds of the formula VIIIb obtained can be reduced by art-known methods to corresponding compounds of the formula VIIb. More specifically, said reduction reaction can be performed employing, for example, as described by way of example in the following examples sodium borohydride as reductant.

Alternatively, said reduction reaction can be also carried out using, for example, lithium aluminium hydride or a reductive mixture comprising noble metals, such as platinium dioxide or palladium, and a suitable hydrogen donor. With the aid of each of those said reduction methods, compounds of the formula VIIIb can be converted largely regio- and diastereoselectively into compounds of the formula VIIb, wherein the hydroxyl radical in position 1 and the amido radical in position 3 are located at the same side of the plane defined by the cyclohexane ring.

It is moreover known to one of ordinary skill of the art, that the absolute configuration of a chiral carbon atom, preferably, to which a hydroxyl group and a hydrogen atom are bonded, can be inverted. Thus the configuration of the carbon atom in position 1 of compounds of the formula VIIb can be optionally inverted. Said inversion of configuration of position 1 of compounds of the formula VIIb can be achieved in a manner familiar to the person skilled in the art, for example by derivatization of position 1 with a suitable leaving group and subsequent replacement of said leaving group by a suitable nucleophile in a nucleophilic substitution reaction according to SN2 mechanism. Alternatively, said inversion of configuration of position 1 of compounds of the formula VIIb can be also obtained, for example, as described by way of example in the following examples according to subsequently specified two step procedure shown in reaction scheme 5 below. In more detail, in the first step of said procedure shown in reaction scheme 5, exemplary compounds of the formula VIIb*, in which R1, R2, R6 have the meanings indicated above, C(O)OR stands for said suitable ester group (preferably the methyl ester group) and R3, R31, R4 are hydrogen and position 1 has the R configuration, are converted by oxidation reaction into corresponding compounds of the formula XIVb. Said oxidation is likewise carried out under conditions customary per se using, for example, chloranil, atmospheric oxygen, manganese dioxide or, preferably, chromium oxides as an oxidant. Then in the second step, compounds of the formula XIVb obtained are converted by art-known reduction reaction of the keto group, preferably with metal hydride compounds or, more specifically, metal borohydrides, such as, for example, sodium borohydride, into corresponding compounds of formula VIIb**, in which position 1 has now S configuration and thus the configuration of the carbon atom in position 1 is now inverted regarding to said compounds of the formula VIIb*.

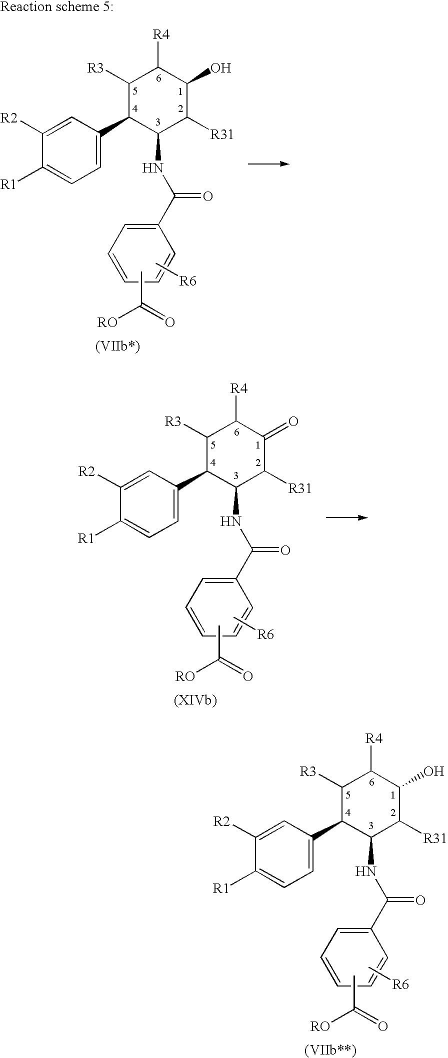

In the next reaction step of the synthesis route shown in reaction scheme 4 shown above, compounds of the formula VIIb are converted into corresponding compounds of the formula Vb by introduction of the group R51. The introduction reaction is carried out in a manner habitual per se (e.g. via alkylation or acylation reaction) or as described by way of example in the following examples.

The cyclization reaction leading to compounds of the formula IVb can be carried out, for example, as described by way of example in the following examples or analogously or similarly thereto, or as mentioned above for compounds according to embodiment a.

Compounds of the formula XIb, in which R1, R2, R3, R31 and R4 have the abovementioned meanings according to embodiment b, are either known or can be obtained, for example as shown in reaction scheme 6, by the reaction of compounds of the formula XII, in which R1 and R2 have the abovementioned meanings, with compounds of the formula XVb, in which R3, R31 and R4 have the meanings indicated above in embodiment b.

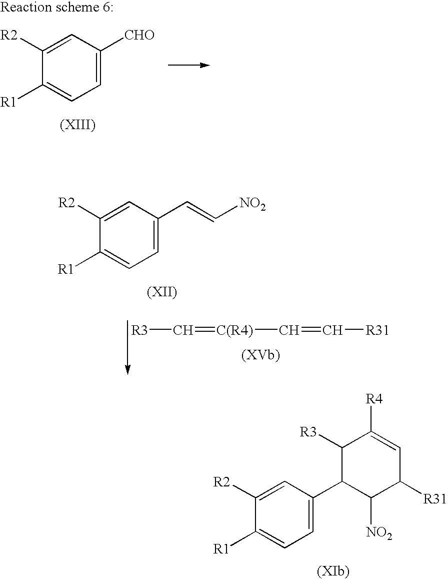

The cycloaddition is in this case carried out in a manner known to the person skilled in the art according to Diels-Alder, e.g. as described in J. Amer. Chem. Soc. 1957, 79, 6559 or in J. Org. Chem. 1952, 17, 581 or as described in the following examples.

Compounds of the formula XIb, in which the phenyl ring and the nitro group are trans to one another, can be converted such as known to the person skilled in the art into the corresponding cis compounds, e.g. as described in J. Amer. Chem. Soc. 1957, 79, 6559 or as described in the following examples.

The compounds of the formula XVb are either known or can be prepared in a known manner.

In an alternative, compounds of the formula IVb, in which R1, R2, R3, R31, R4, R51 and R6 have the meanings given above in embodiment b whereby R51 is other than hydrogen and COOR stands for a suitable ester group (particularly compounds of formula IVb, in which R1, R2, R51 and R6 have the meanings given above in embodiment b whereby R51 is other than hydrogen, and R3, R31 and R4 are all hydrogen and COOR stands for a suitable ester group) can also be obtained as shown in reaction scheme 7 and as described by way of example in the following examples.

In the first reaction step of the route outlined in reaction scheme 7, the amino group of compounds of the formula Xb is protected with an art-known protective group PG1, such as e.g. the tert-butoxycarbonyl group. The proteced compounds are subjected to hydroboration reaction to obtain over two steps corresponding compounds of formula XVIb, in which R51 is hydrogen. Said hydroboration reaction is carried out as described in the following examples using an appropriate (hydro)borating agent, such as e.g. 9-BBN, isopinocampheylborane or the like, or, particularly, borane-tetrahydrofuran (H₃B-THF), advantageously at ambient temperature.

The compounds obtained are then converted into compounds of the formula XVIb by introduction of the group R51 whereby R51 is other than hydrogen in a manner analogously as described above.

In the next reaction step of the synthesis route shown in reaction scheme 6, compounds of formula XVIb obtained are converted into corresponding compounds of the formula Vb by deprotection of the protective group PG1 and amidification with compounds of the formula VI. Said reactions are carried out in a manner habitual per se or as described in the specification of this invention or in the following examples.

If necessary, the product obtained via said hydroboration reaction or, suitably, the R51-substituted derivative thereof is purified from resulting stereo- and/or regioisomeric side products by methods known to the person skilled in the art, such as e.g. by chromatographic separation techniques.

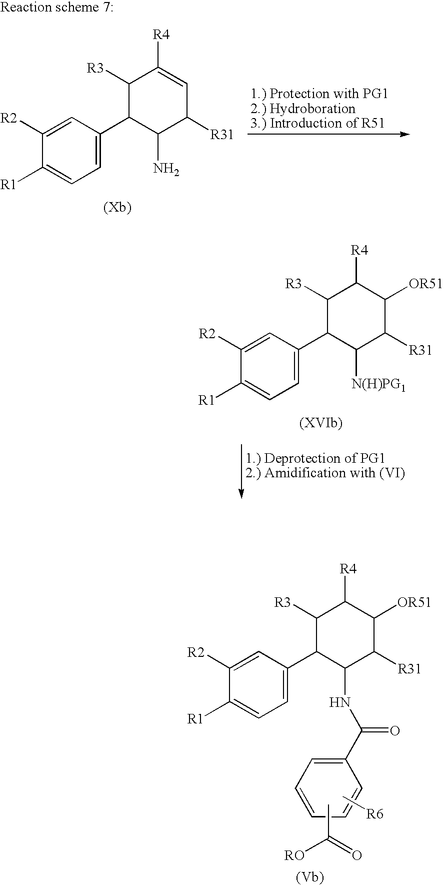

It is also known to the person skilled in the art that, if a plurality of reactive centers are present in a starting material or intermediate, it may be necessary to temporarily block one or more reactive centers with protective groups so that a reaction takes place only at the desired reactive center. A detailed description of how to use a large number of proven protective groups can be found, for example, in T. W. Greene, Protective Groups in Organic Synthesis, John Wiley & Sons, 1991 or 1999 (3^{rd} edition), or in “Protecting Groups (Thieme Foundations Organic Chemistry Series N Group)” by P. Kocienski (Thieme Medical Publishers, 2000).

The substances according to the invention are isolated and purified in a manner known per se, for example by distilling off the solvent under reduced pressure and recrystallizing the residue obtained from a suitable solvent or subjecting it to one of the customary purification methods, such as, for example, column chromatography on a suitable support material.

Salts are obtained by dissolving the free compound in a suitable solvent (e.g. a ketone, such as acetone, methyl ethyl ketone or methyl isobutyl ketone, an ether, such as diethyl ether, tetrahydrofuran or dioxane, a chlorinated hydrocarbon, such as methylene chloride or chloroform, or a low-molecular-weight aliphatic alcohol, such as ethanol or isopropanol) which contains the desired acid or base, or to which the desired acid or base is then added. The salts are obtained by filtering, reprecipitating, precipitating with a nonsolvent for the addition salt or by evaporating the solvent. Salts obtained can be converted into the free compounds, which can in turn be converted into salts, by alkalization or by acidification. In this manner, pharmacologically unacceptable salts can be converted into pharmacologically acceptable salts.

Suitably, the conversions mentioned in this invention can be carried out analogously or similarly to methods which are familiar per se to the person skilled in the art.

The person skilled in the art knows on the basis of his/her knowledge and on the basis of those synthesis routes, which are shown and described within the description of this invention, how to find other possible synthesis routes for compounds of the formula I. All these other possible synthesis routes are also part of this invention.

Having described the invention in detail, the scope of the present invention is not limited only to those described characteristics or embodiments. As will be apparent to persons skilled in the art, modifications, analogies, variations, derivations, homologisations and adaptations to the described invention can be made on the base of art-known knowledge and/or, particularly, on the base of the disclosure (e.g. the explicite, implicite or inherent disclosure) of the present invention without departing from the spirit and scope of this invention as defined by the scope of the appended claims.

The following examples serve to illustrate the invention further without restricting it. Likewise, further compounds of the formula I, whose preparation is not explicitly described, can be prepared in an analogous or similar manner or in a manner familiar per se to the person skilled in the art using customary process techniques.

Any or all of the compounds which are mentioned in the following examples as final compounds as well as their salts, N-oxides and salts of the N-oxides are a preferred subject of the present invention.

In the examples, m.p. stands for melting point, h for hour(s), min for minutes, R_{f} for rentention factor in thin layer chromatography, s.p. for sintering point, EF for empirical formula, MW for molecular weight, MS for mass spectrum, M for molecular ion, fnd. for found, calc. for calculated, other abbreviations have their meanings customary per se to the skilled person.

According to common practice in stereochemistry, the symbols RS and SR are used to denote the specific configuration of each of the chiral centers of a racemate. In more detail, for example, the term “(2RS,4aRS,10bRS)” stands for a racemate (racemic mixture) comprising the one enantiomer having the configuration (2R,4aR,10bR) and the other enantiomer having the configuration (2S,4aS,10bS).

### EXAMPLE

Final Compounds

### 1. 4-((2RS,4aRS,10bRS-2-Hydroxy-8,9-dimethoxy-1,2,3,4,4a,10b-hexahydro-phenanthridin-6-yl)-N-(2-morpholin-4-yl-ethylybenzamide

167 mg of cesium carbonate are placed in a flask. 550 mg of acetic acid (2RS,4aRS,10bRS)-8,9-dimethoxy-6-[4-(2-morpholin-4-yl-ethylcarbamoyl)-phenyl]-1,2,3,4,4a,10b-hexahydro-phenanthridin-2-yl ester (compound 36) dissolved in 10 ml of methanol are added. The solution is stirred for 16 h. The reaction mixture is adsorbed to 2 g of silica and purified by flash chromatography to give 488 mg of the title compound.

EF: C₂₃ H₃₅ N₃ O₆ MW: 493,61 MS: 494,4 (MH⁺)

Starting from the appropriate acetic acid ester compounds, which are mentioned or described explicitly below (compounds 36 to 75), or which can be prepared in a manner known to the person skilled in the art or analogously or similarly to the examples described herein, the following and also further relevant, nonexplicitly described similar compounds are obtained according to the procedure as in Example 1.

### 2. 4-((2RS,4aRS,10bRS82-Hydroxy-8,9-dimethoxy-1,2,3,4,4a,10b-hexahydro-phenanthridin-6-yl[N-(3-morpholin-4-yl-propylybenzamide

EF: C₂₉ H₃₁ N₃ O₅ MW: 507,64 MS: 508,5 (MH⁺)

### 3. 4-((2RS,4aRS,10bRS)-2-Hydroxy-8,9-dimethoxy-1,2,3,4,4a,10b-hexahydro-phenanthridin-6-yl)-N-(4-methyl-piperazin-1-yl)benzamide

EF: C₂₇ H₃₄ N₄ O₄ MW: 478,6 MS: 479,4 (MH⁺)

### 4. 4-((2RS,4aRS,10bRS-2-Hydroxy-8,9-dimethoxy-1,2,3,4,4a,10b-hexahydro-phenanthridin-6-yl)-N-morpholin-4-yl-benzamide

EF: C₂₅ H₃₁ N₃ O₅ MW: 465,55 MS: 466,4 (MH⁺)

### 5. ({1-[4-((2RS,4aRS,10bRS)-2-Hydroxy-8,9-dimethoxy-1,2,3,4,4a,10b-hexahydro-phenanthridin-6-ylyphenyl]-methanoyl}-methyl-amino)-acetic acid methyl ester

EF: C₂₆ H₃₀ N₂ O₆ MW: 466,54 MS: 467,4 (MH⁺)

### 6. 4-((2RS,4aRS,10bRS)-2-Hydroxy-8,9-dimethoxy-1,2,3,4,4a,10b-hexahydro-phenanthridin-6-yl)-N-quinolin-3-yl-benzamide

EF: C₃₁ H₂₉ N₃ O₄ MW: 507,59 MS: 508,3 (MH⁺)

### 7. 4-((2RS,4aRS,10bRS)-2-Hydroxy-8,9-dimethoxy-1,2,3,4,4a,10b-hexahydro-phenanthridin-6-yl)-N-(2-pyridin-2-yl-ethyl)-benzamide

EF: C₂₉ H₃₁ N₃ O₄ MW: 485,59 MS: 486,4 (MH⁺)

### 8. 1-[4-((2RS,4aRS,10bRS)-2-Hydroxy8,9-dimethoxy-1,2,3,4,4a,10b-hexahydro-phenanthridin-6-yliphenyl]-1-(4-pyridin-2-yl-piperazin-1-yl)-methanone

EF: C₃₁ H₃₄ N₄ O₄ MW: 526,64 MS: 527,4 (MH⁺)

### 9. 1-[4-((2RS,4aRS,10bRS)-2-Hydroxy-8,9-dimethoxy-1,2,3,4,4a,10b-hexahydro-phenanthridin-6-yl)-phenyl]-1-[4-(2-morpholin-4-yl-ethyl)-piperazin-1-yl]methanone

EF: C₃₂ H₄₂ N₄ O₅ MW: 562,72 MS: 563,4 (MH⁺)

### 10. N-Ethyl-4((2RS,4aRS,10bRS-2-hydroxy-8,9-dimethoxy-1,2,3,4,4a,10b-hexahydro-phenanthridin-6-yl)-N-(2-methoxy-ethyl)-benzamide

EF: C₂₇ H₃₄ N₂ O₅ MW: 466,58 MS: 467,3 (MH⁺)

### 11. N-Cyclopropyl-4-((2RS,4aRS,10bRS)-2-hydroxy-8,9-dimethoxy-1,2,3,4,4a,10b-hexahydro-phenanthridin-6-yl)-benzamide

EF: C₂ H₂ N₂ O₄ MW: 420,51 MS: 421,3 (MH⁺)

### 12. 2-(4-{1-[4-((2RS,4aRS,10bRS)-2-Hydroxy-8,9-dimethoxy-1,2,3,4,4a,10b-hexahydro-phenanthridin-6-yl)-phenyl]-methanoyl}-piperazin-1-yl)-1-pyrrolidin-1-yl-ethanone

EF: C₃₂ H₄₀ N₄ O₅ MW: 560,7 MS: 561,4 (MH⁺)

### 13. 2-(4-{1-[4-((2RS,4aRS,10bRS)-2-Hydroxy-8,9-dimethoxy-1,2,3,4,4a,10b-hexahydro-phenanthridin-6-yl)phenyl]methanoyl}-piperazin-1-yl)-N-pyridin-3-yl-acetamide

EF: C₃₃ H₃₇ N₅ O₅ MW: 583,69 MS: 584,4 (MH⁺)

### 14. 4-((2RS,4aRS,10bRS)-2-Hydroxy-8,9-dimethoxy-1,2,3,4,4a,10b-hexahydro-phenanthridin-6-yl)-N,N-dimethyl-benzamide

EF: C₂₄ H₂₈ N₂ O₄ MW: 408,5 MS: 409,4 (MH⁺)

### 15. 2-(4-{1-[4-((2RS,4aRS,10bRS)-2-Hydroxy-8,9-dimethoxy-1,2,3,4,4a,10b-hexahydro-phenanthridin-6-yl)-phenyl]-methanoyl}-piperazin-1-yl)-N-pyridin-2-yl-acetamide

EF: C₃₃ H₃₇ N₅ O₅ MW: 583,69 MS: 584,4 (MH⁺)

### 16. 2-(4-{1-[4-((2R,4aR,10bR)-2-Hydroxy-8,9-dimethoxy-1,2,3,4,4a,10b-hexahydro-phenanthridin-6-yiyphenyl]-methanoyl}-piperazin-1-yl)-N,N-dimethyl-acetamide

EF: C₃₀ H₃ N₄ O₅ MW: 534,66 MS: 535,4 (MH′)

### 17. 2-(4-{1-[4-((2RS,4aRS,10bRS)-2-Hydroxy-8,9-dimethoxy-1,2,3,4,4a,10b-hexahydro-phenanthridin-6-yl)-phenyl]-methanoyl}-piperazin-1-yl)-1-morpholin-4-yl-ethanone

EF: C₃₂ H₄₀ N₄ O₆ MW: 576,7 MS: 577,4 (MH⁺)

### 18. 1-[4-((2RS,4aRS,10bRS)-2-Hydroxy-8,9-dimethoxy-1,2,3,4,4a,10b-hexahydro-phenanthridin-6-yiphenyl]1-(4-pyridin-4-yl-piperazin-1-yl)-methanone

EF: C₃₁ H₃₄ N₄ O₄ MW: 526,64 MS: 527,4 (MH⁺)

### 19. 1-[4-((2RS,4aRS,10bRS)-2-Hydroxy-8,9-dimethoxy-1,2,3,4,4a,10b-hexahydro-phenanthridin-6-yl)-phenyl]-1-morpholin-4-yl-methanone

EF: C₂₆ H₃₀ N₂ O₅ MW: 450,54 MS: 451,4 (MH⁺)

### 20. 4-((2RS,4aRS,10bRS)-2-Hydroxy-8,9-dimethoxy-1,2,3,4,4a,10b-hexahydro-phenanthridin-6-yl)-N-(2-pyridin-4-yl-ethyl)-benzamide

EF: C₂₉ H₃₁ N₃ O₄ MW: 485,59 MS: 486,3 (MH⁺)

### 21. 4-((2RS,4aRS,10bRS)-2-Hydroxy-8,9-dimethoxy-1,2,3,4,4a,10b-hexahydro-phenanthridin-6-yl)-N-(2-pyridin-3-yl-ethyl)-benzamide

EF: C₂₇ H₃₂ N₃ O₄ MW: 485,59 MS: 486,4 (MH⁺)

### 22. 4-((2RS,4aRS,10bRS)-2-Hydroxy-8,9-dimethoxy-1,2,3,4,4a,10b-hexahydro-phenanthridin-6-yl)benzoic acid N′-(1-morpholin-4-yl-methanoyl)-hydrazide

EF: C₂₇ H₃₂ N₄ O₆ MW: 508,58 MS: 509,2 (MH⁺)

### 23. N-(2,6-Dimethoxy-pyridin-3-yl)-4-((2RS,4aRS,10bRS)-2-hydroxy-8,9-dimethoxy-1,2,3,4,4a,10b-hexahydro-phenanthridin-6-yl)benzamide

EF: C₂₉ H₃₁ N₃ O₆ MW: 517,59 MS: 518,3 (MH⁺)

### 24. 4-[(2RS,4aRS,10bRS)-9-(1,1-Difluoro-methoxy)-2-hydroxy-8-methoxy-1,2,3,4,4a,10b-hexahydro-phenanthridin-6-yl]-N,N-dimethyl-benzamide

EF: C₂₄ H₂₅ F₂ N₂ O₄ MW: 444,48 MS: 445,3 (MH⁺)

### 25. N-Cyclopropyl-4-[(2RS,4aRS,10bRS)-9-(1,1-difluoro-methoxy)-2-hydroxy8-methoxy-1,2,3,4,4a,10b-hexahydro-phenanthridin-6-yl]-benzamide

EF: C₂₈ H₂₆ F₂ N₂ O₄ MW: 456,49 MS: 457,3 (MH⁺)

### 26. 4-[(2RS,4aRS,10bRS)-9-(1,1-Difluoro-methoxy)-2-hydroxy-8-methoxy-1,2,3,4,4a,10b-hexahydro-phenanthridin-6-yl]-N,N-bis(2-methoxy-ethyl)-benzamide

EF: C₂₈ H₃₄ F₂ N₂ O₆ MW: 532,59 MS: 533,4 (MH⁺)

### 27. 4-[(2RS,4aRS,10bRS)-9-(1,1-Difluoro-methoxy)-2-hydroxy-8-methoxy-1,2,3,4,4a,10b-hexahydro-phenanthridin-6-yl]-N-(2-morpholin-4-yl-ethyl)-benzamide

EF: C₂₈ H₃₃ F₂ N₃ O₅ MW: 529,59 MS: 530,3 (MH⁺)

### 28. 4-[(2RS,4aRS,10bRS)-9-(1,1-Difluoro-methoxy)-2-hydroxy-8-methoxy-1,2,3,4,4a,10b-hexahydro-phenanthridin-6-yl]-N-(3-morpholin-4-yl-propyl)-benzamide

EF: C₂₉ H₃₅ F₂ N₃ O₅ MW: 543,62 MS: 544,3 (MH⁺)

### 29. 1-{4-[(2RS,4aRS,10bRS)-9-(1,1-Difluoro-methoxy)-2-hydroxy-8-methoxy-1,2,3,4,4a,10b-hexahydro-phenanthridin-6-yl]-phenyl}-1-[4(2-morpholin-4-yl-ethyl)-piperazin-1-yl]-methanone

EF: C₃₂ H₄₀ F₂ N₄ O₅ MW: 598,7 MS: 599,4 (MH⁺)

### 30. 1-{4-[(2RS,4aRS,10bRS)-9-(1,1-Difluoro-methoxy)-2-hydroxy-8-methoxy-1,2,3,4,4a,10b-hexahydro-phenanthridin-6-yl]-phenyl}-1-(4-pyridin-4-yl-piperazin-1-yl)-methanone

EF: C₃₁ H₃₂ F₂ N₄ O₄ MW: 562,62 MS: 563,3 (MH⁺) 31. 2-[4(1-{4-[(2RS,4aRS,10bRS)-9-(1,1-Difluoro-methoxy)-2-hydroxy-8-methoxy-1,2,3,4,4a,10b-hexahydro-phenanthridin-6-yl]-phenyl}-methanoyl)-piperazin-1-yl]-N-pyridin-2-yl-acetamide

EF: C₃₃ H₃₅ F₂ N₅ O₅ MW: 619,67 MS: 620,3 (MH⁺)

### 32. 2-[4-(1-{4-[(2RS,4aRS,10bRS)-9-(1,1-Difluoro-methoxy)-2-hydroxy-8-methoxy-1,2,3,4,4a,10bhexahydro-phenanthridin-6-yl]-phenyl}-methanoyl)-piperazin-1-yl]-1-morpholin-4-yl-ethanone

EF: C₃₂ H₃₈ F₂ N₄ O₆ MW: 612,68 MS: 613,4 (MH⁺)

### 33. 1-{4-[(2RS,4aRS,10bRS)-9-(1,1-Difluoro-methoxy)-2-hydroxy-8-methoxy-1,2,3,4,4a,10b-hexahydro-phenanthridin-6-yl]-phenyl}-1-pyrrolidin-1-yl-methanone

EF: C₂₆ H₂₈ F₂ N₂ O₄ MW: 470,52 MS: 471,4 (MH⁺)

### 34. 2-[4-(1-{4-[(2RS,4aRS,10bRS)-9-(1,1-Difluoro-methoxy)-2-hydroxy-8-methoxy-1,2,3,4,4a,10bhexahydro-phenanthridin-6-yl]-phenyl}-methanoyl)-piperazin-1-yl]-N,N-dimethyl-acetamide

EF: C₃₀ H₃₆ F₂ N₄ O₅ MW: 570,64 MS: 571,4 (MH⁺)

### 35. 1-{4-[(2RS,4aRS,10bRS)-9-(1,1-Difluoro-methoxy)-2-hydroxy-8-methoxy-1,2,3,4,4a,10b-hexahydro-phenanthridin-6-yl]-phenyl}-1-[4-(2-dimethylamino-ethyl)-piperazin-1-yl]-methanone

EF: C₃₀ H₃₈ F₂ N₄ O₄ MW: 556,66 MS: 557,3 (MH⁺)

### 36. Acetic acid (2RS,4aRS,10bRS)-8,9-dimethoxy-6-[4-(2-morpholin-4-yl-ethylcarbamoyl)-phenyl]-1,2,3,4,4a,10b-hexahydro-phenanthridin-2-yl ester

1000 mg of 4-((2RS,4aRS,10bRS)-2-acetoxy-8,9-dimethoxy-1,2,3,4,4a,10b-hexahydro-phenanthridin-6-yl)-benzoic acid (compound A1), 552 mg of N-ethyl-N′-(3-dimethylaminopropyl)carbodiimide hydrochloride (EDCl) and 2 mg of 4-dimethylaminopyridine are placed in a flask. 250 mg of 2-morpholin-4-yl-ethylamine are added and the solution stirred for 16 hrs. The reaction mixture is adsorbed to 3 g of silica and purified by flash chromatography to yield 715 mg of the title compound.

EF: C₃₀ H₃₇ N₃ O₆ MW: 535,65 MS: 536,3 (MH⁺)

Starting from the appropriate art-known amine compounds and the appropriate carboxylic acid starting compounds, which are mentioned or described explicitly below (compounds A1 or A2), or which can be prepared in a manner known to the person skilled in the art or analogously or similarly to the examples described herein, the following and also further relevant, non-explicitly described similar compounds are obtained according to the procedure as in Example 36.

### 37. Acetic acid (2RS,4aRS,10bRS)-8,9-dimethoxy-6-[4-(morpholin-4-ylcarbamoyl)-phenyl]-1,2,3,4,4a,10b-hexahydro-phenanthridin-2-yl ester

EF: C₂₈ H₃₃ N₃ O₆ MW: 507,59 MS: 508,4 (MH⁺)

### 38. ({1-[4((2RS,4aRS,10bRS)-2-Acetoxy-8,9-dimethoxy-1,2,3,4,4a,10b-hexahydro-phenanthridin-6-ylyphenyl)-methanoyl}-methyl-amino)-acetic acid methyl ester

EF: C₂₃ H₃ N₂ O₇ MW: 508,58 MS: 509,4 (MH⁺)

### 39. Acetic acid (2RS,4aRS,10bRS)-8,9-dimethoxy-6-[4-(3-morpholin-4-yl-propylcarbamoyl)-phenyl]-1,2,3,4,4a,10b-hexahydro-phenanthridin-2-yl ester

EF: C₃₁ H₃₉ N₃ O₆ MW: 549,67 MS: 550,4 (MH⁺)

### 40. Acetic acid (2RS,4aRS,10bRS)-8,9-dimethoxy-6-[4-(4-methyl-piperazin-1-ylcarbamoyl)-phenyl]-1,2,3,4,4a,10b-hexahydro-phenanthridin-2-yl ester

EF: C₂₉ H₃₆ N₄ O₅ MW: 520,63 MS: 521,4 (MH⁺)

### 41. Acetic acid (2RS,4aRS,10bRS)-8,9-dimethoxy-6-{4-[N′-(3-sulfamoyl-phenyl)-hydrazinocarbonyl]-phenyl}-1,2,3,4,4a,10b-hexahydro-phenanthridin-2-yl ester

EF: C₃₀ H₃₂ N₄ O₇ S MW: 592,68 MS: 593,4 (MH⁺)

### 42. Acetic acid (2RS4aRS,10bRS)-6-{4-[bis-(2-methoxy-ethyl)-carbamoyl]-phenyl}8,9-dimethoxy-1,2,3,4,4a,10b-hexahydro-phenanthridin-2-yl ester

EF: C₃₀ H₃₈ N₂ O₇ MW: 538,65 MS: 539,4 (MH⁺)

### 43. Acetic acid (2RS,4aRS,10bRS)-8,9-dimethoxy-6-[4-(quinolin-3-ylcarbamoyl)-phenyl]-1,2,3,4,4a,10b-hexahydro-phenanthridin-2-yl ester

EF: C₃₃ H₃₁ N₃ O₅ MW: 549,63 MS: 550,3 (MH⁺)

### 44. Acetic acid (2RS,4aRS,10bRS)-8,9-dimethoxy-6-[4-(2-pyridin-2-yl-ethylcarbamoyl)-phenyl]-1,2,3,4,4a,10b-hexahydro-phenanthridin-2-yl ester

EF: C₃₁ H₃₃ N₃ O₅ MW: 527,63 MS: 528,3 (MH⁺)

### 45. Acetic acid (2RS,4aRS,10bRS)-8,9-dimethoxy-6-{4-[1-(4-pyridin-2-yl-piperazin-1-yl)-methanoyl]-phenyl}-1,2,3,4,4a,10b-hexahydro-phenanthridin-2-yl ester

EF: C₃₃ H₃₆ N₄ O₅ MW: 568,68 MS: 569,3 (MH⁺)

### 46. Acetic acid (2RS,4aRS,10bRS)-6-[4-(2,3-dimethyl-imidazo[1,2-a]pyridin-7-ylcarbamoyl)-phenyl]-8,9-dimethoxy-1,2,3,4,4a,10b-hexahydro-phenanthridin-2-yl ester

EF: C₃₃ H₃₄ N₄ O₅ MW: 566,66 MS: 567,3 (MH⁺)

### 47. Acetic acid (2RS,4aRS,10bRS)-8,9-dimethoxy-6-(4-{1-[4-(2-morpholin-4-yl-ethylipiperazin-1-yl]-methanoyl}-phenyl-1,2,3,4,4a,10b-hexahydro-phenanthridin-2-yl ester

EF: C₃₄ H₄₄ N₄ O₆ MW: 604,75 MS: 605,4 (MH⁺)

### 48. Acetic acid (2RS,4aRS,10bRS)-6-(4-cyclopropylcarbamoyl-phenyl)-8,9-dimethoxy-1,2,3,4,4a,10b-hexahydro-phenanthridin-2-yl ester

EF: C₂₇ H₃₀ N₂ O₅ MW: 462,55 MS: 463,3 (MH⁺)

### 49. Acetic acid (2RS,4aRS,10bRS)-8,9-dimethoxy-6-(4-{1-[4(2-oxo-2-pyrrolidin-1-yl-ethyl)-piperazin-1-yl]methanoyl}-phenyly)-1,2,3,4,4a,10b-hexahydro-phenanthridin-2-yl ester

EF: C₃₄ H₄₂ N₄ O₆ MW: 602,74 MS: 603,4 (MH⁺)

### 50. Acetic acid (2RS,4aRS,11bRS)-6-{4-[ethyl-(2-methoxy-ethyl)-carbamoyl]-phenyl}-8,9-dimethoxy-1,2,3,4,4a,10b-hexahydro-phenanthridin-2-yl ester

EF: C₂₉ H₃₆ N₂ O₆ MW: 508,62 MS: 509,3 (MH⁺)

### 51. Acetic acid (2RS,4aRS,10bRS)-8,9-dimethoxy-6-{4-[N′-(1-pyridin-3-yl-methanoyl)-hydrazinocarbonyl]-phenyl}-1,2,3,4,4a,10b-hexahydro-phenanthridin-2-yl ester

EF: C₃₀ H₃₀ N₄ O₆ MW: 542,6 MS: 543,3 (MH⁺)

### 52. 2-({1-[4-((2RS,4aRS,10bRS)-2-Acetoxy-8,9-dimethoxy-1,2,3,4,4a,10b-hexahydrophenanthridin-6-yl)-phenyl]methanoyl}-amino)-succinic acid dimethyl ester

EF: C₃₀ H₃₄ N₂ O₉ MW: 566,61 MS: 567,3 (MH⁺)

### 53. Acetic acid (2RS,4aRS,10bRS)-8,9-dimethoxy-6-(4-{1-[N′-(1-pyridin-4-yl-methanoyl)-hydrazino]-methanoyl}-phenyly)-1,2,3,4,4a,10b-hexahydro-phenanthridin-2-yl ester

EF: C₃₀ H₃₀ N₄ O₆ MW: 542,6 MS: 543,3 (MH⁺)

### 54. Acetic acid (2RS,4aRS,10bRS)-8,9-dimethoxy-6-[4-([1,7]naphthyridin-8-ylcarbamoyl)-phenyl]-1,2,3,4,4a,10b-hexahydro-phenanthridin-2-yl ester

EF: C₃₂ H₃₀ N₄ O₅ MW: 550,62 MS: 551,3 (MH⁺)

### 55. Acetic acid (2RS,4aRS,10bRS)-8,9-dimethoxy-6-(4-{1-[4-(pyridin-2-ylcarbamoylmethyl)-piperazin-1-yl]methanoyl}-phenyl)-1,2,3,4,4a,10b-hexahydro-phenanthridin-2-yl ester

EF: C₃₅ H₃₉ N₅ O₆ MW: 625,73 MS: 626,3 (MH⁺)

### 56. Acetic acid (2RS,4aRS,10bRS)-6-(4-dimethylcarbamoyl-phenyl)-8,9-dimethoxy-1,2,3,4,4a,10b-hexahydro-phenanthridin-2-yl ester

EF: C₂₆ H₃₀ N₂ O₅ MW: 450,54 MS: 451,3 (MH⁺)

### 57. Acetic acid (2RS,4aRS,10bRS)-6-{4-[1-(4-dimethylcarbamoylmethyl-piperazin-1-yl)-methanoyl]-phenyl}-8,9-dimethoxy-1,2,3,4,4a,10b-hexahydro-phenanthridin-2-yl ester

EF: G₃₂ H₄ N₄ O₆ MW: 576,7 MS: 577,3 (MH⁺)

### 58. Acetic acid (2RS,4aRS,10bRS)-8,9-dimethoxy-6-(4-{1-[4-(2-morpholin-4-yl-2-oxo-ethyl)-piperazin-1-yl)-methanoyl}-phenyl)-1,2,3,4,4a,10b-hexahydro-phenanthridin-2-yl ester

EF: C₃₄ H₄₂ N₄ O₇ MW: 618,74 MS: 619,4 (MH⁺)

### 59. Acetic acid (2RS,4aRS,10bRS)-8,9-dimethoxy-6-{4-[1-(4-pyridin-4-yl-piperazin-1-yl)-methanoyl]-phenyl}-1,2,3,4,4a,10b-hexahydro-phenanthridin-2-yl ester

EF: C₃₃ H₃₆ N₄ O₅ MW: 568,68 MS: 569,4 (MH⁺)

### 60. Acetic acid (2RS,4aRS,10bRS)-8,9-dimethoxy-6-[4-(1-morpholin-4-yl-methanoylyphenyl)-phenyl]-1,2,3,4,4a,10b-hexahydro-phenanthridin-2-yl ester

EF: C₂₈ H₃₂ N₂ O₆ MW: 492,58 MS: 493,4 (MH⁺)

### 61. Acetic acid (2RS,4aRS,10bRS)-8,9-dimethoxy-6-[4-(2-pyridin-4-yl-ethylcarbamoyl)-phenyl]-1,2,3,4,4a,10b-hexahydro-phenanthridin-2-yl ester

EF: C₃₁ H₃₃ N₃ O₅ MW: 527,63 MS: 528,3 (MH⁺)

### 62. Acetic acid (2RS,4aRS,10bRS)-8,9-dimethoxy-6-[4-(2-pyridin-3-yl-ethylcarbamoyl)-phenyl]-1,2,3,4,4a,10b-hexahydro-phenanthridin-2-yl ester

EF: C₃₁ H₃₃ N₃ O₅ MW: 527,63 MS: 528,3 (MH⁺)

### 63. Acetic acid (2RS,4aRS,10bRS)-8,9-dimethoxy-6-{4-[N′-(1-morpholin-4-yl-methanoyl)-hydrazinocarbonyl]-phenyl}-1,2,3,4,4a,10b-hexahydro-phenanthridin-2-yl ester

EF: C₂₉ H₃₄ N₄ O₇ MW: 550,62 MS: 551,2 (MH⁺)

### 64. Acetic acid (2RS,4aRS,10bRS)-6-[4-(2,6-dimethoxy-pyridin-3-ylcarbamoyl)-phenyl]-8,9-dimethoxy-1,2,3,4,4a,10b-hexahydro-phenanthridin-2-yl ester

EF: C₃₁ H₃₃ N₃ O₇ MW: 559,62 MS: 560,3 (MH⁺)

### 65. Acetic acid (2RS,4aRS,10bRS)-9-(difluoro-methoxy)-6-(4-dimethylcarbamoyl-phenyl)-8-methoxy-1,2,3,4,4a,10b-hexahydro-phenanthridin-2-yl ester

EF: C₂₆ H₂₈ F₂ N₂ O₆ MW: 486,52 MS: 487,3 (MH⁺)

### 66. Acetic acid (2RS,4aRS,10bRS)-6-(4-yclopropylcarbamoyl-phenyl)-9-(1,1-difluoro-methoxy)-8-methoxy-1,2,3,4,4a,10b-hexahydro-phenanthridin-2-yl ester

EF: C₂₇ H₂₈ F₂ N₂ O₅ MW: 498,53 MS: 499,3 (MH⁺)

### 67. Acetic acid (2RS,4aRS,10bRS)-6-{4-[bis-(2-methoxy-ethyl)-carbamoyl]-phenyl}-9-(difluoro-methoxy)-8-methoxy-1,2,3,4,4a,10b-hexahydro-phenanthridin-2-yl ester

EF: C₃₀ H₃₆ F₂ N₂ O₇ MW: 574,63 MS: 575,3 (MH⁺)

### 68. Acetic acid (2RS,4aRS,10bRS)-9-(1,1-difluoro-methoxy)-8-methoxy-6-[4-(2-morpholin-4-yl-ethylcarbamoyl)-phenyl]-1,2,3,4,4a,10b-hexahydro-phenanthridin-2-yl ester

EF: C₃₀ H₃₅ F₂ N₃ O₆ MW: 571,63 MS: 572,3 (MH⁺)

### 69. Acetic acid (2RS,4aRS,10bRS)-9-(1,1-difluoro-methoxy)-8-methoxy-6-[4-(3-morpholin-4-yl-propylcarbamoyl)-phenyl]-1,2,3,4,4a,10b-hexahydro-phenanthridin-2-yl ester

EF: C₃₁ H₃₇ F₂ N₃ O₆ MW: 585,65 MS: 586,3 (MH⁺)

### 70. Acetic acid (2RS,4aRS,10bRS)-9-(1,1-difluoro-methoxy)-8-methoxy-6-(4-{1-[4-(2-morpholin-4-yl-ethyl)-piperazin-1-yl]-methanoyl}-phenyly)-1,2,3,4,4a,10b-hexahydro-phenanthridin-2-yl ester

EF: C₃₄ H₄₂ F₂ N₄ O₆ MW: 640,73 MS: 641,4 (MH⁺)

### 71. Acetic acid (2RS,4aRS,10bRS)-9-(1,1-difluoro-methoxy)-8-methoxy-6-{4-[1-(4-pyridin-4-yl]-piperazin-1-yl)-methanoyl]-phenyl}-1,2,3,4,4a,10b-hexahydro-phenanthridin-2-yl ester

EF: C₃₃ H₃₄ F₂ N₄ O₅ MW: 604,66 MS: 605,4 (MH⁺)

### 72. Acetic acid (2RS,4aRS,10bRS)-9-(1,1-difluoro-methoxy)-8-methoxy-6-(4-{1-[4(pyridin-2-ylcarbamoylmethyl)-piperazin-1-yl]-methanoyl}-phenyl-1,2,3,4,4a,10b-hexahydro-phenanthridin-2-yl ester

EF: C₃₅ H₃₇ F₂ N₅ O6 MW: 661,71 MS: 662,4 (MH⁺)

### 73. Acetic acid (2RS,4aRS,10bRS)-9-(1,1-difluoro-methoxy)-8-methoxy-6-(4-{1-[4-(2-morpholin-4-yl-2-oxo-ethyl)-piperazin-1-yl]-methanoyl}-phenyl)-2,3,4,4a,10b-hexahydro-phenanthridin-2-yl ester

EF: C₃₄ H₄₀ F₂ N₄ O₇ MW: 654,72 MS: 655,4 (MH⁺)

### 74. Acetic acid (2RS,4aRS,10bRS)-9-(1,1-difluoro-methoxy)-8-methoxy-6-[4-(1-pyrrolidin-1-yl-methanoyl)-phenyl]-1,2,3,4,4a,10b-hexahydro-phenanthridin-2-yl ester

EF: C₂₈ H₃₀ F₂ N₂ O₅ MW: 512,56 MS: 513,4 (MH⁺)

### 75. Acetic acid (2R,4aR,10bR)-9(1,1-difluoro-methoxy)-6-(4-{1-[4-(2-dimethylamino-ethyl)-piperazin-1-yl]-methanoyl}-phenyl)-8-methoxy-1,2,3,4,4a,10b-hexahydro-phenanthridin-2-yl ester

EF: C₃₂ H₄₀ F₂ N₄ O₅ MW: 598,7 MS: 599,3 (MH⁺)

The following compounds are obtained from the corresponding racemates by chromatographical separation, which can be afforded with one or more of the following columns:
CHIRALPAK® AD-H 5 μm (250×20 mm), 25° C.,
heptane/2-propanol/diethylamine=90/10/0.1; 20 ml/min, detection at 340 nm;
CHIRALPAK® AD 20 μm (285×110 mm), 30° C., acetonitrile/isopropanol=95:5; 570 ml/min, detection at 250 nm or 280 nm;
CHIRALPAK® AD 20 μm (250×50 mm), ambient temperature, heptane/isopropanol=95:5, 120 ml/min, detection at 330 nm; or
CHIRALPAK® 50801 20 μm (250×50 mm), 25° C., methanol, 120 ml/min, detection at 330 nm.

### 76. N-(2,6-Dimethoxy-pyridin-3-yl)-4-((2R,4aR,10bR)-2-hydroxy-8,9-dimethoxy-1,2,3,4,4a,10bhexahydro-phenanthridin-6-yl)-benzamide

EF: C₂₉ H₃₁ N₃ O₆ MW: 517,59 MS: 518,4 (MH⁺) [a]²_{D}=−50°

### 77. N-(2,6-Dimethoxy-pyridin-3-yl)-4-((2S,4aS,10bS)-2-hydroxy-8,9-dimethoxy-1,2,3,4,4a,10b-hexahydro-phenanthridin-6-yl)-benzamide

EF: C₂₉ H₃₁ N₃ O₆ MW: 517,59 MS: 518,4 (MH⁻)

### 78. N-Cyclopropyl-4-[(2R,4aR,10bR)-9-(1,1-difluoro-methoxy)-2-hydroxy-8-methoxy-1,2,3,4,4a,10b-hexahydro-phenanthridin-6-yl]-benzamide

EF: C₂₅ H₂₆ F2 N₂ O₄ MW: 456,49 MS: 457,3 (MH⁺) [a]²⁰_{D}=−95°

### 79. N-Cyclopropyl-4-[(2S,4aS,10bS)-9-(1,1-diluoro-methoxy)-2-hydroxy-8-methoxy-1,2,3,4,4a,10b-hexahydro-phenanthridin-6-yl]-benzamide

EF: C₂₅ H₂₆ F₂ N₂ O₄ MW: 456,49 MS: 457,4 (MH⁺)

Starting from the appropriate acetic acid ester compounds, which are mentioned or described explicitly below (compounds 86 to 92), the following compounds 80 to 86 are obtained according to the procedure as in Example 1.

### 80. N-Cyclopropyl-4-((2R,4aR,10bR)-9-ethoxy-2-hydroxy-8-methoxy-1,2,3,4,4a,10b-hexahydro-phenanthridin-6-yl)-benzamide

C₂₆H₃₀N₂O₄Calc.: 434,54

### 81. N-Cyclobutyl-4-((2R,4aR,10bR)-9-ethoxy-2-hydroxy-8-methoxy-1,2,3,4,4a,10b-hexahydro-phenanthridin-6-yl)-benzamide C₂₇H₃₈N₂O₄ Calc.: 448,57 Found (MH⁺): 449,3

### 82. 4-((2R,4aR,10bR)-9-Ethoxy-2-hydroxy-8-methoxy-1,2,3,4,4a,10b-hexahydro-phenanthridin-6-yl)-N,N-diisopropyl-benzamide

C₂₉H₃₈N₂O₄ Calc.: 478,64 Found (MH⁺): 479,3

### 83. N-Cyclopropyl-3-((2R,4aR,10bR)-9-ethoxy-2-hydroxy-8-methoxy-1,2,3,4,4a,10b-hexahydro-phenanthridin-6-yl)-benzamide

C₂₃H₃₀N₂O₄ Calc.: 434,54 Found (MH⁺): 435,3

### 84. N-Cyclobutyl-3-((2R,4aR,10bR)-9-ethoxy-2-hydroxy-8-methoxy-1,2,3,4,4a,10b-hexahydro-phenanthridin-6-yl)-benzamide

C₂₇H₃₂N₂O₄ Calc.: 448,57

### 85. 3-((2R,4aR,10bR)-9-Ethoxy-2-hydroxy-8-methoxy-1,2,3,4,4a,10b-hexahydro-phenanthridin-6-yl)-N,N-diisopropyl-benzamide

C₂₉H₃₈N₂O₄ Calc.: 478,64 Found (MH⁺): 479,3

### 86. N-Cyclopropyl-4-((3S,4aR,10bR)-9-ethoxy-3-hydroxy-8-methoxy-1,2,3,4,4a,10b-hexahydro-phenanthridin-6-yl)benzamide

C₂₆H₃₀N₂O₄ Calc.: 434.54

Starting from the appropriate art-known amine compounds and the appropriate carboxylic acid starting compounds which are mentioned or described explicitly below, or which can be prepared in a manner known to the person skilled in the art or analogously or similarly to the examples described herein, the following are obtained according to the procedure as in Example 36.

### 87. Acetic acid (2R,4aR,10bR)-6-(3-cyclopropylcarbamoyl-phenyl)-9-ethoxy-8-methoxy-1,2,3,4,4a,10b-hexahydro-phenanthridin-2-yl ester

C₂₅H₃₂N₂O₅ Calc.: 476,58 Found (MH⁺): 477,3

### 88. Acetic acid (2R,4aR,10bR)-6-(3-cyclobutylcarbamoyl-phenyl)-9-ethoxy-8-methoxy-1,2,3,4,4a,10b-hexahydro-phenanthridin-2-yl ester

C₂₉H₃₄N₂O₅ Calc.: 490,60

### 89. Acetic acid (2R,4aR,10bR)-6-(3-diisopropylcarbamoyl-phenyl)-9-ethoxy-8-methoxy-1,2,3,4,4a,10b-hexahydro-phenanthridin-2-yl ester

C₃₁H₄₀N₂O₅ Calc.: 520,67 Found (MH⁺): 521,4

### 90. Acetic acid (2R,4aR,10bR)-6-(4-cyclopropylcarbamoyl-phenyl)-9-ethoxy-8-methoxy-1,2,3,4,4a,10b-hexahydro-phenanthridin-2-yl ester

C₂₈H₃₂N₂O₅ Calc.: 476,58 Found (MH⁺): 477,3

### 91. Acetic acid (2R,4aR,10bR)-6-(4-cyclobutylcarbamoyl-phenyl)-9-ethoxy-8-methoxy-1,2,3,4,4a,10b-hexahydro-phenanthridin-2-yl ester

C₂₉H₃₄N₂O₅ Calc.: 490,60 Found (MH⁺): 491,3

### 92. Acetic acid (2R,4aR,10bR)-6-(4-diisopropylcarbamoyl-phenyl)-9-ethoxy-8-methoxy-1,2,3,4,4a,10b-hexahydro-phenanthridin-2-yl ester

C₂₅H₂₈N₂O₅ Calc.: 436,51 Found (MH⁺): 521,4

### 93. Acetic acid (3S,4aR,10bR)-6-(4-cyclopropylcarbamoyl-phenyl)-9-ethoxy-8-methoxy-1,2,3,4,4a,10b-hexahydro-phenanthridin-3-yl ester

C₂₈H₃₂N₂O₅ Calc.: 476,58

Starting Compounds

### A1. 4-((2RS,4aRS,10bRS)-2-Acetoxy-8,9-dimethoxy-1,2,3,4,4a,10b-hexahydro-phenanthridin-6-yl)-benzoic acid

8.1 g of (2RS,4aRS,10bRS)-6-(4-carboxyphenyl)-8,9-dimethoxy-(1,2,3,4,4a,10b)-hexahydrophenanthridin-2-ol (compound B1) are suspended in 35 ml of dichloromethane and 40 ml of acetyl chloride are added dropwise. After stirring for 1 h at room temperature, the mixture is concentrated and the residue is dissolved in aqueous 1 M disodium hydrogenphosphate solution at pH 6-7. Under stirring concentrated hydrochloric acid is added, the resulting precipitate is filtered off and dried in vacuo to give 4.65 g of the title compound as beige hydrochloride salt.

The free acid is obtained by dissolving the hydrochloride salt in water at pH 6-7, removal of the solvent in vacuo, leaching the resulting yellowish residue with boiling chloroform and concentration of the obtained chloroform solution.

EF: C₂₄H₂₅NO₆; MW: 423.47 MS: 424.3 (MH⁺)

Further appropriate phenyl-carboxylic acid starting compounds can be prepared in a manner known to the person skilled in the art and analogously or similarly to the examples described herein according to the individual steps of the synthesis routes described and used herein.

### A2. 4-((2RS,4aRS,10bRS)-2-Acetoxy-9-(1,1-difluoro-methoxy)-8-methoxy-1,2,3,4a,10b-hexahydro-phenanthridin-6-yl)-benzoic acid

The title compound is obtained in two steps starting from compound B2 by saponification analogously as described in Example B1 followed by acetylation of obtained intermediate (2RS,4aRS,10bRS)-6-(4-carboxyphenyl)-9-(1,1-difluoro-methoxy)-8-methoxy-(1,2,3,4,4a,10b)-hexahydrophenanthridin-2-ol analogously as described in Example A1.

EF: C₂₄H₂₃F₂NO₆; MW: 459.45 MS: 460.3 (MH⁺)

Using similar procedures to those described to obtain compound A1, but with suitable choice of starting materials which are described herein or which are accessible in analogy to the described ones, the following compounds can be prepared:

### A3. 4-((2RS,4aRS,10bRS)-2-Acetoxy-9-ethoxy-8-methoxy-1,2,3,4,4a,10b-hexahydro-phenanthridin-6-yl)-benzoic acid

### A4. 4-((2RS,4aRS,10bRS)-2-Acetoxy-9-(2,2-difluoroethoxy)-8-methoxy-1,2,3,4,4a,10b-hexahydro-phenanthridin-6-yl)-benzoic acid

### A5. 3-((2RS,4aRS,10bRS)-2-Acetoxy-8,9dimethoxy-1,2,3,4,4a,10b-hexahydro-phenanthridin-6-yl)-benzoic acid

### A6. 3-((2RS,4aRS,10bRS)-2-Acetoxy-9-(1,1-difluoro-methoxy)-8-methoxy-1,2,3,4,4a,10b-hexahydro-phenanthridin-6-yl)-benzoic acid

### A7. 3-((2RS,4aRS,10bRS)-2-Acetoxy-9-ethoxy-8-methoxy-1,2,3,4,4a,10b-hexahydro-phenanthridin-6-yl)-benzoic acid

### A8. 3-((2RS,4aRS,10bRS)-2-Acetoxy-9-(2,2-difluoroethoxy)-8-methoxy-1,2,3,4,4a,10b-hexahydro-phenanthridin-6-yl)-benzoic acid

### B1. (2RS,4aRS,10bRS)-6-(4-carboxyphenyl)-8,9-dimethoxy-(1,2,3,4,4a,10b)-hexahydro-phenanthridin-2-ol

A solution of 290 mg of acetic acid (2RS,4aRS,10bRS)-6-(4-methoxycarbonylphenyl)-8,9-dimethoxy-(1,2,3,4,4a,10b)-hexahydrophenanthridin-2-yl ester (compound C1) in 10 ml of isopropanol is treated dropwise with aqueous lithium hydroxide solution to adjust to pH 10. Stirring is continued for 72 h, the reaction mixture is neutralized with phosphate buffer solution and extracted with dichloromethane. The aqueous layer is concentrated and the residue is leached with a boiling mixture of ethyl acetate and methanol. The organic solvents are removed to obtain 90 mg of the title compound as a yellowish foam.

EF: C₂₂H₂₃NO₅; MW: 381.43 MS: 382.4 (MH⁺) M.p.: 172-183° C.

Alternative Procedure:

A solution of 5.68 g of acetic acid (2RS,4aRS,10bRS)-6-(4-methoxycarbonylphenyl)-8,9-dimethoxy-(1,2,3,4,4a,10b)-hexahydrophenanthridin-2-yl ester (compound C1) in 250 ml of methanol is treated at boiling temperature with a solution of 2.0 g of sodium hydroxide in 15 ml of water comprising a catalytic amount of hydrogen peroxide (30% strength). Stirring is continued for 1.5 h under reflux, the reaction mixture is cooled and treated with halfconcentrated aqueous hydrochloric acid to adjust to pH 6-7. The solvents are evaporated and the residue is dried in vacuo to obtain 8.1 g of a yellowish solid, which can be used without further purification in the next step. The free acid is obtained by leaching the residue with boiling chloroform and concentration of the resulting chloroform solution.

### B2. 4-[(2RS,4aRS,10bRS)-2-Acetoxy-9-(1,1-difluoro-methoxy)-8-methoxy-1,2,3,4,4a,10b-hexahydro-phenanthridin-6-yl]-benzoic acid methyl ester

500 mg of N-{(1RS,2RS,4RS3)-4-acetoxy-2-[3-(1,1-difluoro-methoxy)-4-methoxy-phenyl]-cyclohexyl}-terephthalamic acid methyl ester (compound C2) are dissolved in 2 ml of phosphorus oxychloride and heated for 4.5 h at 100° C. After cooling to room temperature the sample is diluted with 10 ml of dichloromethane and added dropwise to an aqueous sodium hydroxide solution. The water layer is extracted twice with dichloromethane. The solvent is removed and the crude product purified by chromatography on silica gel to give 310 mg of the title compound as a colourless foam.

EF: C₂₅H₂₅F₂NO₆; MW: 473.48 MS: 474.2 (MH⁺)

### C1. Acetic acid (2RS,4aRS,10bRS)-6-(4-methoxycarbonylphenyl)-8,9-dimethoxy-(1,2,3,4,4a,10b)-hexahydrophenanthridin-2-yl ester

10.8 g of phosphorus pentachloride are suspended in 170 ml of isopropyl acetate, 8.1 g of acetic acid (1RS,3RS,4RS)-4-{[1-(4-methoxycarbonylphenyl)methanoyl]amino}-3-(3,4-dimethoxyphenyl)cyclohexyl ester (compound D1) dissolved 100 ml are added and the mixture is stirred. When reaction is complete, a mixture of 100 ml of triethylamine and 100 ml of isopropyl acetate is added dropwise at 0° C. After diluting with 80 ml water at 0° C. and phase separation, the aqueous phase is extracted three times with each 60 ml of dichloromethane. The organic phases are dried using magnesium sulfate. After concentrating, the residue is recrystallized from ethyl acetate/cyclohexane to give 5.68 g of the title compound.

EF: C₂₅H₂₇NO₆; MW: 437.50 MS: 438.3 (MH⁺) R_{f}=0.62 (petroleum ether/ethyl acetate/triethylamine=6/3/1) M.p.: 184-185° C.

Starting from the appropriate starting compounds mentioned below or obtainable for the skilled person in a manner analogous to the described examples, further relevant starting compounds can be obtained according to the abovedescribed cyclization reactions or analogously or similarly thereto. If necessary, the cyclization reaction can be carried out in the presence of a catalytic amount of a Lewis acid such e.g. tin tetrachloride.

### C2. N-{(1RS,2RS,4RS)-4-Acetoxy-2-[3-(1,1-difluoro-methoxy)-4-methoxy-phenyl]-cyclohexyl}-terephthalamic acid methyl ester

The title compound is prepared analogously as described in Example D1 starting from compound D2.

EF: C₂₅H₂₇F₂NO₇; MW: 491.49 MS: 492.0 (MH⁺)

Further starting compounds can be obtained from appropriate compounds mentioned below analogously or similarly to Example D1.

### D1. Acetic acid (1RS,3RS,4RS)-4-{[1-(4-methoxycarbonylphenyl)methanoyl]amino}-3-(3,4-dimethoxyphenyl)cyclohexyl ester

1.6 g of acetic acid (1RS,3RS,4RS)-4-amino-3-(3,4-dimethoxyphenyl)cyclohexyl ester (compound E1) are dissolved in 30 ml of dichloromethane. 982 mg (5.45 mmol) of terephthalic acid monomethyl ester and 1.25 g (6.74 mmol) of N-ethyl-N′-(3-dimethylaminopropyl)carbodiimide hydrochloride are added successively under stirring. After 3 h further 18 mg (0.1 mmol) of terephthalic acid monomethyl ester are added. After 15 h the reaction is treated with aqueous hydrochloric acid and extracted several times with dichloromethane. After evaporation of the combined organic phases, the crude product is crystallized from ethyl acetate/cyclohexane to give 1.87 g (73% of theory) of the title compound as colourless solid.

EF: C₂₅H₂₉NO₇; MW: 455.51 MS: 456.2 (MH⁺) R_{f}=0.69 (ethyl acetate/triethylamine=9/1)

### D2. Acetic acid (1RS,3RS,4RS)-4-amino-3-[3-(1,1-difluoro-methoxy)-4-methoxy-phenyl]-cyclohexyl ester

The title compound is prepared analogously as described in Example E1 starting from compound E2.

EF: C₁₆H₂₁F₂NO₄; MW: 329.35 MS: 330.0 (MH⁺)

### D3. Acetic acid (1RS,3RS,4RS)-4-amino-3-(3-ethoxy-4-methoxy-phenyl)-cyclohexyl ester

The title compound is prepared analogously as described in Example E1 starting from the appropriate starting compound obtainable analogously as described in the examples below.

EF: C₁₇H₂₅NO₄; MW: 307.39 MS: 308.0 (MH⁺)

### D3a. Acetic acid (1R,3R,4R)-4-amino-3-(3-ethoxy-4-methoxy-phenyl)-cyclohexyl ester

24.0 g (55.0 mmol) of the pyroglutamate of the title compound (compound D3b) are suspended in 150 ml of water, 100 ml of dichloromethane are added, then saturated KHCO₃-solution until the gas evolution ceased. After phase separation, reextraction of the water layer and drying the combined organic layers with sodium sulfate the solvent is removed to give 16.9 g of the salt-free title compound.

Analytical Column Chromatography (CHIRALPAK AD-H 250×4.6 mm 5μ No.ADH0CE-DB030, Eluent: n-Hexan/iPrOH=80/20 (v/v)+0.1% Diethylamine): Retention Time: 6.54 min

D3b. Acetic acid (1R,3R,4R)-4-amino-3-(3-ethoxy-4-methoxy-phenylycyclohexyl ester, salt with L-pyroglutamic acid

Solution A: 55.2 g (180 mmol) of racemic acetic acid (1RS,3RS,4RS)-4-amino-3-(3-ethoxy-4-methoxy-phenyl)-cyclohexyl ester (compound D3) are dissolved in 540 ml of isopropyl acetate.

Solution B: 18.6 g (144 mmol) of L-pyroglutamic acid are dissolved in 260 ml of isopropanol under heating, then 290 ml of isopropyl acetate is added carefully.

Solution B is added to solution A and left for 48 hours. The solid is filtered off and washed with a little isopropyl acetate to give after drying 32.48 g colorless crystals with a ratio of the enantiomers of 97:3 in favour of the title compound.

M.p.: 165-167° C.

### D4. Acetic acid (1RS,3RS,4RS)-4-amino-3-[4-(1,1-difluoro-methoxy)-3-methoxy-phenyl]-cyclohexyl ester

The title compound is prepared analogously as described in Example E1 starting from the appropriate starting compound obtainable analogously as described in the examples below.

EF: C₁₆H₂₁F₂NO₄; MW: 329.35 MS: 330.0 (MH⁺)

### D5. Acetic acid (1RS,3RS,4RS)-4-amino-3-[3-(2,2-difluoro-ethoxy)-4-methoxy-phenyl]-cyclohexyl ester

The title compound is prepared analogously as described in Example E1 starting from the appropriate starting compound obtainable analogously as described in the examples below.

### D5a. Acetic acid (1R,3R,4R)-4-amino-3-[3-(2,2-difluoro-ethoxy)-4-methoxy-phenyl]-cyclohexyl ester

The title compound is obtained from its pyroglutamate salt (compound D5b) analogously as described for compound D3a using sodium hydrogencarbonate solution.

### D5b. Acetic acid (1R,3R,4R)-4-amino-3-[3-(2,2-difluoro-ethoxy)-4-methoxy-phenyl]-cyclohexyl ester, salt with L-pyroglutamic acid

343 mg (1.00 mmol) of acetic acid (1RS,3RS,4RS)-4-amino-3-[3-(2,2-difluoro-ethoxy)-4-methoxy-phenyl]-cyclohexyl ester (compound D5) are dissolved in 3 ml of isopropanol. A solution of 103 mg (0.80 mmol) of L-pyroglutamic acid in 2 ml of isopropanol is added. After filtering and drying 162 mg of the pyroglutamate are isolated with an enantiomeric ratio of 97:3 in favour of the title compound.

### D6. Acetic acid (1SR,3RS,4RS)-3-amino-4-(3-ethoxy-4-methoxy-phenyl)-cyclohexyl ester

3.0 g (7.36 mmol) of acetic acid (1SR,3RS,4RS)-3-tert-butoxycarbonylamino4-(3-ethoxy-4-methoxy-phenyl)-cyclohexyl ester (compound E6) are dissolved in 6 ml of 4 M HCl in dioxane and stirred for 30 min. After removal of the solvent the residue is dissolved in dichloromethane and 25 ml of sat. NaHCO₃ solution are added carefully. After phase separation, reextraction of the water layer and drying of the combined organic layers (Na₂SO₄) the solvent is removed to give 2.25 g of the title compound.

EF: C17 H25 N O4; MW: 307.39 MS: 308.1 (MH⁺)

### D7. Acetic acid (1SR,3RS,4RS)-3-amino-4-(3,4-dimethoxy-phenyl)-cyclohexyl ester

The title compound can be obtained from compound E7 analogously as described for compound D6.

### E1. Acetic acid (1RS,3RS,4RS)-4-amino-3-(3,4-dimethoxyphenyl)cyclohexyl ester

A solution of 10.37 g of acetic acid (1RS,3RS,4RS)-3-(3,4dimethoxyphenyl)-4-nitrocyclohexyl ester (compound F1) in 240 ml of ethanol is added to a zinc-copper couple, prepared from 16.8 g of zinc powder and 920 mg of copper (II) acetate monohydrate in acetic acid, the resulting suspension is refluxed and treated with 26 ml of acetic acid, 3.2 ml of water and 26 ml of ethanol. The resulting mixture is refluxed for further 15 min. The precipitate is filtered off with suction and the solvent is removed. Chromatographical purification on silica gel using a mixture of petroleum ether/ethyl acetate/triethylamine in the ratio 2/7/1 and concentration of the corresponding eluate fractions afford 5.13 g (55% of theory) of the title compound as a pale brown oil.

R_{f}=0.35 (petroleum ether/ethyl acetate/triethylamine=2/7/1)

### E2. Acetic acid (1RS,3RS,4RS)-3-[3-(1,1-difluoro-methoxy)-4-methoxy-phenyl]-4-nitrocyclohexyl ester

The title compound is prepared analogously as described in Example F1 starting from compound F2.

Starting from the starting compounds mentioned below, the following are obtained according to the procedure as in Example F1.

### E3. Acetic acid (1RS,3RS,4RS)-3-(3-ethoxy-4-methoxy-phenyl)-4-nitrocyclohexyl ester

### E4. Acetic acid (1RS,3RS,4RS)-3-[4-(1,1-difluoro-methoxy)-3-methoxy-phenyl]-4-nitrocyclohexyl ester

### E5. Acetic acid (1RS,3RS,4RS)-3-(3-(2,2difluoro-ethoxy)-4-methoxy-phenyl]-4-nitrocyclohexyl ester

### E6. Acetic acid (1SR,3RS,4RS)-3-tert-butoxycarbonylamino-4-(3-ethoxy-4-methoxy-phenyl)-cyclohexyl ester

22.64 g (65 mmol) of [(1RS,6RS)-6-(3-ethoxy-4-methoxy-phenyl)-cyclohex-3-enyl]-carbamic acid tert-butyl ester (compound F6) are dissolved in 180 ml of THF and 50 ml of BH₃ (1 M solution in THF) are added dropwise (30 min). After stirring for 2 h the mixture is cooled using an ice bath and a mixture of 30 ml of H₂O₂ (30%) and 60 ml of aqueous NaOH (3 M) is added. The mixture is stirred for 30 min at room temperature. 400 ml of water and 200 ml of dichloromethane are added. After phase separation, reextraction of the water layer and drying of the combined organic layers (Na₂SO₄) the solvent is removed and the crude product (23.42 g, mixture of the two mentioned regioisomers ˜2:1 in favour of the title compound) is used directly without further purification.

The crude material from above then is dissolved in 50 ml of pyridine. 50 mg of 4-dimethylaminopyridine and 60 ml of acetic anhydride are added and the mixture stirred for 90 min at 100° C. The solvents and the acetic anhydride are removed (sat. NaHCO₃ solution). Purification by means of chromatography yields 9.4 g of the title compound as colorless foam.

EF: C22 H33 N O6; MW: 407.51 MS: 308.1 (MH⁺-Boc), 407.8 (MH⁺), 430.1 (Mna⁺)

### E7. Acetic acid (1SR,3RS,4RS)-3-tert-butoxycarbonylamino-4-(3,4-dimethoxy-phenyl)-cyclohexyl ester

The title compound can be obtained from compound F7 analogously as described for compound E6.

### F1. Acetic acid (1RS,3RS,4RS)-3-(3,4-dimethoxyphenyl)-4-nitrocyclohexyl ester

10.18 g of (1RS,3RS,4RS)-3-(3,4-dimethoxyphenyl)-4-nitrocyclohexanol (compound G1) are dissolved in 100 ml of acetic anhydride and the solution is heated to 100° C. for 1-2 h. After removal of the solvent, the residue is chromatographed on silica gel using a mixture of petroleum ether/ethyl acetate in the ratio 2/1. Concentration of the corresponding eluate fractions furnish 10.37 g (89% of theory) of the title compound as an oil.

R_{f}=0.32 (petroleum ether/ethyl acetate=2/1)

### F2. (1RS,3RS,4RS)3-[3-(1,1-Difluoro-methoxy)-4-methoxy-phenyl]-4-nitrocyclohexanol

The title compound is prepared analogously as described in Example G1 starting from compound G2.

Starting from the starting compounds mentioned below, the following are obtained according to the procedure as in Example G1.

### F3. (1RS,3RS,4RS)-3-(3-Ethoxy-4-methoxy-phenyl)-4- nitrocyclohexanol

### F4. (1RS,3RS,4RS)-3-[4-(1,1-Difluoro-methoxy)-3-methoxy-phenyl]-4-nitrocyclohexanol

### F5. (1RS,3RS,4RS)-3-(3-(2,2-Difluoro-ethoxy)-4-methoxy-phenyl]-4-nitrocyclohexanol

### F6. [(1RS,6RS)-6-(3-Ethoxy-4-methoxy-phenyl)-cyclohex-3-enyl]carbamic acid tert-butyl ester

Starting from (1RS,6RS)-6-(3-ethoxy4-methoxy-phenyl)-cyclohex-3-enylamine (compound G6) the title compound is obtained analogously as described for compound F7.

EF: C20 H29 N O4; MW: 347.46, MS: 370.1 (Mna⁺)

### F7. [(1RS,6RS)-6-(3,4-Dimethoxy-phenyl)-clohex-3-enyl]carbamic acid tert-butyl ester

15.18 g (65.06 mmol) of (±)-cis-6-(3,4-dimethoxyphenyl)-cyclohex-3-enylamine (compound G7) and 14.21 g (65.11 mmol) of Boc₂O are stirred in dichloromethane for 2.5 h, then the solvent is removed and the residue crystallized from ethylacetate/n-heptane to give 19.1 g of the title compound.

EF: C19 H27 N O4; MW: 333.43, MS: 334.2 (MH⁺)

### G1. (1RS,3RS,4RS)3-(3,4-Dimethoxyphenyl)-4-nitrocyclohexanol

10 g of (1RS,3RS,4SR)-3-(3,4-dimethoxyphenyl)-4-nitrocyclohexanol (compound H1) are dissolved in 170 ml of absolute 1,2-dimethoxyethane. 14.3 ml of a 30% solution of sodium methanolate in methanol are added dropwise. After complete addition, stirring is continued for 10 min and a mixture consisting of 85 % phosphoric acid and methanol is added to pH 1. By adding of saturated potassium hydrogencarbonate solution the resulting suspension is neutralized. The mixture is diluted with water and dichloro methane, the organic layer is separated and extracted with dichloromethane. The solvents are removed under reduced pressure to yield the title compound as a pale yellow oil, which crystallizes. The title compound is used without further purification in the next step.

R_{f}=0.29 (petroleum ether/ethyl acetate=1/1) M.p.: 126-127° C.

### G2. (1RS,3RS,4SR)-3-[3-(1,1-Difluoro-methoxy)-4-methoxy-phenyl]-4-nitrocyclohexanol

The title compound is prepared analogously as described in Example H1 starting from compound H2.

Starting from the starting compounds mentioned below, the following are obtained according to the procedure as in Example H1.

### G3. (1RS,3RS,4SR)-3-(3-Ethoxy-4-methoxy-phenyl)-4-nitrocyclohexanol

### G4. (1RS,3RS,4SR)-3-[4-(1,1-Difluoro-methoxy)-3-methoxy-phenyl]-4-nitrocyclohexanol

### G5. (1RS,3RS,4SR)-3-(3-(2,2-Difluoro-ethoxy)-4-methoxy-phenyl]-4-nitrocyclohexanol

### G6. (1RS,6RS)-6-(3-Ethoxy-4-methoxy-phenyl)-cyclohex-3-enylamine

Starting from 2-ethoxy-1-methoxy-4-((1RS,6RS)-6-nitro-cyclohex-3-enyl)-benzene (compound H6) the title compound is obtained analogously as described for compound G7.

### G7. (±)-cis-6-(3,4-Dimethoxyphenyl)-cyclohex-3-enylamine

40 g of (±)-cis-1,2-dimethoxy-4-(2-nitrocyclohex-4-enyl)benzene (compound H7) are dissolved in 400 ml of ethanol and 40 g of zinc powder are added. After heating to boiling temperature, 65 ml of glacial acetic acid are added dropwise. Afterwards, the reaction mixture is filtrated and concentrated. The residue is redissolved in diluted hydrochloric acid and extraxted with toluene. The aqueous layer is alkalized using 6 N solution of sodium hydroxide and extracted several times with toluene. The combined organic phases of the alkalic extraction are dried using sodium sulfate and concentrated. The residue is chromatographed on silica gel. 11.5 g of the title compound are obtained.

### H1. (1RS,3RS,4SR)-3-(3,4-Dimethoxyphenyl)-4-nitrocyclohexanol

Under nitrogen atmosphere 16.76 g of (3RS,4SR)-3-(3,4-dimethoxyphenyl)-4-nitrocyclohexanone (compound I1) are dissolved in 300 ml of tetrahydrofurane, the solution is cooled to −78° C., and 75 ml of 1 M solution of potassium tri-sec-butylborohydride in tetrahydrofurane is added dropwise. After stirring for further 1 h, a mixture consisting of 30% hydrogeneperoxide solution and phosphate buffer solution is added. Stirring is continued for further 10 min, the reaction mixture is diluted with 400 ml of ethyl acetate and the aqueous layer is extracted with ethyl acetate, the combined organic phases are concentrated to give a foam, which is purified by chromatography on silica gel using a mixture of petroleum ether/ethyl acetate in the ratio 1/1 to furnish 10.18 g (60% of theory) of the title compound.

EF: C₁₄H₁₉NO₅; MW: 281.31 MS: 299.1 (MNH₄⁺) R_{f}=0.29 (petroleum ether/ethyl acetate=1/1) M.p.: 139-141° C.

### H2. (3RS,4SR)-3-[3-(1,1-Difluoro-methoxy)-4-methoxy-phenyl]-4-nitrocyclohexanone

The title compound is prepared analogously as described in Example I1 starting from compound I2.

Starting from the starting compounds mentioned below, the following are obtained according to the procedure as in Example I1.

### H3. (3RS,4SR)-3-(3-Ethoxy-4-methoxy-phenyl)-4-nitrocyclohexanone

### H4. (3RS,4SR)-3-[4-(1,1-Difluoro-methoxy)-3-methoxy-phenyl]-4-nitrocyclohexanone

### H5. (3RS,4SR)-3-(3-(2,2-Difluoro-ethoxy)-4-methoxy-phenyl]-4-nitrocyclohexanone

### H6. 2-Ethoxy-1-methoxy-4-((1RS,6RS)-6-nitro-cyclohex-3-enyl)-benzene

Starting from 2-ethoxy-1-methoxy-4-((1RS,6SR)-6-nitro-cyclohex-3-enyl)-benzene (compound I6) the title compound is obtained analogously as described for compound H7.

### H7. (±)-cis-1,2-Dimethoxy-4-(2-nitrocyclohex-4-enyl)benzene

10.0 g of (±)-trans-1,2-dimethoxy-4-(2-nitrocyclohex-4-enyl)benzene (compound 17) and 20.0 g of potassium hydroxide are dissolved in 150 ml of ethanol and 35 ml of dimethylformamide. A solution of 17.5 ml of conc. Sulfuric acid in 60 ml of ethanol is then added dropwise such that the internal temperature does not exceed 4° C. After stirring for 1 h, the mixture is added to 1 l of ice water, the precipitate is filtered off with suction, washed with water and dried, and the crude product is recrystallized in ethanol. 8.6 g of the title compound of m.p. 82.5-84° C. are obtained.

### I1. (3RS,4SR)-3-(3,4-Dimethoxyphenyl)-4-nitrocyclohexanone

90.0 g of 3,4-dimethoxy-ω-nitrostyrene (compound J1), 90 ml of 2-trimethylsilyloxy-1,3-butadiene and 180 ml of abs. toluene are put in an autoclave, where the mixture is stirred at 140° C. for 2 days and then cooled. After addition of 1000 ml of ethyl acetate, 300 ml of a 2 N solution of hydrochloric acid are dropped under stirring. The phases are separated and the aqueous layer is extracted three times with dichloromethane. The combined organic extracts are washed with saturated sodium hydrogencarbonate solution, dried over magnesium sulfate and the solvents are removed under reduced pressure to give 150 g of the crude title compound. Further purification is carried out by chromatography on silica gel using petroleum ether/ethyl acetate in the ratio 1/1 as eluent to give 81.5 g (67% of theory) of the pure title compound.

EF: C₁₄H₁₇NO₅; MW: 279.30 MS: 279 (M⁺), 297.1 (MNH₄⁺) R_{f}=0.47 (petroleum ether/ethyl acetate=1/1) M.p.: 147-148° C.

Starting from starting compounds, which are art-known or which can be obtained analogously to art-known compounds or according to art-known procedures, (such as e.g. as described in WO 95/01338 or analogously or similarly thereto) the following compounds are obtained according to the procedure as in Example J1:

### I2. 3-(1,1-Difluoro-methoxy)-4-methoxy-ω-nitrostyrene

### I3. 3-Ethoxy-4-methoxy-ω-nitrostyrene

### I4. 4-(1,1-Difluoro-methoxy)-3-methoxy-ω-nitrostyrene

### I5. 3-(2,2-Difluoro-ethoxy)4-methoxy-ω-nitrostyrene

The title compound is obtained starting from 3-(2,2-difluoro-ethoxy)-4-methoxy-benzaldehyde (compound K1) according to the procedure as in Example J1.

M.p.: 164-165° C.

### I6. 2-Ethoxy-1-methoxy-4-((1RS,6SR)-6-nitro-cyclohex-3-enyl)-benzene

Starting from 3-ethoxy-4-methoxy-ω-nitrostyrene (compound I3) the title compound is obtained analogously as described for compound I7.

### I7. (±)-trans-1,2-Dimethoxy-4-(2-nitrocyclohex-4-enyl)benzene

50.0 g of 3,4-dimethoxy-ω-nitrostyrene (compound J1), and 1.0 g (9.1 mmol) of hydroquinone are suspened in 200 ml of abs. Toluene and treated at −70° C. with 55.0 g (1.02 mol) of liquid 1,3-butadiene. The mixture is stirred at 160° C. for 6 days in an autoclave and then cooled. Some of the solvent is removed on a rotary evaporator, and the resulting precipitate is filtered off with suction and recrystallized in ethanol.

M.p.: 113.5-115.5° C.

### J1. 3,4-Dimethoxy-ω-nitrostyrene

207.0 g of 3,4-dimethoxybenzaldehyde,100.0 g of ammonium acetate and 125 ml of nitromethane are heated to boiling for 3-4 h in 1.0 l of glacial acetic acid. After cooling in an ice bath, the precipitate is filtered off with suction, rinsed with glacial acetic acid and petroleum ether and dried. M.p.: 140-141° C.

Yield: 179.0 g.

### K1.3-(2,2-Difluoro-ethoxy)-4-methoxy-benzaldehyde

10.04 g of isovanillin and 15.5 g of potassium carbonate are placed in an autoclave. 50 ml of DMF are added as well as 12.44 g of 2-bromo-1,1-difluoroethane. The autoclave is closed and heated at 60° C. for 20 h. Then the solids are filtered off and washed with 120 ml of DMF. About 120 ml of the solvent are distilled off and the residue poured on 200 ml of ice/water, where the product preciptates. After stirring the slurry for 30 minutes the product is filtered off and dried to give 13.69 g of the desired product.

M.p.: 66-68° C.

Commercial Utility

The compounds according to the invention have useful pharmacological properties which make them industrially utilizable. As selective cyclic nucleotide phosphodiesterase (PDE) inhibitors (specifically of type 4), they are suitable on the one hand as bronchial therapeutics (for the treatment of airway obstructions on account of their dilating action but also on account of their respiratory rate- or respiratory drive-increasing action) and for the removal of erectile dysfunction on account of their vascular dilating action, but on the other hand especially for the treatment of disorders, in particular of an inflammatory nature, e.g. of the airways (asthma prophylaxis), of the skin, of the intestine, of the eyes, of the CNS and of the joints, which are mediated by mediators such as histamine, PAF (platelet-activating factor), arachidonic acid derivatives such as leukotrienes and prostaglandins, cytokines, interleukins, chemokines, alpha-, beta- and gamma-interferon, tumor necrosis factor (TNF) or oxygen free radicals and proteases. In this context, the compounds according to the invention are distinguished by a low toxicity, a good enteral absorption (high bioavailability), a large therapeutic breadth and the absence of significant side effects.

On account of their PDE-inhibiting properties, the compounds according to the invention can be employed in human and veterinary medicine as therapeutics, where they can be used, for example, for the treatment and prophylaxis of the following illnesses: acute and chronic (in particular inflammatory and allergen-induced) airway disorders of varying origin (bronchitis, allergic bronchitis, bronchial asthma, emphysema, COPD); dermatoses (especially of proliferative, inflammatory and allergic type) such as psoriasis (vulgaris), toxic and allergic contact eczema, atopic eczema, seborrhoeic eczema, Lichen simplex, sunburn, pruritus in the anogenital area, alopecia areata, hypertrophic scars, discoid lupus erythematosus, follicular and widespread pyodermias, endogenous and exogenous acne, acne rosacea and other proliferative, inflammatory and allergic skin disorders; disorders which are based on an excessive release of TNF and leukotrienes, for example disorders of the arthritis type (rheumatoid arthritis, rheumatoid spondylitis, osteoarthritis and other arthritic conditions), disorders of the immune system (AIDS, multiple sclerosis), graft versus host reaction, allograft rejections, types of shock (septic shock, endotoxin shock, gramnegative sepsis, toxic shock syndrome and ARDS (adult respiratory distress syndrome)) and also generalized inflammations in the gastrointestinal region (Crohn's disease and ulcerative colitis); disorders which are based on allergic and/or chronic, immunological false reactions in the region of the upper airways (pharynx, nose) and the adjacent regions (paranasal sinuses, eyes), such as allergic rhinitis/sinusitis, chronic rhinitis/sinusitis, allergic conjunctivitis and also nasal polyps; but also disorders of the heart which can be treated by PDE inhibitors, such as cardiac insufficiency, or disorders which can be treated on account of the tissue-relaxant action of the PDE inhibitors, such as, for example, erectile dysfunction or colics of the kidneys and of the ureters in connection with kidney stones. In addition, the compounds of the invention are useful in the treatment of diabetes insipidus and conditions associated with cerebral metabolic inhibition, such as cerebral senility, senile dementia (Alzheimer's disease), memory impairment associated with Parkinson's disease or multiinfarct dementia; and also illnesses of the central nervous system, such as depressions or arteriosclerotic dementia; as well as for enhancing cognition. Yet in addition, the compounds of the invention are useful in the treatment of diabetes mellitus, leukaemia and osteoporosis.

The invention further relates to a method for the treatment of mammals, including humans, which are suffering from one of the above mentioned illnesses. The method is characterized in that a therapeutically active and pharmacologically effective and tolerable amount of one or more of the compounds according to the invention is administered to the ill mammal.

The invention further relates to the compounds according to the invention for use in the treatment and/or prophylaxis of illnesses, especially the illnesses mentioned.

The invention also relates to the use of the compounds according to the invention for the production of pharmaceutical compositions which are employed for the treatment and/or prophylaxis of the illnesses mentioned.

The invention also relates to the use of the compounds according to the invention for the production of pharmaceutical compositions for treating disorders which are mediated by phosphodiesterases, in particular PDE4-mediated disorders, such as, for example, those mentioned in the specification of this invention or those which are apparent or known to the skilled person.

The invention also relates to the use of the compounds according to the invention for the manufacture of pharmaceutical compositions having PDE4 inhibitory activity.

The invention furthermore relates to pharmaceutical compositions for the treatment and/or prophylaxis of the illnesses mentioned comprising one or more of the compounds according to the invention.

The invention yet furthermore relates to compositions comprising one or more compounds according to this invention and a pharmaceutically acceptable carrier. Said compositions can be used in therapy, such as e.g. for treating, preventing or ameliorating one or more of the abovementioned diseases.

The invention still yet furthermore relates to pharmaceutical compositions according to this invention having PDE, particularly PDE4, inhibitory activity.

Additionally, the invention relates to an article of manufacture, which comprises packaging material and a pharmaceutical agent contained within said packaging material, wherein the pharmaceutical agent is therapeutically effective for antagonizing the effects of the cyclic nucleotide phosphodiesterase of type 4 (PDE4), ameliorating the symptoms of an PDE4-mediated disorder, and wherein the packaging material comprises a label or package insert which indicates that the pharmaceutical agent is useful for preventing or treating PDE4-mediated disorders, and wherein said pharmaceutical agent comprises one or more compounds of formula 1 according to the invention. The packaging material, label and package insert otherwise parallel or resemble what is generally regarded as standard packaging material, labels and package inserts for pharmaceuticals having related utilities.

The pharmaceutical compositions are prepared by processes which are known per se and familiar to the person skilled in the art. As pharmaceutical compositions, the compounds according to the invention (=active compounds) are either employed as such, or preferably in combination with suitable pharmaceutical auxiliaries and/or excipients, e.g. in the form of tablets, coated tablets, capsules, caplets, suppositories, patches (e.g. as TTS), emulsions, suspensions, gels or solutions, the active compound content advantageously being between 0.1 and 95% and where, by the appropriate choice of the auxiliaries and/or excipients, a pharmaceutical administration form (e.g. a delayed release form or an enteric form) exactly suited to the active compound and/or to the desired onset of action can be achieved.

The person skilled in the art is familiar with auxiliaries, excipients, carriers, vehicles, diluents or adjuvants which are suitable for the desired pharmaceutical formulations on account of his/her expert knowledge. In addition to solvents, gel formers, ointment bases and other active compound excipients, for example antioxidants, dispersants, emulsifiers, preservatives, solubilizers, colorants, complexing agents or permeation promoters, can be used.

The administration of the pharmaceutical compositions according to the invention may be performed in any of the generally accepted modes of administration available in the art. Illustrative examples of suitable modes of administration include intravenous, oral, nasal, parenteral, topical, transdermal and rectal delivery. Oral delivery is preferred.

For the treatment of disorders of the respiratory tract, the compounds according to the invention are preferably also administered by inhalation in the form of an aerosol; the aerosol particles of solid, liquid or mixed composition preferably having a diameter of 0.5 to 10 μm, advantageously of 2 to 6 μm.

Aerosol generation can be carried out, for example, by pressure-driven jet atomizers or ultrasonic atomizers, but advantageously by propellant-driven metered aerosols or propellant-free administration of micronized active compounds from inhalation capsules.

Depending on the inhaler system used, in addition to the active compounds the administration forms additionally contain the required excipients, such as, for example, propellants (e.g. Frigen in the case of metered aerosols), surface-active substances, emulsifiers, stabilizers, preservatives, flavorings, fillers (e.g. lactose in the case of powder inhalers) or, if appropriate, further active compounds.

For the purposes of inhalation, a large number of apparatuses are available with which aerosols of optimum particle size can be generated and administered, using an inhalation technique which is as right as possible for the patient. In addition to the use of adaptors (spacers, expanders) and pear-shaped containers (e.g. Nebulator®, Volumatic®), and automatic devices emitting a puffer spray (Autohaler®), for metered aerosols, in particular in the case of powder inhalers, a number of technical solutions are available (e.g. Diskhaler®, Rotadisk®, Turbohaler® or the inhaler described in European Patent Application EP 0 505 321), using which an optimal administration of active compound can be achieved.

For the treatment of dermatoses, the compounds according to the invention are in particular administered in the form of those pharmaceutical compositions which are suitable for topical application. For the production of the pharmaceutical compositions, the compounds according to the invention (=active compounds) are preferably mixed with suitable pharmaceutical auxiliaries and further processed to give suitable pharmaceutical formulations. Suitable pharmaceutical formulations are, for example, powders, emulsions, suspensions, sprays, oils, ointments, fatty ointments, creams, pastes, gels or solutions.

The pharmaceutical compositions according to the invention are prepared by processes known per se. The dosage of the active compounds is carried out in the order of magnitude customary for PDE inhibitors. Topical application forms (such as ointments) for the treatment of dermatoses thus contain the active compounds in a concentration of, for example, 0.1-99%. The dose for administration by inhalation is customarly between 0.01 and 3 mg per day. The customary dose in the case of systemic therapy (p.o. or i.v.) is between 0.003 and 3 mg/kg per day. In another embodiment, the dose for administration by inhalation is between 0.1 and 3 mg per day, and the dose in the case of systemic therapy (p.o. or i.v.) is between 0.03 and 3 mg/kg per day.

Biological Investigations

The second messenger cyclic AMP (cAMP) is well-known for inhibiting inflammatory and immunocompetent cells. The PDE4 isoenzyme is broadly expressed in cells involved in the initiation and propagation of inflammatory diseases (H Tenor and C Schudt, in “Phosphodiesterase Inhibitors”, 21-40, “The Handbook of Immunopharmacology”, Academic Press, 1996), and its inhibition leads to an increase of the intracellular cAMP concentration and thus to the inhibition of cellular activation (J E Souness et al., Immunopharmacology 47: 127-162, 2000).

The antiinflammatory potential of PDE4 inhibitors in vivo in various animal models has been described (M M Teixeira, TiPS 18: 164-170, 1997). For the investigation of PDE4 inhibition on the cellular level (in vitro), a large variety of proinflammatory responses can be measured. Examples are the superoxide production of neutrophilic (C Schudt et al., Arch Pharmacol 344: 682-690, 1991) or eosinophilic (A Hatzelmann et al., Brit J Pharmacol 114: 821-831, 1995) granulocytes, which can be measured as luminolenhanced chemiluminescence, or the synthesis of tumor necrosis factor-α in monocytes, macrophages or dendritic cells (Gantner et al., Brit J Pharmacol 121: 221-231, 1997, and Pulmonary Pharmacol Therap 12: 377-386, 1999). In addition, the immunomodulatory potential of PDE4 inhibitors is evident from the inhibition of T-cell responses like cytokine synthesis or proliferation (D M Essayan, Biochem Pharmacol 57: 965-973, 1999). Substances which inhibit the secretion of the afore-mentioned proinflammatory mediators are those which inhibit PDE4. PDE4 inhibition by the compounds according to the invention is thus a central indicator for the suppression of inflammatory processes.

Methods for Measuring Inhibition of PDE4 Activity

The PDE4B2 (GB no. M97515) was a gift of Prof. M. Conti (Stanford University, USA). It was amplified from the original plasmid (pCMV5) via PCR with primers Rb9 (5′-GCCAGCGTGCAAATAATGAAGG-3′) and Rb10 (5′-AGAGGGGGATTATGTATCCAC-3′) and cloned into the pCR-Bac vector (Invitrogen, Groningen, NL).

The recombinant baculovirus was prepared by means of homologous recombination in SF9 insect cells.

The expression plasmid was cotransfected with Bac-N-Blue (Invitrogen, Groningen, NL) or Baculo-Gold DNA (Pharmingen, Hamburg) using a standard protocol (Pharmingen, Hamburg). Wt virus-free recombinant virus supernatant was selected using plaque assay methods. After that, high-titre virus supernatant was prepared by amplifying 3 times. PDE was expressed in SF21 cells by infecting 2×10⁶ cells/ml with an MOI (multiplicity of infection) between 1 and 10 in serum-free SF900 medium (Life Technologies, Paisley, UK). The cells were cultured at 28° C. for 48-72 hours, after which they were pelleted for 5-10 min at 1000 g and 4° C.

The SF21 insect cells were resuspended, at a concentration of approx. 10⁷ cells/ml, in ice-cold (4° C.) homogenization buffer (20 mM Tris, pH 8.2, containing the following additions: 140 mM NaCl, 3.8 mM KCl, 1 mM EGTA, 1 mM MgCl₂, 10 mM β-mercaptoethanol, 2 mM benzamidine, 0.4 mM Pefablock, 10 μM leupeptin, 10 μM pepstatin A, 5 μM trypsin inhibitor) and disrupted by ultrasonication. The homogenate was then centrifuged for 10 min at 1000×g and the supernatant was stored at −80° C. until subsequent use (see below). The protein content was determined by the Bradford method (BioRad, Munich) using BSA as the standard.

PDE4B2 activity is inhibited by the said compounds in a modified SPA (scintillation proximity assay) test, supplied by Amersham Biosciences (see procedural instructions “phosphodiesterase [3H]cAMP SPA enzyme assay, code TRKQ 7090”), carried out in 96-well microtitre plates (MTP's). The test volume is 100 μl and contains 20 mM Tris buffer (pH 7.4), 0.1 mg of BSA (bovine serum albumin)/ml, 5 mM Mg²⁺, 0.5 μM cAMP (including about 50,000 cpm of [3H]cAMP), 1 μl of the respective substance dilution in DMSO and sufficient recombinant PDE (1000×g supernatant, see above) to ensure that 10-20% of the cAMP is converted under the said experimental conditions. The final concentration of DMSO in the assay (1% v/v) does not substantially affect the activity of the PDE investigated. After a preincubation of 5 min at 37° C., the reaction is started by adding the substrate (cAMP) and the assay is incubated for a further 15 min; after that, it is stopped by adding SPA beads (50 μl). In accordance with the manufacturer's instructions, the SPA beads had previously been resuspended in water, but were then diluted 1:3 (v/v) in water; the diluted solution also contains 3 mM IBMX to ensure a complete PDE activity stop. After the beads have been sedimented (>30 min), the MTP's are analyzed in commercially available luminescence detection devices. The corresponding IC₅₀ values of the compounds for the inhibition of PDE activity are determined from the concentration-effect curves by means of non-linear regression.

Representative inhibitory values determined for the compounds according to the invention follow from the following table A, in which the numbers of the compounds correspond to the numbers of the Examples.

TABLE A
Inhibition of the PDE4 activity

| Compound | −log IC₅₀ |
|---|---|
| 1 to 35 | The inhibitory values |
| | of these listed com- |
| | pounds 1 to 35 are in |
| | the range from 8.06 to |
| | 9.02 |
| 66, 68, 69, and | The inhibitory values |
| 71 to 75 | of these listed com- |
| | pounds 66, 68, 69, |
| | and 71 to 75 are in the |
| | range from 6.42 to |
| | 8.75 |

1. A compound of formula I, 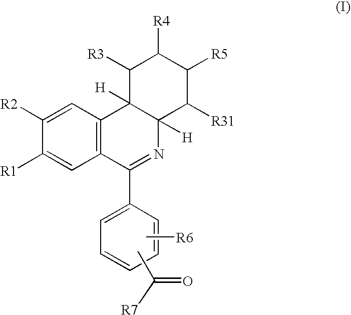
in which
R1 is hydroxyl, 1-4C-alkoxy, 3-7C-cycloalkoxy, 3-7C-cycloalkylmethoxy, 2,2-difluoroethoxy, or completely or predominantly fluorine-substituted 1-4C-alkoxy,
R2 is hydroxyl, 1-4C-alkoxy, 3-7C-cycloalkoxy, 3-7C-cycloalkylmethoxy, 2,2-difluoroethoxy, or completely or predominantly fluorine-substituted 1-4C-alkoxy,
or in which
R1 and R2 together are a 1-2C-alkylenedioxy group,
R3 is hydrogen or 1-4C-alkyl,
R31 is hydrogen or 1-4C-alkyl,
either, in a first embodiment (embodiment a),
R4 is —O—R41, in which
R41 is hydrogen, 1-4C-alkyl, 1-4C-alkoxy-1-4C-alkyl, hydroxy-2-4C-alkyl, 1-7C-alkylcarbonyl, or completely or predominantly fluorine-substituted 1-4C-alkyl, and
R5 is hydrogen or 1-4C-alkyl,
or, in a second embodiment (embodiment b),
R4 is hydrogen or 1-4C-alkyl, and
R5 is —O—R51, in which
R51 is hydrogen, 1-4C-alkyl, 1-4C-alkoxy-1-4C-alkyl, hydroxy-2-4C-alkyl, 1-7C-alkylcarbonyl, or completely or predominantly fluorine-substituted 1-4C-alkyl,
R6 is hydrogen, halogen, 1-4C-alkyl or 1-4C-alkoxy,
either,
in a first aspect (aspect 1),
R7 is —N(R8)R9, in which
R8 is hydrogen, 1-4C-alkyl or 1-4C-alkoxy-2-4C-alkyl,
R9 is hydrogen, 1-4C-alkyl, mono- or di-1-4C-alkoxy-2-4C-alkyl, hydroxy-2-4C-alkyl, mono- or di-1-4C-alkoxycarbonyl-1-4C-alkyl, Har1, pyridinyl-1-4C-alkyl, 3-7C-cycloalkyl, or 2-4C-alkyl substituted by —NR(93)R94, in which
Har1 is optionally substituted by R91 and/or R92, and is a 5- to 10-membered monocyclic or fused bicyclic unsaturated heteroaryl radical comprising 1 to 4 heteroatoms independently selected from the group consisting of oxygen, nitrogen and sulfur, in which
R91 is 1-4C-alkyl or 1-4C-alkoxy,
R92 is 1-4C-alkyl or 1-4C-alkoxy,
R93 is hydrogen or 1-4C-alkyl,
R94 is hydrogen or 1-4C-alkyl,
or R93 and R94 together and with inclusion of the nitrogen atom, to which they are attached, form a heterocyclic ring Het1, in which
Het1 is optionally substituted by R931, and is a 3- to 7-membered saturated monocyclic heterocyclic ring radical comprising the nitrogen atom, to which R93 and R94 are bonded, and optionally one further heteroatom selected from the group consisting of oxygen, nitrogen and sulfur, in which
R931 is 1-4C-alkyl,
or R8 and R9 together and with inclusion of the nitrogen atom, to which they are attached, form a heterocyclic ring Het2, in which
Het2 is optionally substituted by R10, and is a 3- to 7-membered saturated monocyclic heterocyclic ring radical comprising the nitrogen atom, to which R8 and R9 are bonded, and optionally one further heteroatom selected from the group consisting of oxygen, nitrogen and sulfur, in which
R10 is 1-4C-alkyl, —C(O)R11, pyridyl, 2-4C-alkyl substituted by —NR(14)R15, or 1-4C-alkyl substituted by —C(O)N(R16)R17, in which
R11 is 1-4C-alkyl substituted by —NR(12)R13, in which
R12 is hydrogen or 1-4C-alkyl,
R13 is hydrogen or 1-4C-alkyl,
or R12 and R13 together and with inclusion of the nitrogen atom, to which they are attached, form a heterocyclic ring Het3, in which
Het3 is optionally substituted by R121, and is a 3- to 7-membered saturated monocyclic heterocyclic ring radical comprising the nitrogen atom, to which R12 and R13 are bonded, and optionally one further heteroatom selected from the group consisting of oxygen, nitrogen and sulfur, in which
R121 is 1-4C-alkyl,
R14 is hydrogen or 1-4C-alkyl,
R15 is hydrogen or 1-4C-alkyl,
or R14 and R15 together and with inclusion of the nitrogen atom, to which they are attached, form a heterocyclic ring Het4, in which
Het4 is optionally substituted by R141, and is a 3- to 7-membered saturated monocyclic heterocyclic ring radical comprising the nitrogen atom, to which R14 and R15 are bonded, and optionally one further heteroatom selected from the group consisting of oxygen, nitrogen and sulfur, in which
R141 is 1-4C-alkyl,
R16 is hydrogen, 1-4C-alkyl or pyridyl,
R17 is hydrogen or 1-4C-alkyl,
or R16 and R17 together and with inclusion of the nitrogen atom, to which they are attached, form a heterocyclic ring Het5, in which
Het5 is optionally substituted by R161, and is a 3- to 7-membered saturated monocyclic heterocyclic ring radical comprising the nitrogen atom, to which R16 and R17 are bonded, and optionally one further heteroatom selected from the group consisting of oxygen, nitrogen and sulfur, in which
R141 is 1-4C-alkyl,
or,
in a second aspect (aspect 2),
R7 is —NH—N(R18)R19, in which
R18 is hydrogen,
R19 is —C(O)R20, or R21-substituted phenyl, in which
R20 is Har2, Het6, or Aryl-1-4C-alkyl, in which
Har2 is optionally substituted by R201 and/or R202, and is a 5- to 10-membered monocyclic or fused bicyclic unsaturated heteroaryl radical comprising 1 to 4 heteroatoms independently selected from the group consisting of oxygen, nitrogen and sulfur, in which
R201 is 1-4C-alkyl or 1-4C-alkoxy,
R202 is 1-4C-alkyl or 1-4C-alkoxy,
Het6 is optionally substituted by R203 and/or R204, and is a monocyclic 3- to 7-membered saturated heterocyclic ring radical comprising one to three heteroatoms, each of which is selected from the group consisting of nitrogen, oxygen and sulfur, in which
R203 is 1-4C-alkyl,
R204 is 1-4C-alkyl,
Aryl is R205- and/or R206-substituted phenyl,
R205 is 1-4C-alkoxy,
R206 is 1-4C-alkoxy,
R21 is aminosulphonyl,
or R18 and R19 together and with inclusion of the nitrogen atom, to which they are attached, form a heterocyclic ring Het7, in which
Het7 is optionally substituted by R181, and is a 3- to 7-membered saturated monocyclic heterocyclic ring radical comprising the nitrogen atom, to which R18 and R19 are bonded, and optionally one further heteroatom selected from the group consisting of oxygen, nitrogen and sulfur, in which
R181 is 1-4C-alkyl,
or a salt, enantiomer, N-oxide, salt of an N-oxide or enantiomer thereof.

2. A compound of formula I according to claim 1 in which
R1 is 1-2C-alkoxy, 3-5C-cycloalkoxy, 3-5C-cycloalkylmethoxy, 2,2-difluoroethoxy, or completely or predominantly fluorine-substituted 1-2C-alkoxy,
R2 is 1-2C-alkoxy, 3-5C-cycloalkoxy, 3-5C-cycloalkylmethoxy, 2,2-difluoroethoxy, or completely or predominantly fluorine-substituted 1-2C-alkoxy,
R3 is hydrogen,
R31 is hydrogen,
either, in a first embodiment (embodiment a),
R4 is —O—R41, in which
R41 is hydrogen or 1-4C-alkylcarbonyl, and
R5 is hydrogen,
or, in a second embodiment (embodiment b),
R4 is hydrogen, and
R5 is —O—R51, in which
R51 is hydrogen or 1-4C-alkylcarbonyl,
R6 is hydrogen,
either,
in a first aspect (aspect 1),
R7 is —N(R8)R9, in which
R8 is hydrogen, 1-4C-alkyl or 1-4C-alkoxy-2-4C-alkyl,
R9 is hydrogen, 1-4C-alkyl, mono- or di-1-4C-alkoxy-2-4C-alkyl, hydroxy-2-4C-alkyl, mono- or di-1-4C-alkoxycarbonyl-1-4C-alkyl, Har1, pyridinyl-1-4C-alkyl, 3-7C-cycloalkyl, or 2-4C-alkyl substituted by —NR(93)R94, in which
Har1 is optionally substituted by R91 and/or R92, and is a 5- to 10-membered monocyclic or fused bicyclic unsaturated heteroaryl radical comprising 1 to 4 heteroatoms independently selected from the group consisting of oxygen, nitrogen and sulfur, in which
R91 is 1-4C-alkyl or 1-4C-alkoxy,
R92 is 1-4C-alkyl or 1-4C-alkoxy,
R93 is hydrogen or 1-4C-alkyl,
R94 is hydrogen or 1-4C-alkyl,
or R93 and R94 together and with inclusion of the nitrogen atom, to which they are attached, form a heterocyclic ring Het1, in which
Het1 is optionally substituted by R931, and is a 3- to 7-membered saturated monocyclic heterocyclic ring radical comprising the nitrogen atom, to which R93 and R94 are bonded, and optionally one further heteroatom selected from the group consisting of oxygen, nitrogen and sulfur, in which
R931 is 1-4C-alkyl,
or R8 and R9 together and with inclusion of the nitrogen atom, to which they are attached, form a heterocyclic ring Het2, in which
Het2 is optionally substituted by R10, and is a 3- to 7-membered saturated monocyclic heterocyclic ring radical comprising the nitrogen atom, to which R8 and R9 are bonded, and optionally one further heteroatom selected from the group consisting of oxygen, nitrogen and sulfur, in which
R10 is 1-4C-alkyl, —C(O)R11, pyridyl, 2-4C-alkyl substituted by —NR(14)R15, or 1-4C-alkyl substituted by —C(O)N(R16)R17, in which
R11is 1-4C-alkyl substituted by —NR(12)R13, in which
R12 is hydrogen or 1-4C-alkyl,
R13 is hydrogen or 1-4C-alkyl,
or R12 and R13 together and with inclusion of the nitrogen atom, to which they are attached, form a heterocyclic ring Het3, in which
Het3 is optionally substituted by R121, and is a 3- to 7-membered saturated monocyclic heterocyclic ring radical comprising the nitrogen atom, to which R12 and R13 are bonded, and optionally one further heteroatom selected from the group consisting of oxygen, nitrogen and sulfur, in which
R121 is 1-4C-alkyl,
R14 is hydrogen or 1-4C-alkyl,
R15 is hydrogen or 1-4C-alkyl,
or R14 and R15 together and with inclusion of the nitrogen atom, to which they are attached, form a heterocyclic ring Het4, in which
Het4 is optionally substituted by R141, and is a 3- to 7-membered saturated monocyclic heterocyclic ring radical comprising the nitrogen atom, to which R14 and R15 are bonded, and optionally one further heteroatom selected from the group consisting of oxygen, nitrogen and sulfur, in which
R141 is 1-4C-alkyl,
R16 is hydrogen, 1-4C-alkyl or pyridyl,
R17 is hydrogen or 1-4C-alkyl,
or R16 and R17 together and with inclusion of the nitrogen atom, to which they are attached, form a heterocyclic ring Het5, in which
Het5 is optionally substituted by R161, and is a 3- to 7-membered saturated monocyclic heterocyclic ring radical comprising the nitrogen atom, to which R16 and R17 are bonded, and optionally one further heteroatom selected from the group consisting of oxygen, nitrogen and sulfur, in which
R141 is 1-4C-alkyl,
or,
in a second aspect (aspect 2),
R7 is —NH—N(R18)R19, in which
R18 is hydrogen,
R19 is —C(O)R20, or R21-substituted phenyl, in which
R20 is Har2, Het6, or Aryl-1-4C-alkyl, in which
Har2 is optionally substituted by R201 and/or R202, and is a 5- to 10-membered monocyclic or fused bicyclic unsaturated heteroaryl radical comprising 1 to 4 heteroatoms independently selected from the group consisting of oxygen, nitrogen and sulfur, in which
R201 is 1-4C-alkyl or 1-4C-alkoxy,
R202 is 1-4C-alkyl or 1-4C-alkoxy,
Het6 is optionally substituted by R203 and/or R204, and is a monocyclic 3- to 7-membered saturated heterocyclic ring radical comprising one to three heteroatoms, each of which is selected from the group consisting of nitrogen, oxygen and sulfur, in which
R203 is 1-4C-alkyl,
R204 is 1-4C-alkyl,
Aryl is R205- and/or R206-substituted phenyl,
R205 is 1-4C-alkoxy,
R206 is 1-4C-alkoxy,
R21 is aminosulphonyl,
or R18 and R19 together and with inclusion of the nitrogen atom, to which they are attached, form a heterocyclic ring Het7, in which
Het7 is optionally substituted by R181, and is a 3- to 7-membered saturated monocyclic heterocyclic ring radical comprising the nitrogen atom, to which R18 and R19 are bonded, and optionally one further heteroatom selected from the group consisting of oxygen, nitrogen and sulfur, in which
R181 is 1-4C-alkyl,
or a salt, enantiomer, N-oxide, salt of an N-oxide or enantiomer thereof.

3. A compound of formula I according to claim 1 in which
R1 is 1-2C-alkoxy, 2,2-difluoroethoxy, or completely or predominantly fluorine-substituted 1-2C-alkoxy,
R2 is 1-2C-alkoxy, 2,2-difluoroethoxy, or completely or predominantly fluorine-substituted 1-2C-alkoxy,
R3 is hydrogen,
R31 is hydrogen,
R4 is —O—R41, in which
R41 is hydrogen or 1-4C-alkylcarbonyl,
R5 is hydrogen,
R6 is hydrogen,
either,
in a first aspect (aspect 1),
R7 is —N(R8)R9, in which
R8 is hydrogen, 1-4C-alkyl or 1-4C-alkoxy-2-4C-alkyl,
R9 is hydrogen, 1-4C-alkyl, mono- or di-1-4C-alkoxy-2-4C-alkyl, hydroxy-2-4C-alkyl, mono- or di-1-4C-alkoxycarbonyl-1-4C-alkyl, Har1, pyridinyl-1-4C-alkyl, 3-7C-cycloalkyl, or 2-4C-alkyl substituted by —NR(93)R94, in which
either
Har1 is optionally substituted by R91 and/or R92, and is a 9- or 10-membered fused bicyclic unsaturated heteroaryl radical comprising 1 to 4 heteroatoms independently selected from the group consisting of oxygen, nitrogen and sulfur, in which
R91 is 1-4C-alkyl,
R92 is 1-4C-alkyl,
or
Har1 is optionally substituted by R91 and/or R92, and is a 6-membered monocyclic unsaturated heteroaryl radical comprising one or two nitrogen atoms, in which
R91 is 1-4C-alkoxy,
R92 is 1-4C-alkoxy,
R93 is hydrogen or 1-4C-alkyl,
R94 is hydrogen or 1-4C-alkyl,
or R93 and R94 together and with inclusion of the nitrogen atom, to which they are attached, form a heterocyclic ring Het1, in which
Het1 is optionally substituted by R931, and is a 3- to 7-membered saturated monocyclic heterocyclic ring radical comprising the nitrogen atom, to which R93 and R94 are bonded, and optionally one further heteroatom selected from the group consisting of oxygen, nitrogen and sulfur, in which
R931 is 1-4C-alkyl,
or R8 and R9 together and with inclusion of the nitrogen atom, to which they are attached, form a heterocyclic ring Het2, in which
Het2 is optionally substituted by R10, and is a 3- to 7-membered saturated monocyclic heterocyclic ring radical comprising the nitrogen atom, to which R8 and R9 are bonded, and optionally one further heteroatom selected from the group consisting of oxygen, nitrogen and sulfur, in which
R10 is 1-4C-alkyl, —C(O)R11, pyridyl, 2-4C-alkyl substituted by —NR(14)R15, or 1-4C-alkyl substituted by —C(O)N(R16)R17, in which
R11is 1-4C-alkyl substituted by —NR(12)R13, in which
R12 is hydrogen or 1-4C-alkyl,
R13 is hydrogen or 1-4C-alkyl,
or R12 and R13 together and with inclusion of the nitrogen atom, to which they are attached, form a heterocyclic ring Het3, in which
Het3 is optionally substituted by R121, and is a 3- to 7-membered saturated monocyclic heterocyclic ring radical comprising the nitrogen atom, to which R12 and R13 are bonded, and optionally one further heteroatom selected from the group consisting of oxygen, nitrogen and sulfur, in which
R121 is 1-4C-alkyl,
R14 is hydrogen or 1-4C-alkyl,
R15 is hydrogen or 1-4C-alkyl,
or R14 and R15 together and with inclusion of the nitrogen atom, to which they are attached, form a heterocyclic ring Het4, in which
Het4 is optionally substituted by R141, and is a 3- to 7-membered saturated monocyclic heterocyclic ring radical comprising the nitrogen atom, to which R14 and R15 are bonded, and optionally one further heteroatom selected from the group consisting of oxygen, nitrogen and sulfur, in which
R141 is 1-4C-alkyl,
R16 is hydrogen, 1-4C-alkyl or pyridyl,
R17 is hydrogen or 1-4C-alkyl,
or R16 and R17 together and with inclusion of the nitrogen atom, to which they are attached, form a heterocyclic ring Het5, in which
Het5 is optionally substituted by R161, and is a 3- to 7-membered saturated monocyclic heterocyclic ring radical comprising the nitrogen atom, to which R16 and R17 are bonded, and optionally one further heteroatom selected from the group consisting of oxygen, nitrogen and sulfur, in which
R141 is 1-4C-alkyl,
or,
in a second aspect (aspect 2),
R7 is —NH—N(R18)R19, in which
R18 is hydrogen,
R19 is —C(O)R20, or R21-substituted phenyl, in which
R20 is Har2, Het6, or Aryl-1-4C-alkyl, in which
Har2 is a 6-membered monocyclic unsaturated heteroaryl radical comprising one or two nitrogen atoms,
Het6 is optionally substituted by R203 and/or R204, and is a monocyclic 3- to 7-membered saturated heterocyclic ring radical comprising one to three heteroatoms, each of which is selected from the group consisting of nitrogen, oxygen and sulfur, in which
R203 is 1-4C-alkyl,
R204 is 1-4C-alkyl,
Aryl is R205- and/or R206-substituted phenyl,
R205 is 1-4C-alkoxy,
R206 is 1-4C-alkoxy,
R21 is aminosulphonyl,
or R18 and R19 together and with inclusion of the nitrogen atom, to which they are attached, form a heterocyclic ring Het7, in which
Het7 is optionally substituted by R181, and is a 3- to 7-membered saturated monocyclic heterocyclic ring radical comprising the nitrogen atom, to which R18 and R19 are bonded, and optionally one further heteroatom selected from the group consisting of oxygen, nitrogen and sulfur, in which
R181 is 1-4C-alkyl,
or a salt, enantiomer, N-oxide, salt of an N-oxide or enantiomer thereof.

4. A compound of formula I according to claim 1 in which
R1 is 1-2C-alkoxy, 2,2-difluoroethoxy, or completely or predominantly fluorine-substituted 1-2C-alkoxy,
R2 is 1-2C-alkoxy, 2,2-difluoroethoxy, or completely or predominantly fluorine-substituted 1-2C-alkoxy,
R3 is hydrogen,
R31 is hydrogen,
R4 is —O—R41, in which
R41 is hydrogen,
R5 is hydrogen,
R6 is hydrogen,
either,
in a first aspect (aspect 1),
R7 is —N(R8)R9, in which
R8 is hydrogen, 1-4C-alkyl or 1-4C-alkoxy-2-4C-alkyl,
R9 is 1-4C-alkyl, mono- or di-1-4C-alkoxy-2-4C-alkyl, hydroxy-2-4C-alkyl, mono- or di-1-2C-alkoxycarbonyl-1-4C-alkyl, Har1, pyridinyl-1-4C-alkyl, 3-5C-cycloalkyl, or 2-4C-alkyl substituted by —NR(93)R94, in which
Har1 is 2,6-dimethoxypyridinyl, quinolinyl, 2,3-dimethyl-imidazo[1,2-a]pyridinyl or [1,7]naphthyridinyl,
R93 and R94 together and with inclusion of the nitrogen atom, to which they are attached, form a heterocyclic ring Het1, in which
Het1 is morpholinyl,
or R8 and R9 together and with inclusion of the nitrogen atom, to which they are attached, form a heterocyclic ring Het2, in which
Het2 is pyrrolidinyl, morpholinyl or 4N—(R10)-piperazinyl, in which
R10 is —C(O)R11, pyridyl, 2-4C-alkyl substituted by —NR(14)R15, or 1-4C-alkyl substituted by C(O)N(R16)R17, in which
R11is 1-4C-alkyl substituted by —NR(12)R13, in which
R12 is 1-4C-alkyl,
R13 is 1-4C-alkyl,
or R12 and R13 together and with inclusion of the nitrogen atom, to which they are attached, form a heterocyclic ring Het3, in which
Het3 is morpholinyl,
R14 is 1-4C-alkyl,
R15 is 1-4C-alkyl,
or R14 and R15 together and with inclusion of the nitrogen atom, to which they are attached, form a heterocyclic ring Het4, in which
Het4 is morpholinyl,
R16 is 1-4C-alkyl or pyridyl,
R17 is hydrogen or 1-4C-alkyl,
or R16 and R17 together and with inclusion of the nitrogen atom, to which they are attached, form a heterocyclic ring Het5, in which
Het5 is pyrrolidinyl or morpholinyl,
or,
in a second aspect (aspect 2),
R7 is —NH—N(R18)R19, in which
R18 is hydrogen,
R19 is —C(O)R20, or R21-substituted phenyl, in which
R20 is pyridinyl, morpholinyl, 1N—(R203)-4N—(R204)-piperazinyl, or Aryl-1-2C-alkyl, in which
R203 is 1-4C-alkyl,
R204 is 1-4C-alkyl,
Aryl is 3,4-dimethoxyphenyl,
R21 is aminosulphonyl,
or R18 and R19 together and with inclusion of the nitrogen atom, to which they are attached, form a heterocyclic ring Het7, in which
Het7 is morpholinyl or 4N—(R181)-piperazinyl, in which
R181 is 1-4C-alkyl,
or a salt, enantiomer, N-oxide, salt of an N-oxide or enantiomer thereof.

5. A compound of formula I according to claim 1 in which
R1 is 1-2C-alkoxy, 2,2-difluoroethoxy, or completely or predominantly fluorine-substituted 1-2C-alkoxy,
R2 is 1-2C-alkoxy, 2,2-difluoroethoxy, or completely or predominantly fluorine-substituted 1-2C-alkoxy,
R3 is hydrogen,
R31 is hydrogen,
R4 is —O—R41, in which
R41 is hydrogen,
R5 is hydrogen,
R6 is hydrogen,
either,
in a first aspect (aspect 1),
R7 is —N(R8)R9, in which
R8 is hydrogen, methyl, ethyl or 2-methoxyethyl,
R9 is methyl, 2-methoxyethyl, methoxycarbonylmethyl, 1,2-di-(methoxycarbonyl)-ethyl, Har1, 2-pyridinyl-ethyl, cyclopropyl, or 2-3C-alkyl substituted by —NR(93)R94, in which
Har1 is 2,6-dimethoxypyridinyl, quinolinyl, 2,3-dimethyl-imidazo[1,2-a]pyridinyl or [1,7]naphthyridinyl,
R93 and R94 together and with inclusion of the nitrogen atom, to which they are attached, form a heterocyclic ring Het1, in which
Het1 is morpholinyl,
or R8 and R9 together and with inclusion of the nitrogen atom, to which they are attached, form a heterocyclic ring Het2, in which
Het2 is pyrrolidinyl, morpholinyl or 4N—(R10)-piperazinyl, in which
R10 is pyridyl, ethyl substituted by —NR(14)R15, or methyl substituted by —C(O)N(R16)R17, in which
R14 is methyl,
R15 is methyl,
or R14 and R15 together and with inclusion of the nitrogen atom, to which they are attached, form a heterocyclic ring Het4, in which
Het4 is morpholinyl,
R16 is methyl or pyridyl,
R17 is hydrogen or methyl,
or R16 and R17 together and with inclusion of the nitrogen atom, to which they are attached, form a heterocyclic ring Het5, in which
Het5 is pyrrolidinyl or morpholinyl,
or,
in a second aspect (aspect 2),
R7 is —NH—N(R18)R19, in which
R18 is hydrogen,
R19 is —C(O)R20, or R21-substituted phenyl, in which
R20 is pyridinyl, or morpholin-4-yl,
R21 is aminosulphonyl,
or R18 and R19 together and with inclusion of the nitrogen atom, to which they are attached, form a heterocyclic ring Het7, in which
Het7 is morpholinyl or 4N—(R181)-piperazinyl, in which
R181 is methyl,
or a salt, enantiomer, N-oxide, salt of an N-oxide or enantiomer thereof.

6. A compound of formula I according to claim 1 comprising one or more of the following:
one of R1 and R2 is methoxy, and the other is methoxy, ethoxy, difluoromethoxy or 2,2-difluoroethoxy, and
R3 and R31 are both hydrogen,
R4 is —O—R41, in which
R41 is hydrogen, or 1-4C-alkylcarbonyl and
R5 is hydrogen and
R6 is hydrogen,
or a salt, enantiomer, N-oxide, salt of an N-oxide or enantiomer thereof.

7. A compound of formula I according to claim 1 comprising one or more of the following:
R1 is methoxy,
R2 is methoxy, ethoxy, difluoromethoxy or 2,2-difluoroethoxy, and
R3 and R31 are both hydrogen,
R4 is —O—R41, in which
R41 is hydrogen, and
R5 is hydrogen,
R6 is hydrogen, and —C(O)R7 is attached in the meta or para position with respect to the binding position in which the phenyl moiety is bonded,
or a salt, enantiomer, N-oxide, salt of an N-oxide or enantiomer thereof.

8. A compound of formula I according to claim 1 in which
R1 is methoxy,
R2 is methoxy, ethoxy, 2,2-difluoroethoxy, or difluoromethoxy,
R3 is hydrogen,
R31 is hydrogen,
R4 is —O—R41, in which
R41 is hydrogen,
R5 is hydrogen,
R6 is hydrogen,
R7 is —N(R8)R9, in which
R8 is hydrogen, methyl, ethyl, or isopropyl,
R9 is methyl, ethyl, isopropyl, cyclopropyl or cyclobutyl,
whereby the radical —C(O)R7 is attached in the meta or para position with respect to the binding position in which the phenyl moiety is bonded
or a salt, enantiomer, N-oxide, salt of an N-oxide or enantiomer thereof.

9. A compound of formula I according to claim 1 in which
R1 is methoxy,
R2 is ethoxy, 2,2-difluoroethoxy, or difluoromethoxy,
R3 is hydrogen,
R31 is hydrogen,
R4 is —O—R41, in which
R41 is hydrogen,
R5 is hydrogen,
R6 is hydrogen,
R7 is —N(R8)R9, in which
either
R8 is isopropyl, and
R9 is isopropyl,
or
R8 is hydrogen, and
R9 is cyclopropyl or cyclobutyl,
whereby the radical —C(O)R7 is attached in the meta or para position with respect to the binding position in which the phenyl moiety is bonded,
or a salt, enantiomer, N-oxide, salt of an N-oxide or enantiomer thereof.

10. A compound of formula I according to claim 1 selected from the group consisting of
4-((2RS,4aRS,10bRS)-2-Hydroxy-8,9-dimethoxy-1,2,3,4,4a,10b-hexahydro-phenanthridin-6-yl)-N-(2-morpholin-4-yl-ethyl)-benzamide,
4-((2RS,4aRS,10bRS)-2-Hydroxy-8,9-dimethoxy-1,2,3,4,4a,10b-hexahydro-phenanthridin-6-yl)-N-(3-morpholin-4-yl-propyl)-benzamide,
4-((2RS,4aRS,10bRS)-2-Hydroxy-8,9-dimethoxy-1,2,3,4,4a,10b-hexahydro-phenanthridin-6-yl)-N-(4-methyl-piperazin-1-yl)-benzamide,
4-((2RS,4aRS,10bRS)-2-Hydroxy-8,9-dimethoxy-1,2,3,4,4a,10b-hexahydro-phenanthridin-6-yl)-N-morpholin-4-yl-benzamide,
({1-[4-((2RS,4aRS,10bRS)-2-Hydroxy-8,9-dimethoxy-1,2,3,4,4a,10b-hexahydro-phenanthridin-6-yl)-phenyl]-methanoyl}-methyl-amino)-acetic acid methyl ester,
4-((2RS,4aRS,10bRS)-2-Hydroxy-8,9-dimethoxy-1,2,3,4,4a,10b-hexahydro-phenanthridin-6-yl)-N-quinolin-3-yl-benzamide,
4-((2RS,4aRS,10bRS)-2-Hydroxy-8,9-dimethoxy-1,2,3,4,4a,10b-hexahydro-phenanthridin-6-yl)-N-(2-pyridin-2-yl-ethyl)-benzamide,
1-[4-((2RS,4aRS,10bRS)-2-Hydroxy-8,9-dimethoxy-1,2,3,4,4a,10b-hexahydro-phenanthridin-6-yl)-phenyl]-1-(4-pyridin-2-yl-piperazin-1-yl)-methanone,
1-[4-((2RS,4aRS,10bRS)-2-Hydroxy-8,9-dimethoxy-1,2,3,4,4a,10b-hexahydro-phenanthridin-6-yl)-phenyl]-1-[4-(2-morpholin-4-yl-ethyl)-piperazin-1-yl]-methanone,
N-Ethyl-4-((2RS,4aRS,10bRS)-2-hydroxy-8,9-dimethoxy-1,2,3,4,4a,10b-hexahydro-phenanthridin-6-yl)-N-(2-methoxyethyl)-benzamide,
N-Cyclopropyl-4-((2RS,4aRS,10bRS)-2-hydroxy-8,9-dimethoxy-1,2,3,4,4a,10b-hexahydro-phenanthridin-6-yl)-benzamide,
2-(4-{1-[4-((2RS,4aRS,10bRS)-2-Hydroxy-8,9-dimethoxy-1,2,3,4,4a,10b-hexahydro-phenanthridin-6-yl)-phenyl]-methanoyl}-piperazin-1-yl)-1-pyrrolidin-1-yl-ethanone,
2-(4-{1-[4-((2RS,4aRS,10bRS)-2-Hydroxy-8,9-dimethoxy-1,2,3,4,4a,10b-hexahydro-phenanthridin-6-yl)-phenyl]-methanoyl}-piperazin-1-yl)-N-pyridin-3-yl-acetamide,
4-((2RS,4aRS,10bRS)-2-Hydroxy-8,9-dimethoxy-1,2,3,4,4a,10b-hexahydro-phenanthridin-6-yl)-N,N-dimethyl-benzamide,
2-(4-{1-[4-((2RS,4aRS,10bRS)-2-Hydroxy-8,9-dimethoxy-1,2,3,4,4a,10b-hexahydro-phenanthridin-6-yl)-phenyl]-methanoyl}-piperazin-1-yl)-N-pyridin-2-yl-acetamide,
2-(4-{1-[4-((2R,4aR,10bR)-2-Hydroxy-8,9-dimethoxy-1,2,3,4,4a,10b-hexahydro-phenanthridin-6-yl)-phenyl]-methanoyl}-piperazin-1-yl)-N,N-dimethyl-acetamide,
2-(4-{1-[4-((2RS,4aRS,10bRS)-2-Hydroxy-8,9-dimethoxy-1,2,3,4,4a,10b-hexahydro-phenanthridin-6-yl)-phenyl]-methanoyl}-piperazin-1-yl)-1-morpholin-4-yl-ethanone,
1-[4-((2RS,4aRS,10bRS)-2-Hydroxy-8,9-dimethoxy-1,2,3,4,4a,10b-hexahydro-phenanthridin-6-yl)-phenyl]-1-(4-pyridin-4-yl-piperazin-1-yl)-methanone,
1-[4-((2RS,4aRS,10bRS)-2-Hydroxy-8,9-dimethoxy-1,2,3,4,4a,10b-hexahydro-phenanthridin-6-yl)-phenyl]-1-morpholin-4-yl-methanone,
4-((2RS,4aRS,10bRS)-2-Hydroxy-8,9-dimethoxy-1,2,3,4,4a,10b-hexahydro-phenanthridin-6-yl)-N-(2-pyridin-4-yl-ethyl)-benzamide,
4-((2RS,4aRS,10bRS)-2-Hydroxy-8,9-dimethoxy-1,2,3,4,4a,10b-hexahydro-phenanthridin-6-yl)-N-(2-pyridin-3-yl-ethyl)-benzamide,
4-((2RS,4aRS,10bRS)-2-Hydroxy-8,9-dimethoxy-1,2,3,4,4a,10b-hexahydro-phenanthridin-6-yl)-benzoic acid N′-(1-morpholin-4-yl-methanoyl)-hydrazide,
N-(2,6-Dimethoxy-pyridin-3-yl)-4-((2RS,4aRS,10bRS)-2-hydroxy-8,9-dimethoxy-1,2,3,4,4a,10b-hexahydro-phenanthridin-6-yl)-benzamide,
4-[(2RS,4aRS,10bRS)-9-(1,1-Difluoro-methoxy)-2-hydroxy-8-methoxy-1,2,3,4,4a,10b-hexahydro-phenanthridin-6-yl]-N,N-dimethyl-benzamide,
N-Cyclopropyl-4-[(2RS,4aRS,10bRS)-9-(1,1-difluoro-methoxy)-2-hydroxy-8-methoxy-1,2,3,4,4a,10b-hexahydro-phenanthridin-6-yl]-benzamide,
4-[(2RS,4aRS,10bRS)-9-(1,1-Difluoro-methoxy)-2-hydroxy-8-methoxy-1,2,3,4,4a,10b-hexahydro-phenanthridin-6-yl]-N,N-bis-(2-methoxy-ethyl)-benzamide,
4-[(2RS,4aRS,10bRS)-9-(1,1-Difluoro-methoxy)-2-hydroxy-8-methoxy-1,2,3,4,4a,10b-hexahydro-phenanthridin-6-yl]-N-(2-morpholin-4-yl-ethyl)-benzamide,
4-[(2RS,4aRS,10bRS)-9-(1,1-Difluoro-methoxy)-2-hydroxy-8-methoxy-1,2,3,4,4a,10b-hexahydro-phenanthridin-6-yl]-N-(3-morpholin-4-yl-propyl)-benzamide,
1-{4-[(2RS,4aRS,10bRS)-9-(1,1-Difluoro-methoxy)-2-hydroxy-8-methoxy-1,2,3,4,4a,10b-hexahydro-phenanthridin-6-yl]-phenyl}-1-[4-(2-morpholin-4-yl-ethyl)-piperazin-1-yl]-methanone,
1-{4-[(2RS,4aRS,10bRS)-9-(1,1-Difluoro-methoxy)-2-hydroxy-8-methoxy-1,2,3,4,4a,10b-hexahydro-phenanthridin-6-yl]-phenyl}-1-(4-pyridin-4-yl-piperazin-1-yl)-methanone,
2-[4-(1-{4-[(2RS,4aRS,10bRS)-9-(1,1-Difluoro-methoxy)-2-hydroxy-8-methoxy-1,2,3,4,4a,10b-hexahydro-phenanthridin-6-yl]-phenyl}-methanoyl)-piperazin-1-yl]-N-pyridin-2-yl-acetamide,
2-[4-(1-{4-[(2RS,4aRS,10bRS)-9-(1,1-Difluoro-methoxy)-2-hydroxy-8-methoxy-1,2,3,4,4a,10b-hexahydro-phenanthridin-6-yl]-phenyl}-methanoyl)-piperazin-1-yl]-1-morpholin-4-yl-ethanone,
1-{4-[(2RS,4aRS,10bRS)-9-(1,1-Difluoro-methoxy)-2-hydroxy-8-methoxy-1,2,3,4,4a,10b-hexahydro-phenanthridin-6-yl]-phenyl}-1-pyrrolidin-1-yl-methanone,
2-[4-(1-{4-[(2RS,4aRS,10bRS)-9-(1,1-Difluoro-methoxy)-2-hydroxy-8-methoxy-1,2,3,4,4a,10b-hexahydro-phenanthridin-6-yl]-phenyl}-methanoyl)-piperazin-1-yl]-N,N-dimethyl-acetamide,
1-{4-[(2RS,4aRS,10bRS)-9-(1,1-Difluoro-methoxy)-2-hydroxy-8-methoxy-1,2,3,4,4a,10b-hexahydro-phenanthridin-6-yl]-phenyl}-1-[4-(2-dimethylamino-ethyl)-piperazin-1-yl]-methanone,
N-(2,6-Dimethoxy-pyridin-3-yl)-4-((2R,4aR,10bR)-2-hydroxy-8,9-dimethoxy-1,2,3,4,4a,10b-hexahydro-phenanthridin-6-yl)-benzamide,
N-(2,6-Dimethoxy-pyridin-3-yl)-4-((2S,4aS,10bS)-2-hydroxy-8,9-dimethoxy-1,2,3,4,4a,10b-hexahydro-phenanthridin-6-yl)-benzamide,
N-Cyclopropyl-4-[(2R,4aR,10bR)-9-(1,1-difluoro-methoxy)-2-hydroxy-8-methoxy-1,2,3,4,4a,10b-hexahydro-phenanthridin-6-yl]-benzamide,
N-Cyclopropyl-4-[(2S,4aS,10bS)-9-(1,1-difluoro-methoxy)-2-hydroxy-8-methoxy-1,2,3,4,4a,10b-hexahydro-phenanthridin-6-yl]-benzamide,
N-Cyclopropyl-4-((2R,4aR,10bR)-9-ethoxy-2-hydroxy-8-methoxy-1,2,3,4,4a,10b-hexahydro-phenanthridin-6-yl)-benzamide,
N-Cyclobutyl-4-((2R,4aR,10bR)-9-ethoxy-2-hydroxy-8-methoxy-1,2,3,4,4a,10b-hexahydro-phenanthridin-6-yl)-benzamide,
4-((2R,4aR,10bR)-9-Ethoxy-2-hydroxy-8-methoxy-1,2,3,4,4a,10b-hexahydro-phenanthridin-6-yl)-N,N-diisopropyl-benzamide,
N-Cyclopropyl-3-((2R,4aR,10bR)-9-ethoxy-2-hydroxy-8-methoxy-1,2,3,4,4a,10b-hexahydro-phenanthridin-6-yl)-benzamide,
N-Cyclobutyl-3-((2R,4aR,10bR)-9-ethoxy-2-hydroxy-8-methoxy-1,2,3,4,4a,10b-hexahydro-phenanthridin-6-yl)-benzamide,
3-((2R,4aR,10bR)-9-Ethoxy-2-hydroxy-8-methoxy-1,2,3,4,4a,10b-hexahydro-phenanthridin-6-yl)-N,N-diisopropyl-benzamide,
N-Cyclopropyl-4-((3S,4aR,10bR)-9-ethoxy-3-hydroxy-8-methoxy-1,2,3,4,4a,10b-hexahydro-phenanthridin-6-yl)-benzamide,
and the enantiomers, salts, N-oxides, salts of the N-oxides and enantiomers thereof.

11. A compound of formula I according to claim 1, which have with respect to the positions 4a and 10b the configuration shown in formula I*: 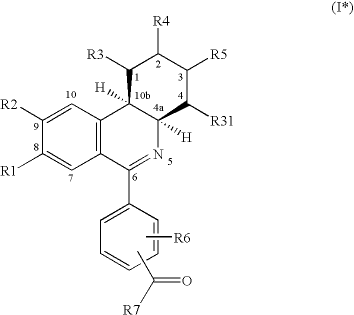
or a salt, N-oxide or salt of an N-oxide thereof.

12. A compound of formula I according to claim 1, which have with respect to the positions 2, 4a and 10b the configuration shown in formula Ia*****, or, which have with respect to the positions 3, 4a and 10b the configuration shown in formula Ib*****: 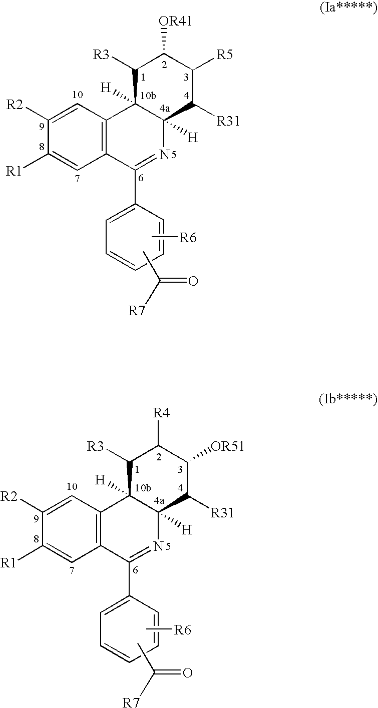
or a salt, N-oxide or salt of an N-oxide thereof.

13. (canceled)

14. A pharmaceutical composition comprising one or more compounds of formula I as claimed in claim 1, or a pharmaceutically acceptable salt, enantiomer, N-oxide, salt of an N-oxide or enantiomer thereof, together with a pharmaceutically acceptable excipient and/or vehicle.

15.-16. (canceled)

17. A method for treating an illness in a patient comprising administering to said patient a therapeutically effective amount of a compound of formula I as claimed in claim 1, or a pharmaceutically acceptable salt, enantiomer, N-oxide, salt of an N-oxide or enantiomer thereof.

18. A method for treating an airway disorder in a patient comprising administering to said patient a therapeutically effective amount of a compound of formula I as claimed in claim 1, or a pharmaceutically acceptable salt, enantiomer, N-oxide, salt of an N-oxide or enantiomer thereof.

19. A method for treating a PDE-mediated disorder in a patient comprising administering to said patient a therapeutically effective amount of a compound of formula I as claimed in claim 1, or a pharmaceutically acceptable salt, enantiomer, N-oxide, salt of an N-oxide or enantiomer thereof.
